(19) United States
(12) Patent Application Publication
(10) Pub. No.: US 2026/0034156 A1
(43) Pub. Date: Feb. 5, 2026

(54) INHIBITORS OF SARM1 IN COMBINATION WITH NAD+ OR A NAD+ PRECURSOR

(71) Applicant: Disarm Therapeutics, Inc., Indianapolis, IN (US)

(72) Inventors: Todd BOSANAC, New Milford, CT (US); Rajesh DEVRAJ, Chesterfield, MO (US); Thomas ENGBER, Boston, MA (US); Robert Owen HUGHES, Newtown, CT (US); Raul Eduardo KRAUSS, Chestnut Hill, MA (US)

(21) Appl. No.: 19/358,248

(22) Filed: Oct. 14, 2025

Related U.S. Application Data
(63) Continuation of application No. 17/278,716.
(60) Provisional application No. 62/748,000, filed on Oct. 19, 2018.

(51) Int. Cl. A61K 31/706; A61P 25/28; C12N 15/113; C12N 15/115

(52) U.S. Cl. CPC A61K 31/706; A61P 25/28; C12N 15/113; C12N 15/115

(57) ABSTRACT

The present disclosure relates to methods of treating neurodegeneration and neurodegenerative diseases comprising administering to a subject in need thereof a combination of a SARM1 inhibitor and NAD+ or a NAD+ precursor.

### CROSS REFERENCE TO RELATED APPLICATIONS

This application is a Continuation of U.S. patent application Ser. No. 17/278,716 filed on Mar. 23, 2021 which is a 35 U.S.C. § 371 National Stage application with International Application Number PCT/US2019/056914 and international filing date of Oct. 18, 2019 which claims the benefit of Provisional Patent Application No. 62/748,000 filed Oct. 19, 2018, which is herein incorporated by reference in its entirety.

### BACKGROUND

Axonal degeneration is a hallmark of several neurological disorders including peripheral neuropathy, traumatic brain injury, and neurodegenerative diseases (Gerdts et al., SARM1 activation triggers axon degeneration locally via nicotinamide adenine dinucleotide (NAD+) destruction. Science 348 2015, pp. 453-457, hereby incorporated by reference in its entirety). Neurodegenerative diseases and injuries are devastating to both patients and caregivers. Costs associated with these diseases currently exceed several hundred billion dollars annually in the Unites States alone. Since the incidence of many of these diseases and disorders increases with age, their incidence is rapidly increasing as demographics change.

### SUMMARY

Axonal degeneration after an injury is characterized by the sequential depletion of nicotinamide mononucleotide adenylyltransferase (NMNAT), NAD+ and adenosine tri-phosphate (ATP), followed by neurofilament proteolysis and axonal fragmentation occurring approximately 8 to 24 hours after the original injury (Gerdts, J., et al., Neuron, 2016, 89, 449-460, hereby incorporated by reference in its entirety). Following axonal damage, Sterile Alpha and TIR motif-containing 1 (SARM1) serves as the central executioner in the axonal degeneration pathway. Activated SARM1 is a highly effective NADase that depletes local axonal NAD+ reserves within minutes to a few hours after activation, leading to a local bioenergetic crisis, followed by rapid axonal degeneration. The present disclosure shows the surprising discovery that the combination of nicotinamide adenine dinucleotide (NAD+) or a NAD+ precursor (e.g., NR, NA, NaR, NAM, NMN, NaMN, TRP, vitamin B₃, or NAAD) and a SARM1 inhibitor provides vastly superior and longer lasting axonal protection over the effect of either agent alone. In some embodiments, such combination provides a safe and effective approach to treat patients with axonopathies.

Accordingly, in some embodiments, the present disclosure encompasses the recognition that a combination of NAD+ or a NAD+ precursor (e.g., NR, NA, NaR, NAM, NMN, NaMN, TRP, vitamin B₃, or NAAD) and a SARM1 inhibitor maintains higher intracellular NAD+ levels, thereby preventing, ameliorating and/or decreasing the progression of axonal degeneration and cell death. In some embodiments, such combination substantially delays the pathological SARM1-mediated decrease in intracellular NAD+ that occurs as a result of SARM1 activation.

In some embodiments, the present disclosure provides a method for treating, preventing, and/or ameliorating a neurodegenerative disease, disorder or condition comprising administering a SARM1 inhibitor in combination with NAD+ or a NAD+ precursor (e.g., NR, NA, NaR, NAM, NMN, NaMN, TRP, vitamin B₃, or NAAD).

In some embodiments, a neurodegenerative disease, disorder, or condition is associated with axonal degeneration (e.g., axonal fragmentation or degradation). Accordingly, in some embodiments, the present disclosure provides a method of treating, preventing, and/or ameliorating axonal degeneration comprising administering to a subject in need thereof a SARM1 inhibitor in combination with NAD+ or a NAD+ precursor (e.g., NR, NA, NaR, NAM, NMN, NaMN, TRP, vitamin B₃, or NAAD). In some embodiments, the axonal degeneration results from reduction or depletion of NAD+.

In some embodiments, provided methods prevent or slow the progression of degeneration of the axon distal to an axonal injury. In some embodiments, provided methods treat or prevent secondary conditions associated with neurodegenerative disorders. Such secondary conditions include, but are not limited to, muscle impairments, respiratory impairments, anxiety, depression, speech impairments, pulmonary embolisms, cardiac arrhythmias, and/or pneumonia.

In some embodiments, the present disclosure relates to a method of treating, preventing, and/or ameliorating a neurodegenerative disease, disorder or condition comprising i) providing a) a subject diagnosed with, at risk for, or exhibiting symptoms of, a neurodegenerative disease, disorder, or condition and b) a combination comprising a SARM1 inhibitor and NAD+ or a NAD+ precursor (e.g., NR, NA, NaR, NAM, NMN, NaMN, TRP, vitamin B₃, or NAAD); and ii) administering said combination to said subject under conditions such that said neurodegenerative disease, disorder, or condition is reduced.

In some embodiments, the present disclosure relates to a method of treating, preventing, and/or ameliorating a neurodegenerative disease, disorder or condition comprising i) providing a) a subject diagnosed with, at risk for, or exhibiting symptoms of, a neurodegenerative disease, disorder, or condition and b) a SARM1 inhibitor; and ii) administering the SARM1 inhibitor to a subject who is or has been exposed to NAD+ or a NAD+ precursor (e.g., NR, NA, NaR, NAM, NMN, NaMN, TRP, vitamin B₃, or NAAD) under conditions such that said neurodegenerative disease, disorder, or condition is reduced.

In some embodiments, the present disclosure provides a combination therapy comprising a SARM1 inhibitor and NAD+ or a NAD+ precursor (e.g., NR, NA, NaR, NAM, NMN, NaMN, TRP, vitamin B₃, or NAAD). In some embodiments, provided combination therapies comprise a SARM1 inhibitor, NAD+ or a NAD+ precursor (e.g., NR, NA, NaR, NAM, NMN, NaMN, TRP, vitamin B₃, or NAAD), and one or more additional therapeutic agents.

In some embodiments, provided combination therapies are useful for treating, preventing, and/or ameliorating neurodegenerative diseases, disorders or conditions. In some embodiments, provided combination therapies are useful for treating, preventing, and/or ameliorating axonal degeneration. In some embodiments, provided combination therapies are useful for preventing or slowing the progression of degeneration of the axon distal to an axonal injury. In some embodiments, provided combination therapies are useful for maintaining the function of an axon including, but not limited to, metabolism, axonal integrity, intracellular transport, and action potential propagation.

In some embodiments, a neurodegenerative disease, disorder or condition is characterized by axons that are susceptible to disruption, degeneration or pathological stress. In some embodiments, such diseases, disorders or conditions include, but are not limited to, cancer, diabetes, neurodegenerative diseases, cardiovascular disease, blood clotting, inflammation, flushing, obesity, aging, or stress.

In some embodiments, a neurodegenerative disease, disorder or condition is selected from the group consisting neuropathies or axonopathies. In some embodiments, a neuropathy or axonopathy is associated with axonal degeneration.

In some embodiments, a neuropathy associated with axonal degeneration is a hereditary or congenital neuropathy or axonopathy. In some embodiments, a neuropathy associated with axonal degeneration results from a de novo or somatic mutation. In some embodiments, a neuropathy associated with axonal degeneration results from idiopathic conditions.

In some embodiments, a neuropathy or axonopathy associated with axonal degeneration, includes, but is not limited to, Parkinson's disease, Alzheimer's disease, Huntington's disease, Herpes infection, diabetes, amyotrophic lateral sclerosis (ALS), multiple sclerosis, a demyelinating disease, ischemia or stroke, frontotemporal dementia, ataxias, Charcot Marie Tooth, neuromyelitis optica, traumatic brain injury, chemical injury, thermal injury, and AIDS.

In some embodiments, a neurodegenerative disease, disorder or condition may be or comprises a traumatic neuronal injury. In some embodiments, a traumatic neuronal injury is a blunt-force trauma, a closed-head injury, an open-head injury, exposure to a concussive and/or explosive force, a penetrating injury in or to the brain cavity or innervated region of the body. In some embodiments, a traumatic neuronal injury is a force which causes axons to deform, stretch, crush or sheer.

In some embodiments, subjects to which a combination therapy as described herein is administered are suffering from or susceptible to a neurodegenerative disease, disorder or condition. In some embodiments, the subject is at risk of developing a neurodegenerative disease, disorder or condition. In some embodiments, the subject is elderly. In some embodiments, the subject has genetic risk factors for neurodegeneration.

In some embodiments, the subject is at risk of developing a disease, disorder, or condition characterized by axonal degeneration. In some embodiments, the subject has a disease, disorder, or condition characterized by axonal degeneration. In some embodiments, the subject has been diagnosed with a disease, disorder, or condition characterized by axonal degeneration. In some embodiments, the subject has not been diagnosed with a disease, disorder, or condition characterized by axonal degeneration.

In some embodiments, provided methods comprise administering a combination therapy as described herein to a subject population in need thereof. In some embodiments, the subject population is elderly. In some embodiments, the subject population has genetic risk factors for neurodegeneration.

In some embodiments, the subject population is drawn from individuals who engage in activities where the potential for traumatic neuronal injury is high. In some embodiments, the subject population is drawn from athletes who engage in contact sports or other high-risk activities.

In certain embodiments, a combination comprising a SARM1 inhibitor and NAD+ or a NAD+ precursor (e.g., NR, NA, NaR, NAM, NMN, NaMN, TRP, vitamin B₃, or NAAD) is useful, for example, as an analytical tool, as a probe in biological assays, or as a therapeutic agent in accordance with the present disclosure.

Such combinations provided by this disclosure are also useful for the study of SARM1 NADase function in biological and pathological phenomena and the comparative evaluation of new SARM1 activity inhibitors in vitro or in vivo. In some embodiments, a combination comprising a SARM1 inhibitor and NAD+ or a NAD+ precursor (e.g., NR, NA, NaR, NAM, NMN, NaMN, TRP, vitamin B₃, or NAAD) is useful for studying axonal integrity. In some embodiments, such combinations are useful for studying apoptosis.

In some embodiments, the present disclosure provides a method for inhibiting the degeneration of neurons derived from a subject comprising administering to the subject a SARM1 inhibitor in combination with NAD+ or a NAD+ precursor (e.g., NR, NA, NaR, NAM, NMN, NaMN, TRP, vitamin B₃, or NAAD).

In some embodiments, provided combinations are useful for inhibiting the degeneration of a neuron, or a portion thereof. In some embodiments, provided combinations are useful to treat neurons whose axons are injured. In some embodiments, provided combinations are useful for inhibiting the degeneration of a neuron, or a portion thereof, in vivo. In some embodiments, provided combinations are useful as stabilizing agents to promote in vitro neuronal survival.

In some embodiments, the present disclosure relates to a method of increasing intracellular concentrations of NAD+ comprising: contacting a cell with a SARM1 inhibitor and NAD+ or a NAD+ precursor (e.g., NR, NA, NaR, NAM, NMN, NaMN, TRP, vitamin B₃, or NA AD).

In some embodiments, provided SARM1 inhibitors reduce or inhibit binding of NAD+ by SARM1. In some embodiments, provided SARM1 inhibitors bind to SARM1 within a pocket comprising one or more catalytic residues (e.g., a catalytic cleft of SARM1). In some embodiments, provided SARM1 inhibitors bind to non-catalytic residues. In some such embodiments, provided SARM1 inhibitors are allosteric modulators of SARM1 activity. Accordingly, in some embodiments, the present disclosure provides a method of reducing or inhibiting binding of SARM1 by NAD+ comprising administering to a subject in need thereof a combination of a SARM1 inhibitor and NAD+ or a NAD+ precursor (e.g., NR, NA, NaR, NAM, NMN, NaMN, TRP, vitamin B₃, or NAAD). In some embodiments, such SARM1 inhibitor binds to one or more catalytic residues in the binding pocket of SARM1.

In some embodiments, SARM1 inhibitors and NAD+ or a NAD+ precursor (e.g., NR, NA, NaR, NAM, NMN, NaMN, TRP, vitamin B₃, or NAAD) are co-administered to a subject. In some embodiments, a subject is first administered a SARM1 inhibitor followed by administration of NAD+ or a NAD+ precursor (e.g., NR, NA, NaR, NAM, NMN, NaMN, TRP, vitamin B₃, or NAAD). In some embodiments, NAD+ or a NAD+ precursor (e.g., NR, NA, NaR, NAM, NMN, NaMN, TRP, vitamin B₃, or NAAD) is administered prior to the SARM1 inhibitor. In some embodiments, a SARM1 inhibitor is administered to a subject exposed to NAD+ or a NAD+ precursor (e.g., NR, NA, NaR, NAM, NMN, NaMN, TRP, vitamin B₃, or NAAD).

In some embodiments, provided methods and/or combination therapies inhibit activity of SARM1. Alternatively or additionally, in some embodiments, provided methods and/or combination therapies alleviate one or more attributes of neurodegeneration. In some embodiments, the present disclosure provides methods of treating, preventing, and/or ameliorating a neurodegenerative disease, disorder or condition associated with axonal degeneration.

In some embodiments, the SARM1 inhibitor is a small molecule, a polypeptide, a peptide fragment, a nucleic acid (e.g., a siRNA, an antisense oligonucleotide, a micro-RNA, or an aptamer), an antibody, or a ribozyme.

In some embodiments, the SARM1 inhibitor is a small molecule. In some embodiments, the SARM1 inhibitor is a siRNA. In some embodiments, the SARM1 inhibitor is an antisense oligonucleotide. In some embodiments, the SARM1 inhibitor is a polypeptide. In some embodiments, a SARM1 inhibitor is a peptide fragment. In some embodiments, a SARM1 inhibitor is a nucleic acid. In some embodiments, a SARM1 inhibitor is an antisense oligonucleotide.

In some embodiments, the present disclosure provides compositions that comprise and/or deliver a SARM1 inhibitor (e.g., in a form as described herein), a prodrug or active metabolite thereof. In certain embodiments, a composition comprising a SARM1 inhibitor is formulated for use in administering to a subject in combination with NAD+ or a NAD+ precursor (e.g., NR, NA, NaR, NAM, NMN, NaMN, TRP, vitamin B₃, or NAAD).

In some embodiments, the present disclosure provides pharmaceutical compositions and nutritional supplements containing NAD+ or a NAD+ precursor (e.g., NR, NA, NaR, NAM, NMN, NaMN, TRP, vitamin B₃, or NAAD). In some embodiments, the present disclosure relates to methods of using NAD+ or a NAD+ precursor (e.g., NR, NA, NaR, NAM, NMN, NaMN, TRP, vitamin B₃, or NAAD) that promote the increase of intracellular levels of nicotinamide adenine dinucleotide (NAD+) in cells and tissues for improving cell and tissue survival. In some embodiments, the present disclosure provides a medium containing NAD+ or a NAD+ precursor (e.g., NR, NA, NaR, NAM, NMN, NaMN, TRP, vitamin B₃, or NAAD) disclosed herein. In further embodiments, the present disclosure relates to methods of using NAD+ or a NAD+ precursor (e.g., NR, NA, NaR, NAM, NMN, NaMN, TRP, vitamin B₃, or NAAD) for increasing NAD+ levels in cells and tissues and for improving cell and tissue survival.

In some embodiments, the present disclosure provides compositions comprising a SARM1 inhibitor for use in combination with NAD+ or a NAD+ precursor (e.g., NR, NA, NaR, NAM, NMN, NaMN, TRP, vitamin B₃, or NAAD). In some embodiments, such compositions are pharmaceutical compositions that include at least one pharmaceutically acceptable carrier, diluent or excipient.

In some embodiments, the SARM1 inhibitors can be identified according to, e.g., the assays described in WO 2018/057989, published on Mar. 29, 2018, which is hereby incorporated by reference in its entirety.

### BRIEF DESCRIPTION OF THE DRAWING

FIGS. 1A and 1B show that the combination of compound I-26+NR extends neuroprotection post-axotomy as compared to single agent therapy. For each concentration of compound I-26 tested, the extent of axonal protection of a combination of compound I-26+NR was compared to the amount of protection produced by the agent in that combination that, individually, had greater protective effect. FIGS. 1A and 1B show the degeneration index of DRG axons at 16 and 24 hours post-axotomy, respectively. The degeneration index of uncut axons, untreated cut axons, axons treated with 100 μM NR, 1.1 or 3.3 μM compound I-26 alone, and 1.1 or 3.3 μM compound I-26+100 μM NR are indicated. Statistical significance is indicated by * (p<0.05); ** (p<0.01); *** (p<0.001); and **** (p<0.0001). In FIG. 1A, at 16 h, I-26 or NR alone provided a modest amount of axonal protection, which is similar for both agents. The combination of compound I-26+NR provided a statistically significant and substantially greater protection than either compound I-26 or NR alone. In FIG. 1B, at 24 h, NR alone provided a modest level of protection, whereas 1.1 μM of compound I-26 alone afforded no statistically significant benefit. Surprisingly, the combination of 1.1 μM compound I-26+NR provided robust and statistically significant protection. Furthermore, the magnitude of the combined effect of compound I-26 and NR is greater than the sum of the individual effects of either agent alone, indicating that the effect of the combination is not simply additive but in fact synergistic and could not have been predicted from the individual effect of each agent in isolation. At the higher 3.3 μM dose of compound I-26 axons show more protection than NR alone and the combination of 3.3 μM compound I-26+NR showed a statistically significant benefit than compound I-26 alone.

FIGS. 2A and 2B show that the combination of compound I-86+NR extends neuroprotection post-axotomy as compared to single agent therapy. For each concentration of compound I-86 tested, the extent of axonal protection of a combination of compound I-86+NR was compared to the amount of protection produced by the agent in that combination that, individually, had greater protective effect. FIGS. 2A and 2B show the degeneration index of DRG axons at 16 and 24 hours post-axotomy, respectively. The degeneration index of uncut axons, untreated cut axons, axons treated with 100 μM NR, 1.1, 3.3 or 10 μM compound I-86 alone, and 1.1, 3.3 or 10 μM compound I-86+100 μM NRare indicated. Statistical significance is indicated by * (p<0.05); ** (p<0.01); *** (p<0.001); and **** (p<0.0001). In FIG. 2A, at 16 h, NR alone provided greater protection than 1.1 μM compound I-86 alone, whereas 3.3 μM compound I-86 alone provided greater protection than NR alone. The protection afforded by the combination of 1.1 μM compound I-86+NR was stronger than, and statistically different from, the protection observed with NR alone. The protection afforded by the combination of 3.3 μM compound I-86+NR was stronger than, and statistically different from, the protection observed with 3.3 μM compound I-86 alone. In FIG. 2B, at 24 h, 1.1 μM compound I-86 alone provided less protection than NR alone, whereas 3.3 μM compound I-86 alone and 10 μM compound I-86 offered similar protection to NR alone. The protection afforded by the combinations of compound I-86+NR (3.3 μM compound I-86+NR and 10 μM compound I-86+NR) was stronger than, and statistically significant from the protection observed with either compound I-86 or NR alone.

FIGS. 3A and 3B show that the combination of compound II-6+NR extends neuroprotection post-axotomy as compared to single agent therapy. For each concentration of compound II-6 tested, the extent of axonal protection of a combination of compound II-6+NR was always compared to the amount of protection produced by the agent in that combination that, individually, had greater protective effect. FIGS. 3A and 3B show the degeneration index of DRG axons at 16 and 24 hours post-axotomy, respectively. The degeneration index of uncut axons, untreated cut axons, axons treated with 100 μM NR, 1.1 or 3.3 μM compound II-6 alone, and 1.1 or 3.3 μM compound II-6+100 μM NRare indicated. Statistical significance is indicated by * (p<0.05); ** (p<0.01); *** (p<0.001); and **** (p<0.0001). In FIG. 3A, at 16 h, the protection afforded by the combination of 1.1 μM compound II-6+NR was stronger than, and statistically different from, the protection observed with either compound II-6 or NR alone. At 3.3 μM, compound II-6 alone showed stronger protection than NR alone; however, the protection afforded by the combination of 3.3 μM compound II-6+NR was stronger than, and statistically different from, the protection observed with 3.3 μM compound II-6 alone. In FIG. 3B, at 24 h, the protection afforded by the combination of 1.1 μM compound II-6+NR was stronger than, and statistically different from, the protection observed with either compound II-6 or NR alone. At 3.3 μM, compound II-6 alone showed stronger protection than NR alone; however, the protection afforded by the combination of 3.3 μM compound II-6+NR was stronger than, and statistically different from, the protection observed with 3.3 μM compound II-6 alone.

FIGS. 4A and 4B show that the combination of compound II-32+NR extends neuroprotection post-axotomy as compared to single agent therapy. For each concentration of compound II-32 tested, the extent of axonal protection of a combination of compound II-32+NR was always compared to the amount of protection produced by the agent in that combination that, individually, had greater protective effect. FIGS. 4A and 4B show the degeneration index of DRG axons at 16 and 24 hours post-axotomy, respectively. The degeneration index of uncut axons, untreated cut axons, axons treated with 100 μM NR, 0.11, 0.33 or 1 μM compound II-32 alone, and compound II-32+100 μM NR are indicated. Statistical significance is indicated by * (p<0.05); ** (p<0.01); *** (p<0.001); **** (p<0.0001). In FIG. 4A, at 16 h, NR alone provided greater protection than 0.11 μM and 0.33 μM of compound II-32 alone, whereas 1 μM compound II-32 alone provided greater protection than NR alone. The protection afforded by each combination of 0.11 μM compound II-32+NR and 0.33 μM compound II-32+NR was stronger than, and statistically different from, the protection observed with NR alone. Similarly, the protection afforded by the combination of 1 μM compound II-32+NR was stronger than, and statistically different from, the protection observed with 1 μM compound II-32 alone. In FIG. 4B, at 24 h, NR alone provided greater protection than 0.11 μM and 0.33 μM compound II-32 alone, whereas 1 μM compound II-32 alone provided greater protection than NR alone. The protection afforded by the combinations of 0.11 μM compound II-32+NR and 0.33 μM compound II-32+NR was stronger than, and statistically different from, the protection observed with NR alone. Similarly, the protection afforded by the combination of 1 μM compound II-32+NR was statistically better than the protection observed with 1 μM compound II-32 alone.

### DEFINITIONS

Binding: It will be understood that the term “binding”, as used herein, typically refers to an association (e.g., a non-covalent or covalent association) between or among two or more entities. “Direct” binding involves physical contact between entities or moieties; indirect binding involves physical interaction by way of physical contact with one or more intermediate entities. Binding between two or more entities can typically be assessed in any of a variety of contexts—including where interacting entities or moieties are studied in isolation or in the context of more complex systems (e.g., while covalently or otherwise associated with a carrier entity and/or in a biological system or cell).

Biological Sample: As used herein, the term “biological sample” typically refers to a sample obtained or derived from a biological source (e.g., a tissue or organism or cell culture) of interest, as described herein. In some embodiments, a source of interest comprises an organism, such as an animal or human. In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example, nucleic acids or proteins extracted from a sample or obtained by subjecting a primary sample to techniques such as amplification or reverse transcription of mRNA, isolation and/or purification of certain components, etc.

Biomarker: The term “biomarker” is used herein to refer to a to an entity, event, or characteristic whose presence, level, degree, type, and/or form, correlates with a particular biological event or state of interest, so that it is considered to be a “marker” of that event or state. To give but a few examples, in some embodiments, a biomarker may be or comprise a marker for a particular disease state, or for likelihood that a particular disease, disorder or condition may develop, occur, or reoccur. In some embodiments, a biomarker may be or comprise a marker for a particular disease or therapeutic outcome, or likelihood thereof. Thus, in some embodiments, a biomarker is predictive, in some embodiments, a biomarker is prognostic, in some embodiments, a biomarker is diagnostic, of the relevant biological event or state of interest. A biomarker may be or comprise an entity of any chemical class, and may be or comprise a combination of entities. For example, in some embodiments, a biomarker may be or comprise a nucleic acid, a polypeptide, a lipid, a carbohydrate, a small molecule, an inorganic agent (e.g., a metal or ion), or a combination thereof. In some embodiments, a biomarker is a cell surface marker. In some embodiments, a biomarker is intracellular. In some embodiments, a biomarker is detected outside of cells (e.g., is secreted or is otherwise generated or present outside of cells, e.g., in a body fluid such as blood, urine, tears, saliva, cerebrospinal fluid, etc. In some embodiments, a biomarker may be or comprise a genetic or epigenetic signature. In some embodiments, a biomarker may be or comprise a gene expression signature.

In some embodiments, a biomarker may be or comprise a marker for neurodegeneration, or for likelihood that a neurodegenerative disease, disorder or condition may develop, occur, or reoccur. In some embodiments, a biomarker may be or comprise a marker of neurodegeneration a therapeutic outcome, or likelihood thereof. Thus, in some embodiments, a biomarker is predictive, in some embodiments, a biomarker is prognostic, and in some embodiments, a biomarker is diagnostic, of a neurodegenerative disease, disorder or condition. In some embodiments changes in biomarker levels can be detected via cerebral spinal fluid (CSF), plasma and/or serum. In some embodiments a biomarker can be a detectable signal produced by medical imaging techniques including, but not limited to, magnetic resonance imaging (MRI), positron emission-tomography (PET), and/or computed tomography (CT). In some embodiments, a biomarker can be a detectable change in electrophysiological properties.

In some embodiments, neurodegeneration may be assessed, for example, by detecting an increase and/or decrease in the concentration of neurofilament light chain protein (NF-L) and/or neurofilament heavy chain protein (NF-H) contained in bodily fluids from a subject including, but not limited to, cerebral spinal fluid, blood, serum and/or plasma. In some embodiments, the incidence and/or progression of neurodegeneration can be assessed via positron emission tomography (PET) with a synaptic vesicle glycoprotein 2a (SV2A) ligand. In some embodiments, a detectable change in constitutive NAD+ and/or cADPR levels in neurons can be used to assess neurodegeneration.

In some embodiments, a detectable change in one or more neurodegeneration associated proteins in a subject, relative to a healthy reference population can be used as a biomarker of neurodegeneration. Such proteins include, but are not limited to, albumin, amyloid-β (Aβ)38, Aβ40, Aβ42, glial fibrillary acid protein (GFAP), heart-type fatty acid binding protein (hFABP), monocyte chemoattractin protein (MCP)-1, neurogranin, neuron specific enolayse (NSE), soluble amyloid precursor protein (sAPP)α, sAPPP, soluble triggering receptor expressed on myeloid cells (sTREM) 2, phospho-tau, and/or total-tau. In some embodiments, an increase in cytokines and/or chemokines, including, but not limited to, Ccl2, Ccl7, Ccl12, Csf1, and/or 116, can be used as a biomarker of neurodegeneration.

Carrier: As used herein, the term “carrier” refers to a diluent, adjuvant, excipient, or vehicle with which a composition is administered. In some exemplary embodiments, carriers can include sterile liquids, such as, for example, water and oils, including oils of petroleum, animal, vegetable or synthetic origin, such as, for example, peanut oil, soybean oil, mineral oil, sesame oil and the like. In some embodiments, carriers are or include one or more solid components.

Combination: The terms “combination therapy” or “in combination with”, as used herein, refer to those situations in which two or more different pharmaceutical agents for the treatment of disease are administered in overlapping regimens so that the subject is simultaneously exposed to at least two agents. In some embodiments, the different agents are administered simultaneously. In some embodiments, the administration of one agent overlaps the administration of at least one other agent. In some embodiments, the different agents are administered sequentially (e.g., all “doses” of a first regimen are administered prior to administration of any doses of a second regimen) such that the agents have simultaneous biologically activity within a subject. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).

Composition: Those skilled in the art will appreciate that the term “composition” may be used to refer to a discrete physical entity that comprises one or more specified components. In general, unless otherwise specified, a composition may be of any form—e.g., gas, gel, liquid, solid, etc.

Domain: The term “domain” as used herein refers to a section or portion of an entity. In some embodiments, a “domain” is associated with a particular structural and/or functional feature of the entity so that, when the domain is physically separated from the rest of its parent entity, it substantially or entirely retains the particular structural and/or functional feature. Alternatively or additionally, a domain may be or include a portion of an entity that, when separated from that (parent) entity and linked with a different (recipient) entity, substantially retains and/or imparts on the recipient entity one or more structural and/or functional features that characterized it in the parent entity. In some embodiments, a domain is a section or portion of a molecule (e.g., a small molecule, carbohydrate, lipid, nucleic acid, or polypeptide). In some embodiments, a domain is a section of a polypeptide; in some such embodiments, a domain is characterized by a particular structural element (e.g., a particular amino acid sequence or sequence motif, α-helix character, β-sheet character, coiled-coil character, random coil character, etc.), and/or by a particular functional feature (e.g., binding activity, enzymatic activity, folding activity, signaling activity, etc.).

Dosage form or unit dosage form: Those skilled in the art will appreciate that the term “dosage form” may be used to refer to a physically discrete unit of an active agent (e.g., a therapeutic or diagnostic agent) for administration to a subject. Typically, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or agent administered to a particular subject is determined by one or more attending physicians and may involve administration of multiple dosage forms.

Dosing regimen or therapeutic regimen: Those skilled in the art will appreciate that the terms “dosing regimen” and “therapeutic regimen” may be used to refer to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Excipient: as used herein, refers to a non-therapeutic agent that may be included in a pharmaceutical composition, for example, to provide or contribute to a desired consistency or stabilizing effect. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Inhibitory agent: As used herein, the term “inhibitory agent” refers to an entity, condition, or event whose presence, level, or degree correlates with decreased level or activity of a target. In some embodiments, an inhibitory agent may act directly (in which case it exerts its influence directly upon its target, for example, by binding to the target); in some embodiments, an inhibitory agent may act indirectly (in which case it exerts its influence by interacting with and/or otherwise altering a regulator of the target, so that level and/or activity of the target is reduced). In some embodiments, an inhibitory agent is one whose presence or level correlates with a target level or activity that is reduced relative to a particular reference level or activity (e.g., that observed under appropriate reference conditions, such as presence of a known inhibitory agent, or absence of the inhibitory agent in question, etc.).

Neurodegeneration: As used herein, the term “neurodegeneration” refers to a reduction in one or more features, structures, or characteristics of a neuron or neuronal tissue. In some embodiments, neurodegeneration is observed as a pathological reduction in an organism. Those skilled in the art will appreciate that neurodegeneration is associated with certain diseases, disorders and conditions, including those that affect humans. In some embodiments, neurodegeneration may be transient (e.g., as sometimes occurs in association with certain infections and/or chemical or mechanical disruptions); in some embodiments, neurodegeneration may be chronic and/or progressive (e.g., as is often associated with certain diseases, disorders or conditions such as, but not limited to, Parkinson's disease, amyotrophic lateral sclerosis, multiple sclerosis, Huntington's disease, or Alzheimer's disease). In some embodiments, neurodegeneration may be assessed, for example, by detecting in a subject an increase in a biomarker associated with neurodegeneration. In some embodiments, neurodegeneration may be assessed, for example, by detecting in a subject a decrease in a biomarker associated with neurodegeneration. Alternatively or additionally, in some embodiments, neurodegeneration may be assessed by magnetic resonance imaging (MRI), biomarkers containing cerebral spinal fluid, or other biomarkers observed in subjects. In some embodiments, neurodegeneration is defined as a score below 24 on the mini-mental state examination. In some embodiments, neurodegeneration refers to loss of synapses. In some embodiments, neurodegeneration refers to a reduction in neural tissue relating to a traumatic injury (e.g. exposure to an external force which disrupts the integrity of the neural tissue). In some embodiments, neurodegeneration refers to a reduction in peripheral neural tissue. In some embodiments, neurodegeneration refers to a reduction in central nervous tissue.

Nicotinamide adenine dinucleotide (NAD+) precursor: As used herein, the terms “nicotinamide adenine dinucleotide (NAD+) precursor” and “NAD+ precursor” refer to a compound that may participate in the NAD+ metabolic pathway. In some embodiments, a NAD+ precursor is vitamin B₃. In some embodiments, a NAD+ precursor is a form of vitamin B₃. In some embodiments, a NAD+ precursor is nicotinamide riboside (NR), also known as 1-(β-D-ribofuranosyl)nicotinamide or N-ribosylnicotinamide. In some embodiments, a NAD+ precursor is nicotinic acid (NA), also known as niacin. In some embodiments, a NAD+ precursor is nicotinic acid riboside (NaR). In some embodiments a NAD+ precursor is nicotinamide (NAM), also known as 3-pyridinecarboxamide, niacinamide, nicotinic acid amide, or nicotinic amide. In some embodiments, a NAD+ precursor is nicotinamide mononucleotide (NMN), also known as, nicotinamide ribonucleoside 5′-phosphate, nicotinamide D-ribonucleotide, β-nicotinamide ribose monophosphate, or nicotinamide nucleotide. In some embodiments, a NAD+ precursor is nicotinic acid mononucleotide (NaMN). In some embodiments, a NAD+ precursor is tryptophan (TRP), also known as (2S)-2-amino-3-(1H-indol-3-yl)propanoic acid or 2-amino-3-(1H-indol-3-yl)propanoic acid. In some embodiments, a NAD+ precursor is deamido-NAD+, also known as deamido-NAD, deamino-NAD+, or nicotinic acid adenine dinucleotide (NAAD). In some embodiments, a NAD+ precursor is nicotinic acid riboside, O-ethylnicotinate riboside, or O-methylnicotinate riboside (Yang et al., J. Med. Chem., 2007, 50 (26), 6458-6461). In some embodiments, a NAD+ precursor is β-nicotinamide riboside. In some embodiments, a NAD+ precursor is a nicotinate ester nucleoside derivative. In some embodiments, a NAD+ precursor is a triacetyl-O-ethylnicotinate riboside (Yang et al., J. Med. Chem., 2007, 50 (26), 6458-6461). In some embodiments, when contacted to a mammalian cell, a NAD+ precursor can stimulate an increase in NAD+ concentration.

Oral: The phrases “oral administration” and “administered orally” as used herein have their art-understood meaning referring to administration by mouth of a compound or composition.

Parenteral: The phrases “parenteral administration” and “administered parenterally” as used herein have their art-understood meaning referring to modes of administration other than enteral and topical administration, usually by injection, and include, without limitation, intravenous, intramuscular, intra-arterial, intrathecal, intracapsular, intraorbital, intracardiac, intradermal, intraperitoneal, transtracheal, subcutaneous, subcuticular, intraarticulare, subcapsular, subarachnoid, intraspinal, and intrasternal injection and infusion.

Patient: As used herein, the term “patient” refers to any organism to which a provided composition is or may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. In some embodiments, a patient is suffering from or susceptible to one or more disorders or conditions. In some embodiments, a patient displays one or more symptoms of a disorder or condition. In some embodiments, a patient has been diagnosed with one or more disorders or conditions. In some embodiments, the patient is receiving or has received certain therapy to diagnose and/or to treat a disease, disorder, or condition.

Pharmaceutical composition: As used herein, the term “pharmaceutical composition” refers to an active agent, formulated together with one or more pharmaceutically acceptable carriers. In some embodiments, the active agent is present in unit dose amount appropriate for administration in a therapeutic or dosing regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutically acceptable salt: The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). In some embodiments, pharmaceutically acceptable salts include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

Prevent or prevention: As used herein, the terms “prevent” or “prevention”, when used in connection with the occurrence of a disease, disorder, and/or condition, refer to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time.

Specific: The term “specific”, when used herein with reference to an agent having an activity, is understood by those skilled in the art to mean that the agent discriminates between potential target entities or states. For example, in some embodiments, an agent is said to bind “specifically” to its target if it binds preferentially with that target in the presence of one or more competing alternative targets. In many embodiments, specific interaction is dependent upon the presence of a particular structural feature of the target entity (e.g., an epitope, a cleft, a binding site). It is to be understood that specificity need not be absolute. In some embodiments, specificity may be evaluated relative to that of the binding agent for one or more other potential target entities (e.g., competitors). In some embodiments, specificity is evaluated relative to that of a reference specific binding agent. In some embodiments, specificity is evaluated relative to that of a reference non-specific binding agent. In some embodiments, the agent or entity does not detectably bind to the competing alternative target under conditions of binding to its target entity. In some embodiments, a binding agent binds with higher on-rate, lower off-rate, increased affinity, decreased dissociation, and/or increased stability to its target entity as compared with the competing alternative target(s).

Subject: As used herein, the term “subject” refers to an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Therapeutic agent: As used herein, the phrase “therapeutic agent” in general refers to any agent that elicits a desired pharmacological effect when administered to an organism. In some embodiments, an agent is considered to be a therapeutic agent if it demonstrates a statistically significant effect across an appropriate population. In some embodiments, the appropriate population may be a population of model organisms. In some embodiments, an appropriate population may be defined by various criteria, such as a certain age group, gender, genetic background, preexisting clinical conditions, etc. In some embodiments, a therapeutic agent is a substance that can be used to alleviate, ameliorate, relieve, inhibit, prevent, delay onset of, reduce severity of, and/or reduce incidence of one or more symptoms or features of a disease, disorder, and/or condition. In some embodiments, a “therapeutic agent” is an agent that has been or is required to be approved by a government agency before it can be marketed for administration to humans. In some embodiments, a “therapeutic agent” is an agent for which a medical prescription is required for administration to humans.

Treat: As used herein, the terms “treat,” “treatment,” or “treating” refer to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example, for the purpose of decreasing the risk of developing pathology associated with the disease, disorder, and/or condition. In some embodiments, treatment may be administered to a subject to prevent the risk of developing pathology associated with or resulting from a medical procedure and/or treatment.

### DETAILED DESCRIPTION OF CERTAIN EMBODIMENTS

### Programmed Axonal Degeneration

Axonal degeneration is a major pathological feature of neurological diseases such as, but not limited to, Alzheimer's disease, Parkinson's disease, ALS, multiple sclerosis, diabetic peripheral neuropathy, chemotherapy-induced peripheral neuropathy, inherited neuropathy, traumatic brain injury, and/or glaucoma. Damaged or unhealthy axons are eliminated via an intrinsic self-destruction program known as Wallerian degeneration that is distinct from traditional cellular death pathways like apoptosis (Gerdts, J., et al., Neuron, 2016, 89, 449-460; Whitmore, A. et al., Cell Death Differ., 2003, 10, 260-261, each of which is hereby incorporated by reference in its entirety). During Wallerian degeneration, a nerve undergoes selective breakdown of the axon segment distal to an injury, whereas the proximal axon segment and cell body remain intact. Axonal degeneration following an injury is characterized by the sequential depletion of NMNAT2, NAD+ and ATP, followed by neurofilament proteolysis and axonal fragmentation occurring approximately 8 to 24 hours after the original injury (Gerdts, J., et al., Neuron, 2016, 89, 449-460, hereby incorporated by reference in its entirety).

The discovery of the Wallerian degeneration slow (Wlds) protein, which dramatically delays axon degeneration after injury, raised hopes that blocking Wallerian degeneration would be useful in the treatment of neurological disorders (Conforti et al., Nat Rev Neurosci. 2014, 15(6), 394-409; Mack et al., Nat Neurosci. 2001, 4(12), 1199-1206, each of which is hereby incorporated by reference in its entirety). The Wlds protein blocks axon degeneration by mislocalizing the nuclear nicotinamide adenine dinucleotide (NAD+) biosynthetic enzyme NMNAT1 into axons, thereby substituting for the loss of the labile axon maintenance factor NMNAT2 and preventing the NAD+ degradation following an injury (Araki et al., Science. 2004, 305(5686), 1010-1013; Babetto et al., J Neurosci., 2010, 30(40), 13291-13304; Gilley et al., PLoS Biol. 2010, 8(1), e1000300; Sasaki et al., J Biol Chem., 2010, 285(53), 41211-41215, each of which is hereby incorporated by reference in its entirety). These results highlighted the importance of NAD+ in the maintenance of axonal integrity.

NAD+ is a natural coenzyme that functions as an intermediary in cellular oxidation and reduction reactions as well as an ADP-ribosyltransferase substrate. NAD+ has critical roles in energy metabolism, ATP synthesis and cellular signaling (Belenkey et al., Trends Biochem., 2007, 32, 12-19; Chiarugi et al., Nat. Rev. Cancer, 2012, 12, 741-752, each of which is hereby incorporated by reference in its entirety). Increasing intracellular NAD+ levels can improve the health of a cell. Furthermore, the homeostatic regulation of NAD+ levels is responsible for maintaining axonal stability and integrity. Accordingly, manipulations that increase axonal localization of NMNAT, the nicotinamide adenine dinucleotide (NAD+) biosynthetic enzyme, confer axonal protection (Babetto et al., Cell Rep., 2010, 3, 1422-1429; Sasaki et al., J. Neurosci., 2009, each of which is hereby incorporated by reference in its entirety). Exogenous application of the NAD+ precursors that are the substrates of these enzymes, including nicotinic acid mononucleotide, nicotinamide mononucleotide, and nicotinamide riboside (NR), can also delay axonal degeneration (Sasaki et al., J. Neurosci, 2006, 26(33):8481-8491, which is hereby incorporated by reference in its entirety).

NR is one NAD+ precursor which is converted to NAD+ in mammals (Bieganowski and Brenner, Cell., 2004, 117(4), 495-502; Bieganowski and Brenner, J Biol Chem., 2003, 278(35), 33056-33059, each of which is hereby incorporated by reference in its entirety). NR has been found to protect damaged neurons as well as affect cognition in transgenic mouse models of Alzheimer's disease. In Tg2576 mice, which overexpress amyloid precursor protein (APP), chronic NR application increased cognitive performance in aged mice (Gong et al. Neurobiol Aging, 2013, 34(6): 1581-1588, which is hereby incorporated by reference in its entirety). By treating 7-8 month old Tg2576 mice were treated with 250 mg/kg/day of NR (equivalent to 1300 mg/kg/day in the human) for 3 months via drinking water, Gong and colleagues found that NR treatment significantly improved the cognitive performance of Tg2576 mice in an object recognition test, a hippocampal- and cortical-dependent learning task. Whereas NR-treated Tg2576 mice recognizing a novel level object performed significantly better than chance, non-treated, control Tg2576 mice performed at the chance level. In addition to NR, other factors that interfere with the NAD+ biosynthetic pathway or otherwise promote or maintain NAD+ levels have been found to affect neuronal or axonal survival.

It has also been recently discovered that knocking-down or eliminating the expression of SARM1 leads to long-lasting protection of sensory neurons against injury-induced axonal degeneration (Gerdts et al., J. Neurosci, 2013, 33, 13569-13580, which is hereby incorporated by reference in its entirety). Following axonal damage, SARM1 serves as the central executioner in the axonal degeneration pathway. Activated SARM1 is a highly effective NADase that depletes local axonal NAD+ reserves within minutes to a few hours after activation, leading to a local bioenergetic crisis, followed by rapid axonal degeneration. SARM1 belongs to the myeloid differentiation primary response 88 (MYD88)-cytosolic adaptor protein family. However, SARM1 is unique among this family because it is the most evolutionary ancient adaptor, paradoxically inhibits TLR signaling, and has been identified as the central executioner of the injury-induced axon death pathway (O'Neill, L. A. & Bowie, A. G., Nat. Rev. Immunol., 2007, 7, 353-364; Osterloh, J. M., et al., Science, 2012, 337, 481-484; Gerdts, J., et al., J. Neurosci. 33, 2013, 13569-13580, each of which is hereby incorporated by reference in its entirety). Activation of SARM1 via axonal injury or forced dimerization of SARM1-TIR domains promotes rapid and catastrophic depletion of Nicotinamide Adenine Dinucleotide (NAD+), followed soon after by axonal degeneration, thus highlighting the central role of NAD+ homeostasis in axonal integrity (Gerdts, J., et al., Science, 2015, 348, 453-457). SARM1 is required for this injury-induced NAD+ depletion both in vitro and in vivo and SARM1 activation triggers axon degeneration locally via NAD+ destruction (Gerdts et al., et al., Science, 2015 348, 452-457; Sasaki et al., J. Biol. Chem. 2015, 290, 17228-17238, each of which is hereby incorporated by reference in its entirety).

Genetic loss-of-function studies indicate that SARM1 serves as the central executioner of the axonal degeneration pathway following an injury. Genetic deletion or knockout of SARM1 allows for preservation of axons for up to 14 days after nerve transection (Osterloh, J. M., et al., Science, 2012, 337, 481-484; Gerdts, J., et al. J. Neurosci., 2013, 33, 13569-13580, each of which is hereby incorporated by reference in its entirety) and also improves functional outcomes in mice after traumatic brain injury (Henninger, N. et al., Brain, 139, 2016, 1094-1105, which is hereby incorporated by reference in its entirety). In addition to the direct role SARM1 has in axonal injury, SARM1 is also required for the axonal degeneration observed in chemotherapy-induced peripheral neuropathy (CIPN). Loss of SARM1 blocks CIPN, both inhibiting axonal degeneration and heightened pain sensitivity that develops after chemotherapeutic vincristine treatment (Geisler et al, Brain, 2016, 139, 3092-3108, which is hereby incorporated by reference in its entirety).

SARM1 contains multiple conserved motifs including SAM domains, ARM/HEAT motifs and a TIR domain that mediate oligomerization and protein-protein interactions (O'Neill, L. A. & Bowie, A. G., Nat. Rev. Immunol., 2007, 7, 353-364; Tewari, R., et al., Trends Cell Biol., 2010, 20, 470-481; Qiao, F. & Bowie, J. U., Sci. STKE 2005, re7, 2005, each of which is hereby incorporated by reference in its entirety). TIR domains are commonly found in signaling proteins functioning in innate immunity pathways where they serve as scaffolds for protein complexes (O'Neill, L. A. & Bowie, A. G., Nat. Rev. Immunol., 2007, 7, 353-364). Interestingly, dimerization of SARM1-TIR domains is sufficient to induce axonal degeneration and to rapidly trigger degradation of NAD+ by acting as the NAD+ cleaving enzyme (Milbrandt et al., WO 2018/057989; Gerdts, J., et al., Science, 2015, 348, 453-457, each of which is hereby incorporated by reference in its entirety). Given the central role of SARM1 in the axonal-degeneration pathway and its identified NADase activity, efforts have been undertaken to identify agents that can regulate SARM1, and potentially act as useful therapeutic agents, for example, to protect against neurodegenerative diseases including peripheral neuropathy, traumatic brain injury, and/or neurodegenerative diseases. SARM1-dependent NAD+ consumption is the central biochemical event in the axonal degeneration program.

Among other things, the present disclosure provides methods for inhibiting SARM1. Among other things, the present disclosure provides a combination of a SARM1 inhibitor and NAD+ or a NAD+ precursor (e.g., NR, NA, NaR, NAM, NMN, NaMN, TRP, vitamin B₃, or NAAD) for use in stabilizing neurons whose axons have been injured. In some embodiments, such combinations allow the axons to be repaired rather than degenerate.

### Methods of Treating Neurodegeneration

In some embodiments, NAD+ or a NAD+ precursor (e.g., NR, NA, NaR, NAM, NMN, NaMN, TRP, vitamin B₃, or NAAD) stimulates or results in an increase in NAD+ concentration. In some embodiments, the present disclosure provides NAD+ or a NAD+ precursor (e.g., NR, NA, NaR, NAM, NMN, NaMN, TRP, vitamin B₃, or NAAD) in combination with a SARM1 inhibitor. In some embodiments a NAD+ precursor is one or more compounds described herein (e.g., NR, NA, NaR, NAM, NMN, NaMN, TRP, vitamin B₃, or NAAD).

Nicotinamide adenine dinucleotide (NAD) is a coenzyme found in all living cells. Nicotinamide adenine dinucleotide exists in two forms: an oxidized and reduced form abbreviated as NAD+ and NADH, respectively. In some embodiments, NAD+ and NADH are in a phosphorylated form: NADP and NADPH, respectively. NAD+ and NADH function as coenzymes in a wide variety of enzymatic oxidation-reduction reactions essential for tissue respiration, lipid metabolism, and glycogenolysis.

Nicotinamide riboside (NR) is a pyridine-nucleoside form of vitamin B₃ that functions as a precursor to nicotinamide adenine dinucleotide (NAD+). In some embodiments, NR is provided as a safe vitamin B₃ isoform used as dietary supplement, with a Generally Recognized As Safe (GRAS) designation from the FDA.

Nicotinic acid (NA), also known as niacin, is a form of vitamin B₃. In some embodiments, NA is provided as a safe vitamin B₃ isoform used as dietary supplement. In some embodiments, NA is provided as a synthetic prodrug, e.g., myristyl nicotinic acid (MNa).

Nicotinic acid riboside (NaR) is a precursor to nicotinamide riboside.

Nicotinamide (NAM) is a water-soluble component of the vitamin B complex group. In some embodiments, NAM is provided as dietary supplement.

Nicotinamide mononucleotide (NMN) is a derivative of niacin that can be enzymatically converted to nicotinamide adenine dinucleotide (NAD). In some embodiments, NMN is provided as a dietary supplement.

Nicotinic acid mononucleotide (NaMN) is formed in the first step of Preiss-Handler pathway for the biosynthesis of NAD+. In some embodiments, NaMN is provided as a dietary supplement.

Tryptophan (TRP) is an α-amino acid that is used in the biosynthesis of proteins. TRP also functions as a biochemical precursor for niacin. In some embodiments, TRP is provided as a dietary supplement.

Nicotinic acid adenine dinucleotide (NAAD) is a Ca²⁺-mobilizing second messenger synthesized in response to extracellular stimuli. In some embodiments, NAAD is in a phosphorylated form: nicotinic acid adenine dinucleotide phosphate (NAADP). In some embodiments, NAAD is part of the nicotinate and nicotinamide metabolic pathway.

For the avoidance of doubt, the structures of NA, NAM, NaR, NR, NaMN, NMN, NAD and NAAD are set forth below:

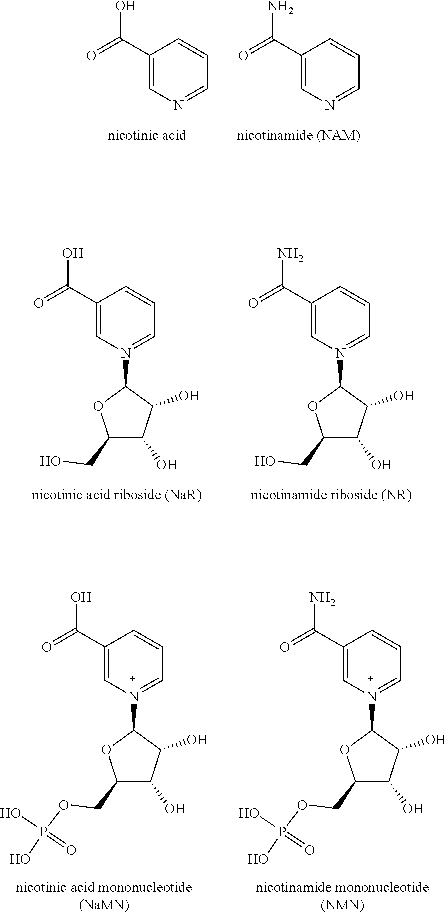

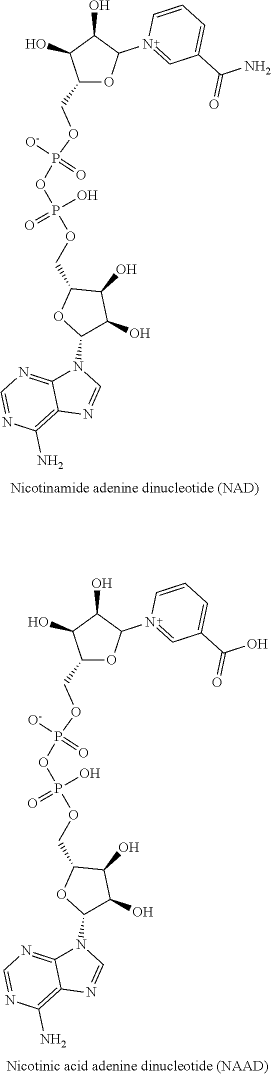

In some embodiments, the present disclosure provides a method for treating subjects suffering from one or more diseases, disorders, or conditions. In some embodiments, the one or more diseases, disorders, or conditions are mediated by SARM1.

In some embodiments, the one or more diseases, disorders, or conditions is/are acute. In some embodiments, the one or more diseases, disorders, or conditions is/are chronic.

In some embodiments, the one or more diseases, disorders, or conditions is/are characterized by axonal degeneration in the central nervous system, the peripheral nervous system, the optic nerve, the cranial nerves, or a combination thereof.

In some embodiments, provided combination therapies and methods promote the increase of intracellular levels of nicotinamide adenine dinucleotide (NAD+) in cells and tissues for improving cell and tissue survival. In some embodiments, provided combination therapies methods increase NAD+ levels in cells and tissues. In some embodiments, provided combination therapies and methods improve cell and tissue survival. In some embodiments, provided combination therapies and methods stabilize the neurons and/or cells until the external environment stabilizes following an acute event.

In some embodiments, the present disclosure provides a method for treating, preventing, and/or ameliorating a neurodegenerative disease, disorder or condition comprising administering a SARM1 inhibitor and NAD+ or a NAD+ precursor (e.g., NR, NA, NaR, NAM, NMN, NaMN, TRP, vitamin B₃, or NAAD). In some embodiments, a neurodegenerative disease, disorder or condition is associated with axonal degeneration. Accordingly, in some embodiments, the present disclosure provides a method of for treating, preventing, and/or ameliorating axonal degeneration comprising administering to a subject in need thereof a SARM1 inhibitor in combination with NAD+ or a NAD+ precursor (e.g., NR, NA, NaR, NAM, NMN, NaMN, TRP, vitamin B₃, or NAAD).

In some embodiments, provided combination therapies and/or methods prevent or slow the degeneration of a neuron, a part of an intact neuron, or a cellular fragment derived from a neuron. In some embodiments, provided combinations and/or methods prevent or slow the progression of degeneration of the portion of the axon distal to an axonal injury. In some embodiments, provided methods and/or combinations, as described herein, are useful as stabilizing agents to promote neuronal survival. In some embodiments, provided combination therapies are useful for maintaining the function of an axon including, but not limited to, metabolism, axonal integrity, intracellular transport, and action potential propagation.

In some embodiments, provided methods treat or prevent secondary conditions associated with neurodegenerative disorders. Such secondary conditions include, but not limited to, muscle impairments, respiratory impairments, anxiety, depression, speech impairments, pulmonary embolisms, cardiac arrhythmias, and/or pneumonia.

In some embodiments, the present disclosure relates to a method of treating, preventing, and/or ameliorating a neurodegenerative disease, disorder or condition comprising i) providing a) a subject diagnosed with, at risk for, or exhibiting symptoms of, a neurodegenerative disease, disorder or condition and b) a combination comprising a SARM1 inhibitor and NAD+ or a NAD+ precursor (e.g., NR, NA, NaR, NAM, NMN, NaMN, TRP, vitamin B₃, or NAAD); and ii) administering said combination to said subject under conditions such that said neurodegenerative disease, disorder or condition is reduced.

In some embodiments, the present disclosure provides a combination therapy comprising a SARM1 inhibitor and NAD+ or a NAD+ precursor (e.g., NR, NA, NaR, NAM, NMN, NaMN, TRP, vitamin B₃, or NAAD). In some embodiments, provided combination therapies comprise a SARM1 inhibitor, NAD+ or a NAD+ precursor (e.g., NR, NA, NaR, NAM, NMN, NaMN, TRP, vitamin B₃, or NAAD), and one or more additional therapeutic agents.

As used herein, the term NAD+ precursor refers to a compound that may participate in the NAD+ metabolic pathway. In some embodiments, NAD+ or a NAD+ precursor (e.g., NR, NA, NaR, NAM, NMN, NaMN, TRP, vitamin B₃, or NAAD) stimulates an increase in NAD+ concentration. In some embodiments, a NAD+ precursor is vitamin B₃. In some embodiments, a NAD+ precursor is a form of vitamin B₃. In some embodiments, a NAD+ precursor is nicotinamide riboside (NR), also known as 1-(β-D-ribofuranosyl)nicotinamide or N-ribosylnicotinamide. In some embodiments, a NAD+ precursor is nicotinic acid (NA), also known as niacin. In some embodiments, a NAD+ precursor is nicotinic acid riboside (NaR). In some embodiments a NAD+ precursor is nicotinamide (NAM), also known as 3-pyridinecarboxamide, niacinamide, nicotinic acid amide, or nicotinic amide. In some embodiments, a NAD+ precursor is nicotinamide mononucleotide (NMN), also known as, nicotinamide ribonucleoside 5′-phosphate, nicotinamide D-ribonucleotide, β-nicotinamide ribose monophosphate, or nicotinamide nucleotide. In some embodiments, a NAD+ precursor is nicotinic acid mononucleotide (NaMN). In some embodiments, a NAD+ precursor is tryptophan (TRP), also known as (2S)-2-amino-3-(1H-indol-3-yl)propanoic acid or 2-amino-3-(1H-indol-3-yl)propanoic acid. In some embodiments, a NAD+ precursor is deamido-NAD+, also known as deamido-NAD, deamino-NAD+, Nicotinic acid adenine dinucleotide (NAAD). In some embodiments a NAD+ precursor is nicotinic acid riboside, O-ethylnicotinate riboside, or O-methylnicotinate riboside (Yang et al., J. Med. Chem., 2007, 50 (26), 6458-6461). In some embodiments, a NAD+ precursor is a β-nicotinamide riboside. In some embodiments, a NAD+ precursor is a nicotinate ester nucleoside derivative. In some embodiments, a NAD+ precursor is a triacetyl-O-ethylnicotinate riboside (Yang et al., J. Med. Chem., 2007, 50 (26), 6458-6461).

In some embodiments, a provided combination therapy comprises a SARM1 inhibitor, NAD+ or a NAD+ precursor (e.g., NR, NA, NaR, NAM, NMN, NaMN, TRP, vitamin B₃, or NAAD), and one or more additional therapeutic agents. In some embodiments, the one or more additional therapeutic agents is/are selected from acetylcholine esterase inhibitors, NMDA agonists, Donepezil, Galantamine, Memantine, Rivastigmine, rilzuole, edaravone, levodopa, carbidopa, anticholinergics, bromocriptine, pramipexole, ropinirole, and/or amantadine. In some embodiments, the one or more additional therapeutic agents is/are selected from immunosuppressive drugs such as prednisone, cyclosporine, or azathioprine, and nonsteroidal anti-inflammatory drugs (NSAIDs). In some embodiments, the one or more additional therapeutic agents include antidepressants, anticonvulsants, antiarrythmics (e.g., mexiletine), and narcotic agents, tricyclic antidepressants such as amitriptyline or newer serotonin-norepinephrine reuptake inhibitors such as duloxetine hydrochloride or venlafaxine. In some embodiments anticonvulsants are one of the following: gabapentin, pregabalin, topiramate, and carbamazepine. In some embodiments, the one or more additional therapeutic agents combined with the present disclosure include anti-epileptic treatments. In some embodiments, the one or more additional therapeutic agents is intravenous immuonoglobin (IV Ig). In some embodiments, the one or more additional therapeutic agents is/are selected from multiple sclerosis disease-modifying therapeutics (DMTs) including, but not limited to, interferon beta-1a, interferon beta-1b, glatiramer acetate, daclizumab, teriflunomide, fingolimod, dimethyl fumarate, alemtuzumab, mitoxantrone, ocrelizumab, and natalizumab.

In some embodiments, such combination therapies are useful for treating, preventing, and/or ameliorating a neurodegenerative disease, disorder or condition. In some embodiments, provided combination therapies are useful for treating, preventing, and/or ameliorating axonal degeneration. In some embodiments, provided combination therapies are useful for preventing or slowing the progression of degeneration of the axon distal to an axonal injury.

In some embodiments, a neurodegenerative disease, disorder or condition is characterized by axons that are susceptible to disruption or pathologic stress. Such diseases or conditions include, but are not limited to, cancer, diabetes, neurodegenerative diseases, cardiovascular disease, blood clotting, inflammation, flushing, obesity, aging, or stress.

In some embodiments, a neurodegenerative disease, disorder or condition is selected from the group consisting of a neuropathy or an axonopathy. In some embodiments, an axonopathy or a neuropathy is any disease, disorder or condition involving neurons and/or supporting cells, such as for example, glia, muscle cells or fibroblasts, and, in particular, those diseases or conditions involving axonal damage. Axonal damage can be caused by traumatic injury or by non-mechanical injury due to diseases, conditions, or exposure to toxic molecules or drugs. The result of such damage can be degeneration or dysfunction of the axon and loss of functional neuronal activity. Disease and conditions producing or associated with such axonal damage are among a large number of neuropathic diseases and conditions. Such neuropathies can include peripheral neuropathies, central neuropathies, and combinations thereof. Furthermore, peripheral neuropathic manifestations can be produced by diseases focused primarily in the central nervous systems and central nervous system manifestations can be produced by essentially peripheral or systemic diseases.

In some embodiments, a neurodegenerative disease, disorder or condition may be a traumatic neuronal injury. In some embodiments, injury to the spinal cord and/or traumatic brain injury. In some embodiments, a traumatic neuronal injury is blunt force trauma, a closed-head injury, an open head injury, exposure to a concussive and/or explosive force, a penetrating injury in or to the brain cavity or innervated region of the body. In some embodiments, a traumatic neuronal injury is a force which causes axons to deform, stretch, crush or sheer. In some embodiments, a neurodegenerative disease, disorder or condition is an acute injury to the central nervous system. In some embodiments, the condition is or comprises a chronic injury to the central nervous system, e.g., injury to the spinal cord, traumatic brain injury, and/or traumatic axonal injury. In some embodiments, the condition is or comprises chronic traumatic encephalopathy (CTE). In some embodiments, a traumatic neuronal injury results from increased intraocular pressure.

In some embodiments, the neurodegenerative or neurological disease, disorder or condition is associated with axonal degeneration, axonal damage, axonopathy, a demyelinating disease, a central pontine myelinolysis, a nerve injury disease, disorder or condition, a metabolic disease, a mitochondrial disease, metabolic axonal degeneration, axonal damage resulting from a leukoencephalopathy or a leukodystrophy.

In some embodiments, a neuropathy or axonopathy is associated with axonal degeneration. In some embodiments, a neuropathy associated with axonal degeneration is a hereditary or congenital neuropathy or axonopathy. In some embodiments, a neuropathy associated with axonal degeneration results from a de novo or somatic mutation. In some embodiments, a neuropathy associated with axonal degeneration results from idiopathic conditions.

In some embodiments, provided methods as described herein are useful, for example for inhibiting or preventing degeneration of a central nervous system (neuron) or a portion thereof. In some embodiments, the present disclosure provides a combination therapy comprising a SARM1 inhibitor and NAD+ or a NAD+ precursor (e.g., NR, NA, NaR, NAM, NMN, NaMN, TRP, vitamin B₃, or NAAD) that is useful, for example as a method for inhibiting the degeneration of a peripheral nervous system neuron or a portion thereof.

In some embodiments, a peripheral neuropathy can involve damage to the peripheral nerves, and/or can be caused by diseases of the nerves or as the result of systemic illnesses. Some such diseases can include diabetes, uremia, infectious diseases such as AIDS or leprosy, nutritional deficiencies, vascular or collagen disorders such as atherosclerosis, and autoimmune diseases such as systemic lupus erythematosus, scleroderma, sarcoidosis, rheumatoid arthritis, and polyarteritis nodosa. In some embodiments, peripheral nerve degeneration results from traumatic (mechanical) damage to nerves as well as chemical or thermal damage to nerves. Such conditions that injure peripheral nerves include compression or entrapment injuries such as carpal tunnel syndrome, direct trauma, penetrating injuries, contusions, fracture or dislocated bones; pressure involving superficial nerves (ulna, radial, or peroneal) which can result from prolonged use of crutches or staying in one position for too long, or from a tumor; intraneural hemorrhage; ischemia; exposure to cold or radiation or certain medicines or toxic substances such as herbicides or pesticides. In particular, the nerve damage can result from chemical injury due to a cytotoxic anticancer agent such as, for example, taxol, cisplatinin, a proteasome inhibitor, or a vinca alkaloid such as vincristine. Typical symptoms of such peripheral neuropathies include weakness, numbness, paresthesia (abnormal sensations such as burning, tickling, pricking or tingling) and pain in the arms, hands, legs and/or feet. In some embodiments, a neuropathy is associated with mitochondrial dysfunction. Such neuropathies can exhibit decreased energy levels, i.e., decreased levels of NAD+ and ATP.

In some embodiments neurodegenerative diseases, disorders, or conditions that are associated with neuropathy or axonopathy in the central nervous system include diseases involving progressive dementia such as, for example, Alzheimer's disease, senile dementia, Pick's disease, and Huntington's disease; central nervous system diseases affecting muscle function such as, for example, Parkinson's disease, motor neuron diseases, progressive ataxias, and amyotrophic lateral sclerosis; demyelinating diseases such as, for example multiple sclerosis. Mechanical injuries or traumatic injuries to the head and spine can also cause nerve injury and degeneration in the brain and spinal cord. In some embodiments, ischemia and/or stroke as well as conditions such as nutritional deficiency and chemical toxicity such as with chemotherapeutic agents can cause central nervous system neuropathies.

In some embodiments, a neuropathy or axonopathy associated with axonal degeneration, includes, but is not limited to, Parkinson's disease, Alzheimer's disease, Huntington's disease, Herpes infection, diabetes, amyotrophic lateral sclerosis (ALS), a demyelinating disease, ischemia or stroke, frontotemporal dementia, ataxias, Charcot Marie Tooth, neuromyelitis optica, traumatic brain injury, chemical injury, thermal injury, and AIDS.

In some embodiments, subjects to which a combination therapy as described herein is administered are subjects suffering from or susceptible to a neurodegenerative disease, disorder or condition. In some embodiments, the subject is at risk of developing a neurodegenerative disease, disorder or condition. In some embodiments, the present disclosure provides a method comprising administering to a subject at risk for developing a neurodegenerative disease, disorder or condition a SARM1 inhibitor in combination with NAD+ or a NAD+ precursor (e.g., NR, NA, NaR, NAM, NMN, NaMN, TRP, vitamin B₃, or NAAD). In some embodiments, the neurodegenerative disease, disorder or condition is characterized by axonal degeneration

In some embodiments, the neurodegenerative or neurological disease, disorder or condition is selected from the group consisting of spinal cord injury, stroke, multiple sclerosis, progressive multifocal leukoencephalopathy, congenital hypomyelination, encephalomyelitis, acute disseminated encephalomyelitis, central pontine myelolysis, osmotic hyponatremia, hypoxic demyelination, ischemic demyelination, adrenoleukodystrophy, Alexander's disease, Niemann-Pick disease, Pelizaeus Merzbacher disease, periventricular leukomalacia, globoid cell leukodystrophy (Krabbe's disease), Wallerian degeneration, optic neuritis, transverse myelitis, amyotrophic lateral sclerosis (ALS, Lou Gehrig's disease), Huntington's disease, Alzheimer's disease, Parkinson's disease, Tay-Sachs disease, Gaucher's disease, Hurler Syndrome, traumatic brain injury, post radiation injury, neurologic complications of chemotherapy (chemotherapy induced neuropathy; CIPN), neuropathy, acute ischemic optic neuropathy, vitamin B12 deficiency, isolated vitamin E deficiency syndrome, Bassen-Kornzweig syndrome, Glaucoma, Leber's hereditary optic atrophy (neuropathy), Leber congenital amaurosis, neuromyelitis optica, metachromatic leukodystrophy, acute hemorrhagic leukoencephalitis, trigeminal neuralgia, Bell's palsy, cerebral ischemia, multiple system atrophy, traumatic glaucoma, tropical spastic paraparesis human T-lymphotropic virus 1 (HTLV-1) associated myelopathy, west Nile virus encephalopathy, La Crosse virus encephalitis, Bunyavirus encephalitis, pediatric viral encephalitis, essential tremor, Charcot-Marie-Tooth disease, motor neuron disease, spinal muscular atrophy (SMA), hereditary sensory and autonomic neuropathy (HSAN), adrenomyeloneuropathy, progressive supra nuclear palsy (PSP), Friedrich's ataxia, hereditary ataxias, noise induced hearing loss, congenital hearing loss, age-related hearing loss, Lewy Body Dementia, frontotemporal dementia, amyloidosis, diabetic neuropathy, HIV neuropathy, enteric neuropathies and axonopathies, Guillain-Barre syndrome, severe acute motor axonal neuropathy (AMAN), Creutzfeldt-Jakob disease, transmissible spongiform encephalopathy, spinocerebellar ataxias, pre-eclampsia, hereditary spastic paraplegias, spastic paraparesis, familial spastic paraplegia, French settlement disease, Strumpell-Lorrain disease, and non-alcoholic steatohepatitis (NASH).

In some embodiments, a neurodegenerative disease, disorder or condition includes conditions producing or associated with neuronal or axonal damage. Such neurodegenerative diseases, disorders or conditions can include a peripheral neuropathy, a central neuropathy, or a combination thereof. In some embodiments, a peripheral neuropathy can be produced by a disease focused primarily in the central nervous systems and a central nervous system neuropathy can be produced by essentially peripheral or systemic diseases.

In some embodiments, the neurodegenerative disease, disorder or condition is an acute peripheral neuropathy. In some embodiments an acute peripheral neuropathy is a chemotherapy-induced peripheral neuropathy (CIPN). CIPN can be induced by various drugs, such as, but not limited to, thalidomide, epothilones (e.g., ixabepilone), taxanes (e.g., paclitaxel and docetaxel), vinca alkaloids (e.g., vinblastine, vinorelbine, vincristine, and vindesine), proteasome inhibitors (e.g., bortezomib), platinum-based drugs (e.g., cisplatin, oxaliplatin, and carboplatin) and auristatins (e.g., conjugated monomethyl auristatin E).

In some embodiments, the present disclosure provides methods of treating, preventing, and/or ameliorating neurodegenerative or neurological diseases or conditions related to axonal degeneration, axonal damage, axonopathies, demyelinating diseases, central pontine myelinolysis, nerve injury diseases or disorders, metabolic diseases, mitochondrial diseases, metabolic axonal degeneration, axonal damage resulting from a leukoencephalopathy or a leukodystrophy. In some embodiments, the axonal degeneration results from reduction or depletion of NAD+.

In some embodiments, a neurodegenerative disease, disorder or condition is a central nervous system disease or disorder, a peripheral neuropathy or disorder, a disorder of the optic nerve, a metabolic disorder, a traumatic injury, viral encephalitides, exposure to toxic molecules or drugs, a neuropathy associated with pain. In some embodiments, viral encephalitides include those caused by enteroviruses, arboviruses, herpes simplex virus. In some embodiments viral encephalitides include West Nile virus encephalitis, La Crosse virus encephalitis, Bunyavirus encephalitis, pediatric viral encephalitis, and AIDS dementia complex (also known as HIV dementia, HIV encephalopathy, and HIV-associated dementia).

In some embodiments, a neurodegenerative disease, disorder or condition is associated with conditions that produce pain. Pain neuropathies that can be treated according to the methods of the disclosure include those associated with the following conditions: chronic pain, fibromyalgia, spinal pain, carpal tunnel syndrome, pain from cancer, arthritis, sciatica, headaches, pain from surgery, muscle spasms, back pain, visceral pain, pain from injury, dental pain, neuralgia, such as neurogenic or neuropathic pain, nerve inflammation or damage, shingles, herniated disc, torn ligament, and diabetes.

In some embodiments, a neurodegenerative disease, disorder or condition affects the central nervous system. In some embodiments a neurodegenerative disease, disorder or condition includes, but is not limited to, Alzheimer's disease, Parkinson's disease, amyotrophic lateral sclerosis (ALS, Lou Gehrig's disease), multiple sclerosis, Huntington's disease, senile dementia, Pick's disease, Tay-Sachs disease, motor neuron disease, ataxia, spinal muscular atrophy (SMA), Bassen-Kornzweig syndrome, Charcot-Marie-Tooth disease, motor neuron disease, hereditary sensory and autonomic neuropathy (HSAN), adrenomyeloneuropathy, progressive supra nuclear palsy (PSP), and/or Friedrich's ataxia.

In some embodiments, a neurodegenerative disease, disorder or condition affects the peripheral nervous system. In some embodiments, a peripheral neuropathy can involve damage to the peripheral nerves, and/or can be caused by diseases of the nerves or as the result of systemic illnesses. In some embodiments, a peripheral neuropathy is selected from diabetes, uremia, infectious diseases such as AIDS or leprosy, nutritional deficiencies, vascular or collagen disorders such as atherosclerosis, and autoimmune diseases such as systemic lupus erythematosus, scleroderma, sarcoidosis, rheumatoid arthritis, and polyarteritis nodosa.

In some embodiments, a neurodegenerative disease, disorder or condition affects the optic nerve. In some embodiments, the condition is an acute condition affecting the optic nerve, for example, but not limited to, acute optic neuropathy (AON) or acute angle closure glaucoma. In some embodiments, the condition is a genetic or idiopathic retinal condition. In some embodiments, the condition increases intraocular pressure, such as, for example, increased intraocular pressure leading to glaucoma. In some embodiments, a neurodegenerative disease, disorder or condition is a genetic or idiopathic retinal condition, such as that resulting in axonal degeneration of, e.g., the optic nerve, resulting in loss of vision. In some embodiments, the condition is a chronic condition affecting the optic nerve, for example, but not limited to, Leber's congenital amaurosis, Leber's hereditary optic neuropathy, primary open angle glaucoma, and autosomal dominant optic atrophy.

In some embodiments, optic nerve neuropathies include, but are not limited to, glaucoma; retinal ganglion degeneration such as those associated with retinitis pigmentosa and outer retinal neuropathies; optic nerve neuritis and/or degeneration including that associated with multiple sclerosis. In some embodiments an optic neuropathy neurotraumatic injury to the optic nerve which can include, for example, injury during tumor removal. In some embodiments, an optic nerve neuropathy is a hereditary optic neuropathies such as Kjer's disease and Leber's hereditary optic neuropathy; ischemic optic neuropathies, such as those secondary to giant cell arteritis; metabolic optic neuropathies such as neurodegenerative diseases including Leber's neuropathy, nutritional deficiencies such as deficiencies in vitamins B12 or folic acid, and toxicities such as due to ethambutol or cyanide; neuropathies caused by adverse drug reactions and neuropathies caused by vitamin deficiency. Ischemic optic neuropathies also include non-arteritic anterior ischemic optic neuropathy.

In some embodiments, a neurodegenerative disease, disorder or condition is a peripheral neuropathy or peripheral nervous system disorder. In some embodiments, peripheral neuropathy is a metabolic and endocrine neuropathy which includes a wide spectrum of peripheral nerve disorders associated with systemic diseases of metabolic origin. Such diseases and disorders include, for example, diabetes mellitus, hypoglycemia, uremia, hypothyroidism, hepatic failure, polycythemia, amyloidosis, acromegaly, porphyria, disorders of lipid/glycolipid metabolism, nutritional/vitamin deficiencies, and mitochondrial disorders, among others. In some embodiments these peripheral nerve disorders can be identified by the involvement of peripheral nerves by alteration of the structure or function of myelin and axons due to metabolic pathway dysregulation.

In some embodiments, the subject is at risk of developing a condition characterized by axonal degeneration. In some embodiments, the subject is identified as being at risk of axonal degeneration, e.g., based on the subject's genotype, a diagnosis of a condition associated with axonal degeneration, and/or exposure to an agent and/or a condition that induces axonal degeneration.

In some embodiments, the subject has a condition characterized by axonal degeneration. In some embodiments, the subject has been diagnosed with a condition characterized by axonal degeneration.

In some embodiments, a combination therapy provided herein is characterized such that, when administered to a population of subjects, the combination therapy reduces one or more symptoms or features of neurodegeneration. For example, in some embodiments, a relevant symptom or feature may be selected from the group consisting of extent, rate, and/or timing of neuronal disruption.

In some embodiments, the subject engages in an activity identified as a risk factor for neuronal degeneration, e.g., a subject that engages in contact sports or occupations with a high chance for traumatic neuronal injury. In some embodiments, the contact sport includes but is not limited to American football, basketball, boxing, diving, field hockey, football, ice hockey, lacrosse, martial arts, rodeo, rugby, ski jumping, water polo, wrestling, baseball, cycling, cheerleading, fencing, track and field, gymnastics, handball, horseback riding, skating, skiing, skateboarding, softball, squash, ultimate Frisbee, volleyball, and/or windsurfing.

In some embodiments, provided methods comprise administering a combination therapy as described herein to a subject population in need thereof. In some embodiments the subject and/or subject population is elderly.

In some embodiments, provided combination therapies are useful, for example, in treating a population at risk of developing a condition characterized by axonal and/or neuronal degeneration. In some embodiments, the population is drawn from individuals who engage in activities where the potential for traumatic neuronal injury is high. In some embodiments, the population is drawn from athletes who engage in contact sports or other high-risk activities. In some embodiments, the subject population is drawn from those who have been a member of the armed forces or a military contractor.

In some embodiments, the subject and/or subject population is known to have a genetic risk factor for neurodegeneration. In some embodiments, the subject and/or subject population has a family history of neurodegenerative disease. In some embodiments, the subject and/or subject population expresses one or more copies of a known genetic risk factor for neurodegeneration. In some embodiments, the subject and/or subject population is drawn from a population with a high incidence of neurodegeneration. In some embodiments, the subject and/or subject population has a hexanucleotide repeat expansion in chromosome 9 open reading frame 72. In some embodiments, the subject and/or subject population has one or more copies of the ApoE4 allele.

In some embodiments, a subject to whom a provided combination therapy is administered exhibits one or more signs or symptoms associated with axonal degeneration. In some embodiments, the subject does not exhibit any signs or symptoms of neurodegeneration.

In some embodiments, the neurodegenerative disease, disorder or condition is selected from the group consisting of neuropathies or axonopathies. In some embodiments, the present disclosure provides a combination therapy comprising a SARM1 inhibitor and NAD+ or a NAD+ precursor (e.g., NR, NA, NaR, NAM, NMN, NaMN, TRP, vitamin B₃, or NAAD) to treat one or more neurodegenerative diseases, disorders or conditions selected from the group consisting of neuropathies or axonopathies. In some embodiments, the present disclosure provides a combination therapy comprising a SARM1 inhibitor and NAD+ or a NAD+ precursor (e.g., NR, NA, NaR, NAM, NMN, NaMN, TRP, vitamin B₃, or NAAD), for example to treat a neuropathy or axonopathy associated with axonal degeneration. In some embodiments, a neuropathy associated with axonal degeneration is a hereditary or congenital neuropathy or axonopathy. In some embodiments, a neuropathy associated with axonal degeneration results from a de novo or somatic mutation. In some embodiments, a neuropathy associated with axonal degeneration results from idiopathic conditions. In some embodiments, a neuropathy associated with axonal degeneration is selected from a list contained herein.

In some embodiments, provided methods reduce one or more symptoms or features of neurodegeneration. For example, in some embodiments, a relevant symptom or feature may be selected from the group consisting of extent, rate, and/or timing of neuronal disruption. In some embodiments, neuronal disruption may be or comprise axonal degeneration, loss of synapses, loss of dendrites, loss of synaptic density, loss of dendritic arborization, loss of axonal branching, loss of neuronal density, loss of myelination, loss of neuronal cell bodies, loss of synaptic potentiation, loss of action-potential potentiation, loss of cytoskeletal stability, loss of axonal transport, loss of ion channel synthesis and turnover, loss of neurotransmitter synthesis, loss of neurotransmitter release and reuptake capabilities, loss of axon-potential propagation, neuronal hyperexitability, and/or neuronal hypoexcitability. In some embodiments, neuronal disruption is characterized by an inability to maintain an appropriate resting neuronal membrane potential. In some embodiments, neuronal disruption is characterized by the appearance of inclusion bodies, plaques, and/or neurofibrillary tangles. In some embodiments, neuronal disruption is characterized by the appearance of stress granules. In some embodiments, neuronal disruption is characterized by the intracellular activation of one or more members of the cysteine-aspartic protease (Caspase) family. In some embodiments, neuronal disruption is characterized by a neuron undergoing programed cell death (e.g. apoptosis, pyroptosis, ferroapoptosis, and/or necrosis) and/or inflammation.

In certain embodiments, a combination comprising a SARM1 inhibitor and NAD+ or a NAD+ precursor (e.g., NR, NA, NaR, NAM, NMN, NaMN, TRP, vitamin B₃, or NAAD) is useful, for example, as an analytical tool, as a probe in biological assays, or as a therapeutic agent in accordance with the present disclosure.

Such combinations provided by this disclosure are also useful for the study of SARM1 NADase function in biological and pathological phenomena and the comparative evaluation of new SARM1 activity inhibitors in vitro or in vivo. In some embodiments, a combination comprising a SARM1 inhibitor and NAD+ or a NAD+ precursor (e.g., NR, NA, NaR, NAM, NMN, NaMN, TRP, vitamin B₃, or NAAD) is useful for studying axonal integrity. In some embodiments, such combinations are useful for studying apoptosis.

In some embodiments, provided combinations are useful for inhibiting the degeneration of a neuron, or a portion thereof. In some embodiments, provided combinations are useful to treat neurons whose axons are injured. In some embodiments, provided combinations are useful for inhibiting the degeneration of a neuron, or a portion thereof in vivo. In some embodiments, provided combinations are useful as stabilizing agents to promote in vitro neuronal survival.

In some embodiments, the present disclosure provides a method for inhibiting the degeneration of neurons derived from a subject comprising administering to the subject a SARM1 inhibitor in combination with NAD+ or a NAD+ precursor (e.g., NR, NA, NaR, NAM, NMN, NaMN, TRP, vitamin B₃, or NAAD).

In some embodiments, provided combinations are useful to treat neurons whose axons are injured.

In some embodiments, the present disclosure relates to a method of increasing intracellular concentrations of NAD+ comprising: contacting a biological sample with a SARM1 inhibitor and NAD+ or a NAD+ precursor (e.g., NR, NA, NaR, NAM, NMN, NaMN, TRP, vitamin B₃, or NAAD).

In some embodiments, the present disclosure provides a combination therapy comprising a SARM1 inhibitor and NAD+ or a NAD+ precursor (e.g., NR, NA, NaR, NAM, NMN, NaMN, TRP, vitamin B₃, or NAAD) that is useful, for example in affecting biomarkers associated with neurodegeneration. In some embodiments, changes in biomarkers can be detected systemically or with a sample of cerebral spinal fluid (CSF), blood, plasma, serum, and/or tissue from a subject. In some embodiments, provided methods described herein can be used to affect a change in the concentration of neurofilament light chain protein (NF-L) and/or neurofilament heavy chain protein (NF-H) contained in the CSF, blood, plasma, serum, and/or tissue of a subject. In some embodiments, provide methods described herein can affect constitutive NAD+ and/or cADPR levels in neurons and/or axons.

In some embodiments, provided methods comprise administering a combination therapy as described herein to a subject or subject population based on the presence or absence of one or more biomarkers. In some embodiments, provided methods further comprise monitoring the level of a biomarker in the subject and/or subject population and adjusting the dosing regimen accordingly.

In some embodiments, provided methods as described herein can affect a detectable change in the levels of one or more neurodegeneration-associated proteins in a subject. Such proteins include, but are not limited to, albumin, amyloid-β (Aβ)38, Aβ40, Aβ42, glial fibrillary acid protein (GFAP), heart-type fatty acid binding protein (hFABP), monocyte chemoattractin protein (MCP)-1, neurogranin, neuron specific enolayse (NSE), soluble amyloid precursor protein (sAPP)α, sAPPP, soluble triggering receptor expressed on myeloid cells (sTREM) 2, phospho-tau, and/or total-tau. In some embodiments, one or more compounds and/or compositions as described herein can affect a change in cytokines and/or chemokines, including, but not limited to, Ccl2, Ccl7, Ccl12, Csf1, and/or 116.

In some embodiments, provided SARM1 inhibitors reduce or inhibit binding of NAD+ by SARM1. In some embodiments, provided SARM1 inhibitors bind to SARM1 within a pocket comprising one or more catalytic residues (e.g., a catalytic cleft of SARM1). In some embodiments, provided SARM1 inhibitors bind to non-catalytic residues. In some embodiments, provided SARM1 inhibitors are allosteric modulators of SARM1 activity. In some embodiments, provided SARM1 inhibitors reduce SARM1 NADase activity. Accordingly, in some embodiments, the present disclosure provides a method of reducing or inhibiting binding of SARM1 by NAD+ comprising administering to a subject in need thereof a combination of a SARM1 inhibitor and NAD+ or a NAD+ precursor (e.g., NR, NA, NaR, NAM, NMN, NaMN, TRP, vitamin B₃, or NAAD).

In some embodiments, a SARM1 inhibitor and NAD+ or a NAD+ precursor (e.g., NR, NA, NaR, NAM, NMN, NaMN, TRP, vitamin B₃, or NAAD) are co-administered to a subject. In some embodiments, a SARM1 inhibitor and one or more NAD+ precursors (e.g., NR, NA, NaR, NAM, NMN, NaMN, TRP, vitamin B₃, or NAAD) are co-administered to a subject. In some embodiments, a SARM1 inhibitor is administered to a subject exposed to NAD+ or a NAD+ precursor (e.g., NR, NA, NaR, NAM, NMN, NaMN, TRP, vitamin B₃, or NAAD). In some embodiments, a SARM1 inhibitor and NAD+ or a NAD+ precursor (e.g., NR, NA, NaR, NAM, NMN, NaMN, TRP, vitamin B₃, or NAAD) are each administered sequentially. In some embodiments, a subject is first administered a SARM1 inhibitor followed by administration of NAD+ or a NAD+ precursor (e.g., NR, NA, NaR, NAM, NMN, NaMN, TRP, vitamin B₃, or NAAD). In some embodiments, NAD+ or a NAD+ precursor (e.g., NR, NA, NaR, NAM, NMN, NaMN, TRP, vitamin B₃, or NAAD) is administered prior to the SARM1 inhibitor. In some embodiments, a SARM1 inhibitor is administered to a subject who is or has been administered NAD+ or a NAD+ precursor (e.g., NR, NA, NaR, NAM, NMN, NaMN, TRP, vitamin B₃, or NAAD).

In some embodiments, provided methods and/or combination therapies inhibit activity of SARM1. Alternatively or additionally, in some embodiments, provided methods and/or combination therapies alleviate one or more attributes of neurodegeneration. In some embodiments, the present disclosure provides methods of treating, preventing, and/or ameliorating a neurodegenerative disease, disorder or condition associated with axonal degeneration.

In some embodiments, the SARM1 inhibitor is a small molecule, a polypeptide, a peptide fragment, a nucleic acid (e.g., a siRNA, an antisense oligonucleotide, a micro-RNA, or an aptamer), an antibody, or a ribozyme.

In some embodiments, the SARM1 inhibitor is a small molecule. In some embodiments, the SARM1 inhibitor is a siRNA. In some embodiments, the SARM1 inhibitor is an antisense oligonucleotide. In some embodiments, the SARM1 inhibitor is a polypeptide. In some embodiments, a SARM1 inhibitor is a peptide fragment. In some embodiments, a SARM1 inhibitor is a nucleic acid. In some embodiments, a SARM1 inhibitor is an antisense oligonucleotide.

In some embodiments, the present disclosure provides compositions that comprise and/or deliver a SARM1 inhibitor (e.g., in a form as described herein), a prodrug or active metabolite thereof. In certain embodiments, a composition comprising a SARM1 inhibitor is formulated for use in administering to a subject in combination with NAD+ or a NAD+ precursor (e.g., NR, NA, NaR, NAM, NMN, NaMN, TRP, vitamin B₃, or NAAD).

In some embodiments, provided methods and/or combination therapies promote the increase of intracellular levels of nicotinamide adenine dinucleotide (NAD+) in cells and tissues for improving cell and tissue survival. In some embodiments, provided methods and/or combination therapies prevent a decrease in NAD+ levels in cells and/or tissues. In some embodiments, provided methods and/or combination therapies reduce NAD+ catabolism. In further embodiments, provided methods and/or combination therapies increase NAD+ levels in cells and tissues and for improving cell and tissue survival. In some embodiments, provided methods reduce or inhibit the ability of SARM1 to efficiently bind to NAD+. In some embodiments, provided combination therapies and/or methods stabilize the neurons and/or cells until the external environment stabilizes following an acute event.

In some embodiments, the present disclosure provides compositions comprising a SARM1 inhibitor for use in combination with NAD+ or a NAD+ precursor (e.g., NR, NA, NaR, NAM, NMN, NaMN, TRP, vitamin B₃, or NAAD). In some embodiments, such compositions are pharmaceutical compositions that include at least one pharmaceutically acceptable carrier, diluent or excipient. In some embodiments, the present disclosure provides compositions that comprise and/or deliver a compound including a SARM1 inhibitor with NAD+ or a NAD+ precursor (e.g., NR, NA, NaR, NAM, NMN, NaMN, TRP, vitamin B₃, or NAAD). In some embodiments, such compositions are pharmaceutically acceptable compositions that include at least one pharmaceutically acceptable carrier.

### SARM1 Inhibitors

In some embodiments, the SARM1 inhibitor is a small molecule, a polypeptide, a peptide fragment, a nucleic acid (e.g., a siRNA, an antisense oligonucleotide, a micro-RNA, or an aptamer), an antibody, or a ribozyme.

In some embodiments, the SARM1 inhibitor is a small molecule. In some embodiments, the SARM1 inhibitor is a siRNA. In some embodiments, the SARM1 inhibitor is an antisense oligonucleotide. In some embodiments, the SARM1 inhibitor is a polypeptide. In some embodiments, a SARM1 inhibitor is a peptide fragment. In some embodiments, a SARM1 inhibitor is a nucleic acid. In some embodiments, a SARM1 inhibitor is an antisense oligonucleotide.

In some embodiments, provided SARM1 inhibitors bind to SARM1 within a pocket comprising one or more catalytic residues (e.g., a catalytic cleft of SARM1). In some embodiments, provided SARM1 inhibitors inhibit SARM1 activity by binding to an allosteric site.

i. Small Molecule SARM1 Inhibitors

In some embodiments, the SARM1 inhibitor is a small molecule.

In some embodiments, the SARM1 inhibitor is selected from a compound of formula I, II, or III:

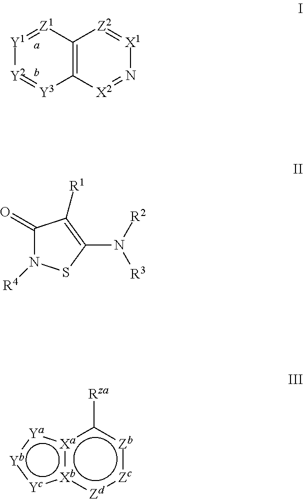

or a pharmaceutically acceptable salt thereof, wherein each of X¹, X², Y¹, Y², Y³, Z¹, Z², [symbol] , [symbol] , R¹, R², R³, R⁴, X^{a}, X^{b}, Y^{a}, Y^{b}, Y^{c}, Z^{b}, Z^{c}, Z^{d} and R^{za} is as defined, infra.

In some embodiments, the SARM1 inhibitor is a compound of formula I:

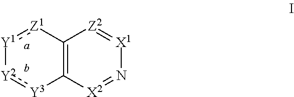

or a pharmaceutically acceptable salt thereof, wherein:
each of [symbol] and [symbol] is independently a single or double bond;
X¹ is selected from N and C—R^{x1};
R^{x1} is selected from halogen, —CN, —R′, and —OR′;
X² is selected from N and C—R^{x2};
R^{x2} is selected from halogen, —CN, —R′, —OR′, —N(R′)₂, —SO₂R′, —C(O)R′, —N(R′)SO₂R′, —SO₂N(R′)₂, —OC(O)R′, —C(O)OR′, —N(R′)C(O)R′, —C(O)N(R′)₂, and —N(R′)C(O)N(R′)₂;
Y¹ is selected from N and C—R^{y1} when [symbol] is a double bond or Y¹ is CH(R^{y1}) or C(R^{y1})₂ when [symbol] is a single bond;
R^{y1} is selected from halogen, —CN, —R′, —OR′, and —N(R)₂;
Y² is selected from N and C—R^{y2} when [symbol] is a double bond or Y² is selected from N—R′ and C(O) when [symbol] is a single bond;
Y³ is selected from N and C—R^{y3} when [symbol] is a double bond or Y³ is selected from N—R′ and C(O) when [symbol] is a single bond;
each R^{y2} and R^{y3} is independently selected from halogen, —CN, —R′, —OR′ and —N(R′)₂; and
Z¹ is selected from N and C—R^{z1} when [symbol] is a double bond or Z¹ is CH(R^{z1}) or C(R^{z1})₂ when [symbol] is a single bond;
R^{z1} is selected from halogen, —CN, —NO₂, —R′, —(C₁₋₆ alkylene)OR′, —(C₁₋₆ alkylene)N(R′)₂, —OR′, —SR′, —SF₅, —N(R′)₂, —C(O)R′, —C(O)OR′, —OC(O)R′, —C(O)N(R′)₂, —N(R′)C(O)R′, —SOR′, —SO₂R′, —N(R)SO₂R′, and —SO₂N(R′)₂;
Z² is selected from N and C—R^{z2};
R^{z2} is selected from halogen, —CN, —R′, —OR′, and —N(R′)₂; and
each R′ is independently selected from hydrogen, C₁₋₆ alkyl, C₂₋₆ alkenyl, and C₂₋₆ alkynyl, wherein each of C₁₋₆ alkyl, C₂₋₆ alkenyl, or C₂₋₆ alkynyl is optionally substituted with halogen; or:
   two instances of R′, together with the nitrogen atom to which they are attached, form a 3-to 6-membered saturated or partially unsaturated heterocyclic ring.

In some embodiments, the SARM1 inhibitor is a compound of formula I:

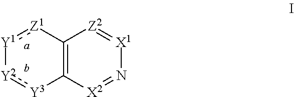

or a pharmaceutically acceptable salt thereof, wherein:
each of [symbol] and [symbol] is independently a single or double bond;
X¹ is selected from N and C—R^{x1};
R^{x2} is selected from halogen, —CN, —R′, and —OR′;
X² is selected from N and C—R^{x2};
R^{x2} is selected from halogen, —CN, —R′, —OR′, —N(R′)₂, —SO₂R′, —C(O)R′, —N(R′)SO₂R′, —SO₂N(R′)₂, —OC(O)R′, —C(O)OR′, —N(R′)C(O)R′, —C(O)N(R′)₂, and —N(R′)C(O)N(R′)₂;
Y¹ is selected from N and C—R^{y1} when [symbol] is a double bond or Y¹ is CH(R^{y1}) or C(R^{y1})₂ when [symbol] is a single bond;
R^{y1} is selected from halogen, —CN, —R′, —OR′, and —N(R′)₂;
Y² is selected from N and C—R^{y2} when [symbol] is a double bond or Y² is selected from N—R′ and C(O) when [symbol] is a single bond;
Y³ is selected from N and C—R^{y3} when [symbol] is a double bond or Y³ is selected from N-k and C(O) when [symbol] is a single bond;
each R^{y2} and R^{y3} is independently selected from halogen, —CN, —R′, —OR′ and —N(R′)₂; and
Z¹ is selected from N and C—R^{z1} when [symbol] is a double bond or Z¹ is CH(R^{z1}) or C(R^{z1})₂ when [symbol] is a single bond;
R^{z1} is selected from halogen, —CN, —NO₂, —R′, —(C₁₋₆ alkylene)OR′, —(C₁₋₆ alkylene)N(R′)₂, —OR′, —SR′, —SF₅, —N(R)₂, —C(O)R′, —C(O)OR′, —OC(O)R′, —C(O)N(k)₂, —N(R′)C(O)R′, —SOR′, —SO₂R′, —N(R′)SO₂R′, and —SO₂N(R′)₂;
Z² is selected from N and C—R^{z2};
R^{z2} is selected from halogen, —CN, —R′, —OR′, and —N(R′)₂; and
each R′ is independently selected from hydrogen, C₁₋₆ alkyl, C₂₋₆ alkenyl, and C₂₋₆ alkynyl, wherein each of C₁₋₆ alkyl, C₂₋₆ alkenyl, or C₂₋₆ alkynyl is optionally substituted with halogen; or:
   two instances of R′, together with the nitrogen atom to which they are attached, form a 3-to 6-membered saturated or partially unsaturated heterocyclic ring.

As defined generally above for formula I, each of [symbol] and [symbol] is independently a single or double bond. In some embodiments of formula I, each of [symbol] and [symbol] is a double bond. In some embodiments of formula I, each of [symbol] and [symbol] is a single bond. In some embodiments of formula I, [symbol] is a single bond and [symbol] is a double bond. In some embodiments of formula I, [symbol] is a double bond and [symbol] is a single bond.

It will be appreciated that compounds of formula I having the structure

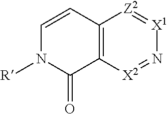

can exist in two tautomeric forms when R′ is H:

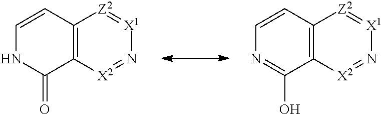

Accordingly, it will be appreciated that compounds of formula I wherein Y² is N—H and Y³ is C(O) can be drawn in either tautomeric form.

Similarly, compounds of formula I having the structure

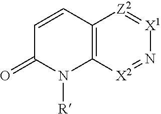

can exist in two tautomeric forms when R′ is H:

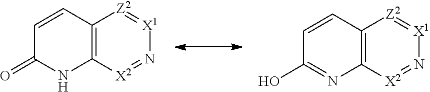

Accordingly, it will be appreciated that compounds of formula I wherein Y² is C(O) and Y³ is N—H can be drawn in either tautomeric form.

As defined generally above for formula I, X¹ is selected from N and C—R^{x1}. In some embodiments of formula I, X¹ is N. In some embodiments of formula I, X¹ is C—R^{x1}.

As defined generally above for formula I, R^{x1} is selected from halogen, —CN, —R′, and —OR′. In some embodiments of formula I, R^{x1} is —R′. In some such embodiments of formula I, R′ is H. Accordingly, in some embodiments of formula I, R^{x1} is H. In some embodiments of formula I, R^{x1} is —R′, wherein R′ is —C₁₋₆ alkyl. In some embodiments of formula I, R^{x1} is —R′, wherein R′ is —CH₃. Accordingly, in some embodiments of formula I, R^{x1} is —CH₃.

In some embodiments of formula I, R^{x1} is —OR′. In some embodiments of formula I, R^{x1} is —OR′, wherein R′ is H. Accordingly, in some embodiments of formula I, R^{x1} is —OH.

As defined generally above for formula I, X² is selected from N and C—R^{x2}. In some embodiments of formula I, X² is N. In some embodiments of formula I, X² is C—R^{x2}.

As defined generally above for formula I, R^{x2} is selected from halogen, —CN, —R′, —OR′, —N(R′)₂, —SO₂R′, —C(O)R′, —N(R′)SO₂R′, —SO₂N(R′)₂, —OC(O)R′, —C(O)OR′, —N(R′)C(O)R′, —C(O)N(R′)₂, and —N(R′)C(O)N(R′)₂. In some embodiments of formula I, R^{x2} is —R′. In some such embodiments of formula I, R′ is H. Accordingly, in some embodiments of formula I, R^{x2} is H. In some embodiments of formula I, R^{x2} is —R′, wherein R′ is —C₁₋₆ alkyl. In some embodiments of formula I, R^{x2} is —R′, wherein R′ is —CH₃. Accordingly, in some embodiments of formula I, R^{x2} is —CH₃.

In some embodiments of formula I, R^{x2} is halogen. In some embodiments of formula I, R^{x2} is chloro.

In some embodiments of formula I, R^{x2} is —N(R′)SO₂R′. In some embodiments of formula I, R^{x2} is —NHSO₂R′. In some such embodiments of formula I, R′ is —C₁₋₆ alkyl. In some embodiments of formula I, R^{x2} is —NHSO₂R′, wherein R′ is —CH₃. In some embodiments of formula I, R^{x2} is —NHSO₂R′, wherein R′ is —CH₂CH₃. In some embodiments of formula I, R^{x2} is —NHSO₂R′, wherein R′ is cyclopropyl.

In some embodiments of formula I, R^{x2} is —N(R′)₂. In some such embodiments of formula I, each R′ is H. Accordingly, in some embodiments of formula I, R^{x2} is —NH₂. In some embodiments of formula I, R^{x2} is —N(R′)₂, wherein each R′ is independently selected from H and —C₁₋₆ alkyl. In some embodiments of formula I, R^{x2} is —N(R′)₂, wherein each R′ is independently selected from H and —CH₃. In some embodiments of formula I, R^{x2} is —NHCH₃. In some embodiments, R^{x2} is —N(CH₃)₂.

In some embodiments of formula I, R^{x2} is —OR′. In some such embodiments of formula I, R′ is H. Accordingly, in some embodiments of formula I, R^{x2} is —OH. In some embodiments of formula I, R^{x2} is —OR′, wherein R′ is —C₁₋₆ alkyl. In some embodiments of formula I, R^{x2} is —OR′, wherein R′ is —CH₃. Accordingly, in some embodiments of formula I, R^{x2} is —OCH₃.

In some embodiments of formula I, R^{x2} is —N(R′)C(O)N(R′)₂. In some such embodiments of formula I, each R′ is independently selected from H and —C₁₋₆ alkyl. In some embodiments of formula I, R^{x2} is —N(R′)C(O)N(R′)₂, wherein each R′ is independently selected from H and —CH₃. In some embodiments of formula I, R^{x2} is —NHC(O)NHCH₃.

As defined generally above for formula I, Y¹ is selected from N and C—R^{y1} when [symbol] is a double bond or Y¹ is CH(R^{y1}) or C(R^{y1})₂ when [symbol] is a single bond. In some embodiments of formula I, [symbol] is a double bond and Y¹ is N. In some embodiments of formula I, [symbol] is a double bond and Y¹ is C—R^{y1}. In some embodiments of formula I, [symbol] is a single bond and Y¹ is CH(R^{y1}). In some embodiments of formula I, [symbol] is a single bond and Y¹ is C(R^{y1})₂.

As defined generally above for formula I, R is selected from halogen, —CN and —R′. In some embodiments of formula I, R^{y1} is —R′. In some such embodiments of formula I, —R′ is H. Accordingly, in some embodiments of formula I, R^{y1} is H. In some embodiments of formula I, R is —N(R′)₂. In some embodiments of formula I, R is —NH₂. In some embodiments of formula I, R^{y1} is —OR′. In some embodiments of formula I, R^{y1} is —OCH₃. In some embodiments of formula I, R^{y1} is —OH. In some embodiments of formula I, R^{y1} is halogen. In some such embodiments of formula I, R^{y1} is fluoro or bromo.

As defined generally above for formula I, Y² is selected from N and C—R^{y2} when [symbol] is a double bond or Y² is selected from N—R′ and C(O) when [symbol] is a single bond. In some embodiments of formula I, [symbol] is a double bond and Y² is N. In some embodiments of formula I, [symbol] is a double bond and Y² is C—R^{y2}. In some embodiments of formula I, [symbol] is a single bond and Y² is N—R′. In some embodiments of formula I, [symbol] is a single bond and Y² is C(O).

As defined generally above for formula I, Y³ is selected from N and C—R^{y3} when [symbol] is a double bond or Y³ is selected from N—R′ and C(O) when [symbol] is a single bond. In some embodiments of formula I, [symbol] is a double bond and Y³ is N. In some embodiments of formula I, [symbol] is a double bond and Y³ is C—R^{y3}. In some embodiments of formula I, [symbol] is a single bond and Y³ is N—R′. In some embodiments of formula I, [symbol] is a single bond and Y³ is C(O).

As defined generally above for formula I, each R^{y2} and R^{y3} is independently selected from halogen, —CN, —R′, —OR′ and —N(R′)₂. In some embodiments of formula I, R^{y2} is —R′. In some such embodiments of formula I, —R′ is H. Accordingly, in some embodiments of formula I, R^{y2} is H. In some embodiments of formula I, R^{y2} is halogen. In some such embodiments of formula I, R^{y2} is fluoro or bromo. In some embodiments of formula I, R^{y2} is —OR′. In some such embodiments of formula I, R′ is H. Accordingly, in some embodiments of formula I, R^{y2} is —OH. In some embodiments of formula I, R^{y2} is —OR′, wherein R′ is —C₁₋₆ alkyl. In some embodiments of formula I, R^{y2} is —OCH₃.

In some embodiments of formula I, R^{y3} is —R′. In some such embodiments of formula I, —R′ is H. Accordingly, in some embodiments of formula I, R^{y3} is H. In some embodiments of formula I, R^{y3} is —R′, wherein R′ is —C₁₋₆ alkyl. In some such embodiments of formula I, —R′ is CH₃. Accordingly, in some embodiments of formula I, R^{y3} is CH₃. In some embodiments of formula I, R^{y3} is halogen. In some such embodiments of formula I, R^{y3} is chloro or bromo. In some embodiments of formula I, R^{y3} is —OR′. In some such embodiments of formula I, R′ is H. Accordingly, in some embodiments of formula I, R^{y3} is —OH. In some embodiments of formula I, R^{y3} is —OR′, wherein R′ is —C₁₋₆ alkyl. In some embodiments of formula I, R^{y3} is —OCH₃.

In some embodiments of formula I, R^{y3} is —N(R′)₂. In some such embodiments of formula I, each R′ is H. Accordingly, in some embodiments of formula I, R^{y3} is —NH₂. In some embodiments of formula I, R^{y3} is —N(R′)₂, wherein each R′ is independently selected from H and —C₁₋₆ alkyl. In some such embodiments of formula I, R^{y3} is —N(R′)₂, wherein each R′ is independently selected from H and —CH₃. In some embodiments of formula I, R^{y3} is —NHCH₃. In some embodiments of formula I, R^{y3} is —N(R′)C(O)N(R′)₂. In some such embodiments of formula I, each R′ is independently selected from H and —C₁₋₆ alkyl. In some embodiments of formula I, R^{y3} is —N(R′)C(O)N(R′)₂, wherein each R′ is independently selected from H and —CH₃. In some embodiments of formula I, R^{y3} is —NHC(O)NHCH₃.

As defined generally above for formula I, Z¹ is selected from N and C—R^{z1} when [symbol] is a double bond or Z¹ is CH(R^{z1}) or C(R^{z1})₂ when [symbol] is a single bond. In some embodiments of formula I, [symbol] is a double bond and Z¹ is N. In some embodiments of formula I, [symbol] is a double bond and Z¹ is C—R^{z1}. In some embodiments of formula I, [symbol] is a single bond and Z¹ is CH(R^{z1}). In some embodiments of formula I, [symbol] is a single bond and Z¹ is C(R^{z1})₂.

As defined generally above for formula I, R^{z1} is selected from halogen, —CN, —NO₂, —R′, —(C₁₋₆ alkylene)OR′, —(C₁₋₆ alkylene)N(R′)₂, —OR′, —SR′, —SF₅, —N(R′)₂, —C(O)R′, —C(O)OR′, —OC(O)R′, —C(O)N(R′)₂, —N(R′)C(O)R′, —SOR′, —SO₂R′, —N(R′)SO₂R′, and —SO₂N(R′)₂. In some embodiments of formula I, R^{z1} is —R′. In some such embodiments of formula I, R′ is H. Accordingly, in some embodiments of formula I, R^{z1} is H.

In some embodiments of formula I, R^{z1} is halogen. In some such embodiments of formula I, R^{z1} is bromo. In some embodiments of formula I, R^{z1} is iodo. In some embodiments of formula I, R^{z1} is chloro.

In some embodiments of formula I, R^{z1} is —NO₂.

In some embodiments of formula I, R^{z1} is —CF₃.

In some embodiments of formula I, R^{z1} is —C(O)R′. In some such embodiments of formula I, R′ is —C₁₋₆ alkyl. In some embodiments of formula I, R^{z1} is —C(O)CH₃.

In some embodiments of formula I, R^{z1} is —C(O)OR′. In some such embodiments of formula I, R′ is selected from H and —C₁₋₆ alkyl. In some embodiments of formula I, R^{z1} is —C(O)OH. In some embodiments of formula I, R^{z1} is —C(O)OCH₃.

In some embodiments of formula I, R^{z1} is —N(R′)₂. In some such embodiments of formula I, each R′ is H. Accordingly, in some embodiments of formula I, R^{z1} is —NH₂.

In some embodiments of formula I, R^{z1} is —R′, wherein R′ is —C₁₋₆ alkyl. In some embodiments of formula I, R^{z1} is isopropyl. In some embodiments of formula I, R^{z1} is cyclopropyl. In some embodiments of formula I, R^{z1} is —R′, wherein R′ is —C₁₋₆ alkynyl. In some embodiments of formula I, R^{z1} is —C≡CH.

In some embodiments of formula I, R^{z1} is —OR′. In some such embodiments of formula I, R′ is H. Accordingly, in some embodiments of formula I, R^{z1} is —OH. In some embodiments of formula I, R^{z1} is —OR, wherein R′ is —C₁₋₆ alkyl. In some embodiments of formula I, R^{z1} is —OCH₃. In some embodiments of formula I, R^{z1} is —OCH(CH₃)₂.

In some embodiments of formula I, R^{z1} is —SR′, wherein R′ is —C₁₋₆ alkyl. In some embodiments of formula I, R^{z1} is —SCH₃.

In some embodiments of formula I, R^{z1} is —(C₁₋₆ alkylene)OR′. In some embodiments of formula I, R^{z1} is —CH₂OR′. In some such embodiments of formula I, R′ is H. Accordingly, in some embodiments of formula I, R^{z1} is —CH₂OH. In some embodiments of formula I, R^{z1} is —C(CH₃)₂OH.

In some embodiments of formula I, R^{z1} is —(C₁₋₆ alkylene)N(R′)₂. In some embodiments of formula I, R^{z1} is —CH₂N(R′)₂. In some such embodiments of formula I, each R′ is H. Accordingly, in some embodiments of formula I, R^{z1} is —CH₂NH₂.

As defined generally above for formula I, Z² is selected from N and C—R^{z2}. In some embodiments of formula I, Z² is N. In some embodiments of formula I, Z² is C—R^{z2}.

As defined generally above for formula I, R^{z2} is selected from halogen, —CN, —R′, —OR′, and —N(R′)₂. In some embodiments of formula I, R^{z2} is —R′. In some such embodiments of formula I, R′ is H. Accordingly, in some embodiments, R^{z2} is H. In some embodiments of formula I, R^{z2} is —R′, wherein R′ is —C₁₋₆ alkyl. In some embodiments of formula I, R^{z2} is —CH₃. In some embodiments of formula I, R^{z2} is —CH(CH₃)₂. In some embodiments of formula I, R^{z2} is cyclopropyl.

In some embodiments of formula I, R^{z2} is halogen. In some embodiments of formula I, R^{z2} is bromo. In some embodiments of formula I, R^{z2} is iodo.

In some embodiments of formula I, R^{z2} is —OR′. In some such embodiments of formula I, R′ is H. Accordingly, in some embodiments of formula I, R^{z2} is —OH. In some embodiments of formula I, R^{z2} is —OR′, wherein R′ is —C₁₋₆ alkyl. Accordingly, in some embodiments of formula I, R^{z2} is —OCH₃.

In some embodiments, R^{z2} is —N(R′)₂. In some such embodiments, each R′ is H. Accordingly, in some embodiments, R^{z2} is —NH₂.

As defined generally above for formula I, each R′ is independently selected from hydrogen, C₁₋₆ alkyl, C₂₋₆ alkenyl, and C₂₋₆ alkynyl, wherein each of C₁₋₆ alkyl, C₂₋₆ alkenyl, or C₂₋₆ alkynyl is optionally substituted with halogen; or two instances of R′, together with the nitrogen atom to which they are attached, form a 3- to 6-membered saturated or partially unsaturated heterocyclic ring.

In some embodiments of formula I, Z¹ is C—R^{z1} and Z² is C—R^{z2}. Accordingly, the present disclosure provides a compound of formula I-a:

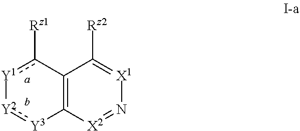

or a pharmaceutically acceptable salt thereof.

In some embodiments of formula I, Z¹ is C—R^{z1}, Z² is C—R^{z2}, and each of [symbol] and [symbol] is a double bond. Accordingly, in some embodiments of formula I, the SARM1 inhibitor is a compound of formula I-b:

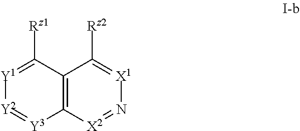

or a pharmaceutically acceptable salt thereof.

In some embodiments of formula I, [symbol] is a double bond, [symbol] is a single bond, Y² is N—R′, and Y³ is C(O). Accordingly, in some embodiments of formula I, the SARM1 inhibitor is a compound of formula I-c:

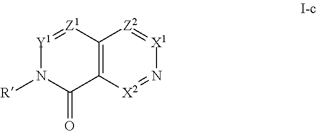

or a pharmaceutically acceptable salt thereof.

In some embodiments of formula I of formula I [symbol] is a single bond, Y² is N—R′, and Y³ is C(O). Accordingly, in some embodiments of formula I, the present disclosure provides a compound of formula I-d:

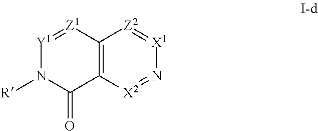

or a pharmaceutically acceptable salt thereof.

In some embodiments of formula I, [symbol] is a double bond, Y² is C(O), and Y³ is N—R′. Accordingly, in some embodiments of formula I, the present disclosure provides a compound of formula I-e:

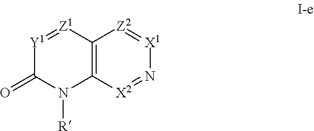

or a pharmaceutically acceptable salt thereof.

In some embodiments of formula I, [symbol] is a single bond, Y² is C(O), and Y³ is N—R′. Accordingly, in some embodiments of formula I, the present disclosure provides a compound of formula I-f:

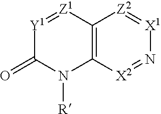

or a pharmaceutically acceptable salt thereof.

In some embodiments of formula I, X² is C—R^{x2}, Y¹ is C—R^{y1}, Y² is C—R^{y2}, and Y³ is C—R^{y3}. Accordingly, in some embodiments of formula I, the present disclosure provides a compound of formula I-g:

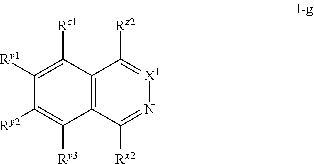

or a pharmaceutically acceptable salt thereof.

In some embodiments of formula I, R^{x2} is H. Accordingly, in some embodiments of formula I, the present disclosure provides a compound of formula I-h:

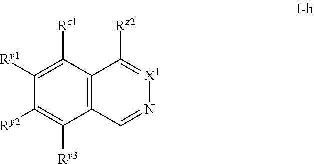

or a pharmaceutically acceptable salt thereof.

In some embodiments of formula I, R^{y1} is H. Accordingly, in some embodiments of formula I, the present disclosure provides a compound of formula I-i:

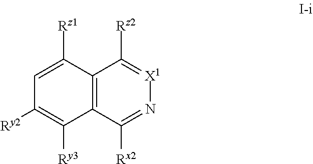

or a pharmaceutically acceptable salt thereof.

In some embodiments of formula I, R^{y2} is H. Accordingly, in some embodiments of formula I, the present disclosure provides a compound of formula I-j:

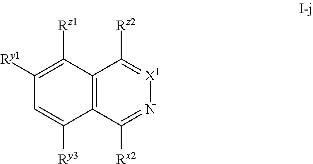

or a pharmaceutically acceptable salt thereof.

In some embodiments of formula I, R^{x1} is H. Accordingly, in some embodiments of formula I, the present disclosure provides a compound of formula I-k:

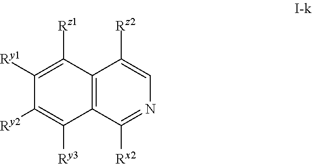

or a pharmaceutically acceptable salt thereof.

In some embodiments of formula I, the present disclosure provides a compound of any one of formula I-b-i, I-b-ii, I-b-iii, I-b-iv, I-b-v, I-b-vi, I-b-vii, I-b-viii, I-b-ix, I-b-x, I-b-xi, I-b-xii, I-b-xiii, I-b-xiv, I-b-xv, I-b-xvi, and I-b-xvii:

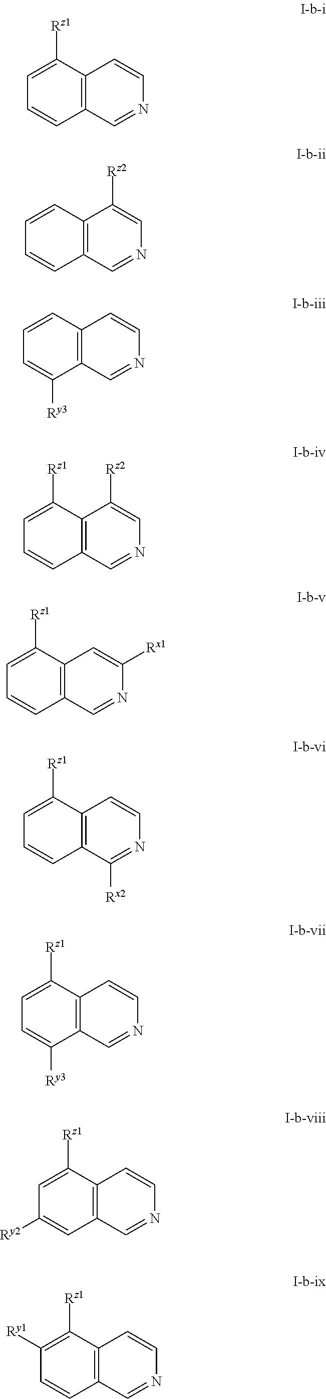

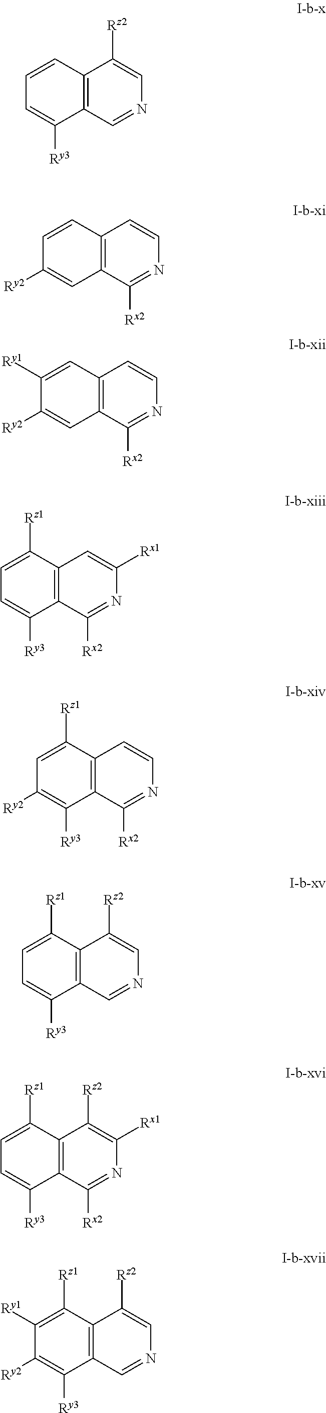

or a pharmaceutically acceptable salt thereof, wherein each of R^{x1}, R^{x2}, R^{y1}, R^{y2}, R^{y3}, R^{z1} and R^{z2} is as defined above for formula I and described herein.

In some embodiments of formula I, the present disclosure provides a compound of any one of formula I-b-xviii, I-b-xix, I-b-xx, I-b-xxi, I-b-xxii, I-b-xxiii, I-b-xxiv, I-b-xxv, and I-b-xxvi:

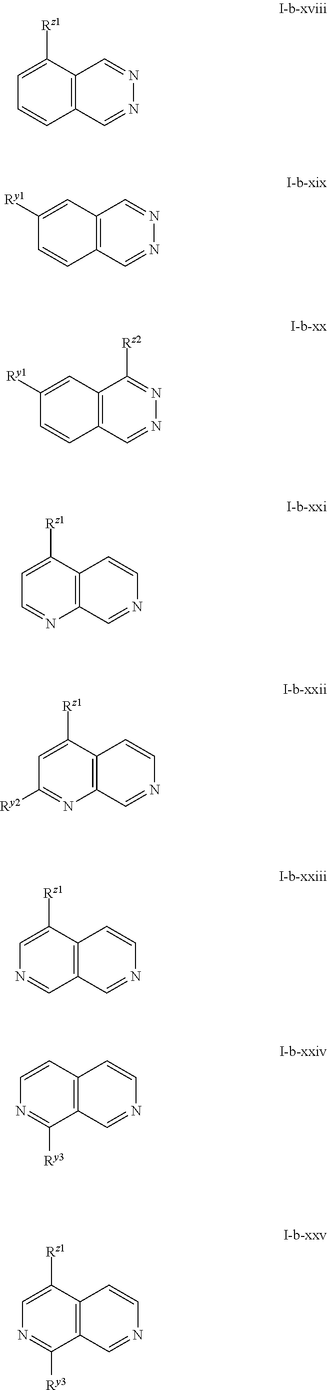

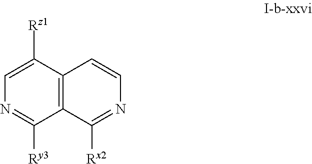

or a pharmaceutically acceptable salt thereof, wherein each of R^{x1}, R^{x2}, R^{y1}, R^{y2}, R^{y3}, R^{z1} and R^{z2} is as defined above for formula I and described herein.

In some embodiments of formula I, the present disclosure provides a compound of any one of formula I-a-i, I-a-ii, and I-a-iii, or a pharmaceutically acceptable salt thereof:

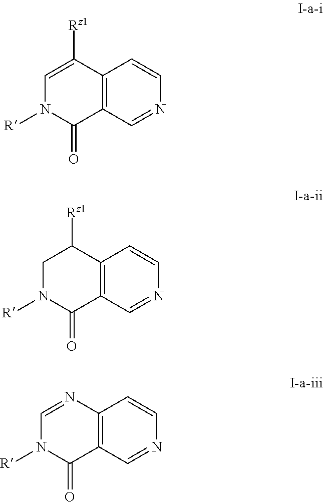

wherein each of R^{z1} and R′ is as defined above and described herein.

In some embodiments, a compound of formula I is selected from: Example Structure

| Example | Structure |
|---|---|
| I-1 | 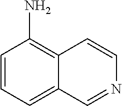 [SMILES: Nc1cccc2cnccc12] |
| I-2 | 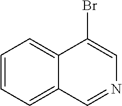 [SMILES: Brc1cncc2ccccc12] |
| I-3 | 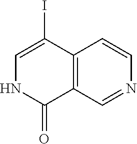 [SMILES: O=c1[nH]cc(I)c2ccncc12] |
| I-4 | 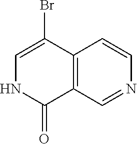 [SMILES: O=c1[nH]cc(Br)c2ccncc12] |
| I-5 | 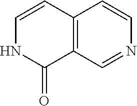 [SMILES: O=c1[nH]ccc2ccncc12] |
| I-6 | 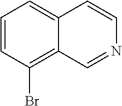 [SMILES: Brc1cccc2ccncc12] |
| I-7 | 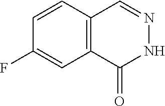 [SMILES: O=c1[nH]ncc2ccc(F)cc12] |
| I-8 | 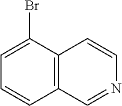 [SMILES: Brc1cccc2cnccc12] |
| I-9 | 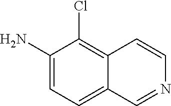 [SMILES: Nc1ccc2cnccc2c1Cl] |
| I-10 | 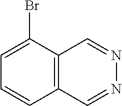 [SMILES: Brc1cccc2cnncc12] |
| I-11 | 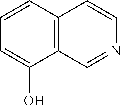 [SMILES: Oc1cccc2ccncc12] |
| I-12 | 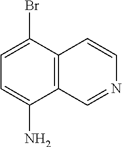 [SMILES: Nc1ccc(Br)c2ccncc12] |
| I-13 | 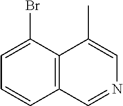 |
| I-14 | 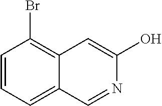 [SMILES: Oc1cc2c(Br)cccc2cn1] |
| I-15 | 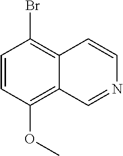 |
| I-16 | 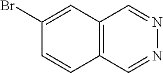 [SMILES: Brc1ccc2cnncc2c1] |
| I-17 | 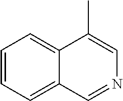 |
| I-18 | 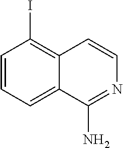 [SMILES: Nc1nccc2c(I)cccc12] |
| I-19 | 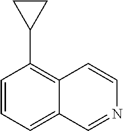 [SMILES: c1cc(C2CC2)c2ccncc2c1] |
| I-20 | 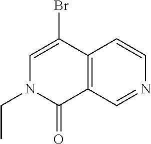 |
| I-21 | 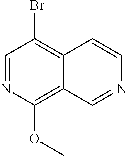 |
| I-22 | 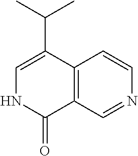 |
| I-23 | 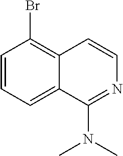 |
| I-24 | 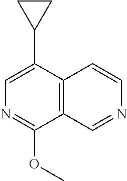 |
| I-25 | 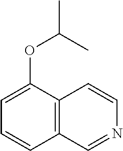 |
| I-26 | 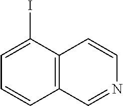 [SMILES: Ic1cccc2cnccc12] |
| I-27 | 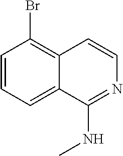 |
| I-28 | 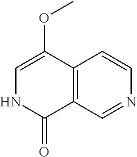 |
| I-29 | 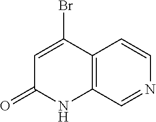 [SMILES: O=c1cc(Br)c2ccncc2[nH]1] |
| I-30 | 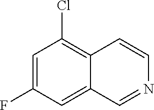 [SMILES: Fc1cc(Cl)c2ccncc2c1] |
| I-31 | 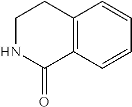 [SMILES: O=C1NCCc2ccccc21] |
| I-32 | 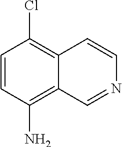 [SMILES: Nc1ccc(Cl)c2ccncc12] |
| I-33 | 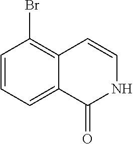 [SMILES: O=c1[nH]ccc2c(Br)cccc12] |
| I-34 | 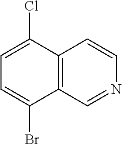 [SMILES: Clc1ccc(Br)c2cnccc12] |
| I-35 | 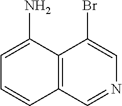 [SMILES: Nc1cccc2cncc(Br)c12] |
| I-36 | 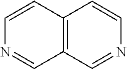 |
| I-37 | 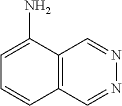 [SMILES: Nc1cccc2cnncc12] |
| I-38 | 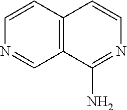 [SMILES: Nc1nccc2ccncc12] |
| I-39 | 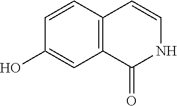 [SMILES: O=c1[nH]ccc2ccc(O)cc12] |
| I-40 | 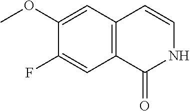 |
| I-41 | 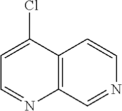 [SMILES: Clc1ccnc2cnccc12] |
| I-42 | 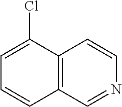 [SMILES: Clc1cccc2cnccc12] |
| I-43 | 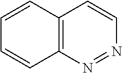 |
| I-44 | 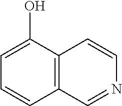 [SMILES: Oc1cccc2cnccc12] |
| I-45 | 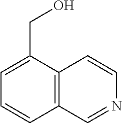 [SMILES: OCc1cccc2cnccc12] |
| I-46 | 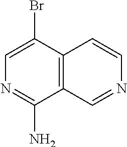 [SMILES: Nc1ncc(Br)c2ccncc12] |
| I-47 | 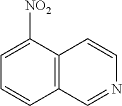 [SMILES: O=[N+]([O-])c1cccc2cnccc12] |
| I-48 | 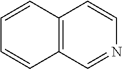 |
| I-49 | 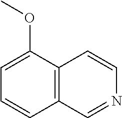 |
| I-50 | 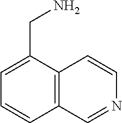 [SMILES: NCc1cccc2cnccc12] |
| I-51 | 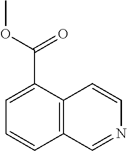 |
| I-52 | 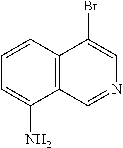 [SMILES: Nc1cccc2c(Br)cncc12] |
| I-53 | 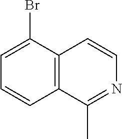 |
| I-54 | 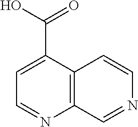 [SMILES: O=C(O)c1ccnc2cnccc12] |
| I-55 | 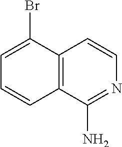 [SMILES: Nc1nccc2c(Br)cccc12] |
| I-56 | 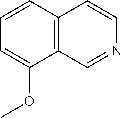 |
| I-57 | 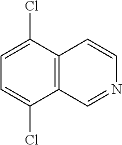 [SMILES: Clc1ccc(Cl)c2cnccc12] |
| I-58 | 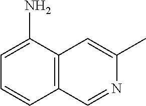 |
| I-59 | 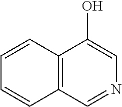 [SMILES: Oc1cncc2ccccc12] |
| I-60 | 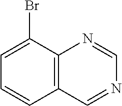 [SMILES: Brc1cccc2cncnc12] |
| I-61 | 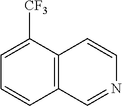 [SMILES: FC(F)(F)c1cccc2cnccc12] |
| I-62 | 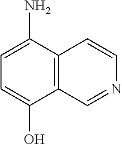 [SMILES: Nc1ccc(O)c2cnccc12] |
| I-63 | 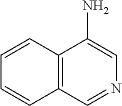 [SMILES: Nc1cncc2ccccc12] |
| I-64 | 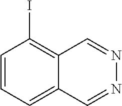 [SMILES: Ic1cccc2cnncc12] |
| I-65 | 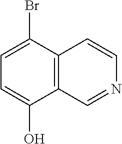 [SMILES: Oc1ccc(Br)c2ccncc12] |
| I-66 | 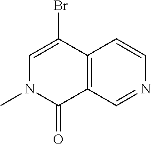 |
| I-67 | 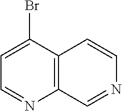 [SMILES: Brc1ccnc2cnccc12] |
| I-68 | 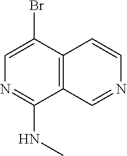 |
| I-69 | 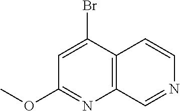 |
| I-70 | 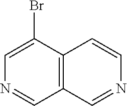 [SMILES: Brc1cncc2cnccc12] |
| I-71 | 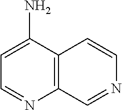 [SMILES: Nc1ccnc2cnccc12] |
| I-72 | 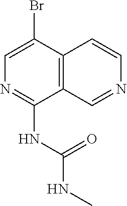 [SMILES: CNC(=O)Nc1ncc(Br)c2ccncc12] |
| I-73 | 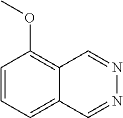 |
| I-74 | 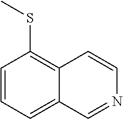 |
| I-75 | 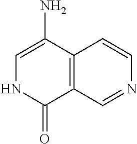 [SMILES: Nc1c[nH]c(=O)c2cnccc12] |
| I-76 | 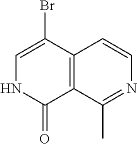 |
| I-77 | 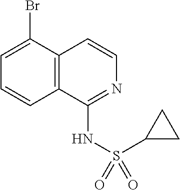 [SMILES: O=S(=O)(Nc1nccc2c(Br)cccc12)C1CC1] |
| I-78 | 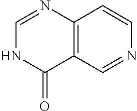 [SMILES: O=c1[nH]cnc2ccncc12] |
| I-79 | 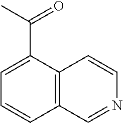 |
| I-80 | 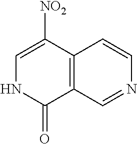 [SMILES: O=c1[nH]cc([N+](=O)[O-])c2ccncc12] |
| I-81 | 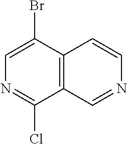 [SMILES: Clc1ncc(Br)c2ccncc12] |
| I-82 | 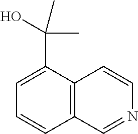 |
| I-83 | 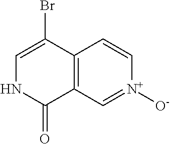 [SMILES: O=c1[nH]cc(Br)c2cc[n+]([O-])cc12] |
| I-84 | 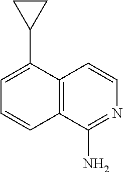 [SMILES: Nc1nccc2c(C3CC3)cccc12] |
| I-85 | 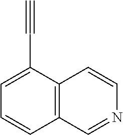 |
| I-86 | 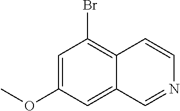 |

or a pharmaceutically acceptable salt thereof.

In some embodiments, the SARM1 inhibitor is a compound of formula II:

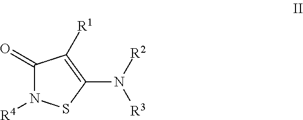

or a pharmaceutically acceptable salt thereof, wherein
R¹ is selected from —CN, —NO₂, —C(O)R″, —S(O)₂R″, —CON(R″)₂, —S(O)₂N(R″)₂, and —CO₂R″;
R² is —R″;
R³ is —(CH₂)₀₋₂Cy, or:
   R² and R³, together with the nitrogen atom to which they are attached, form a 4- to 7-membered saturated or partially unsaturated ring fused to Cy or a 4- to 7-membered saturated or partially unsaturated ring substituted with -Cy;
Cy is selected from phenyl, a 5- to 6-membered heteroaryl ring having 1-3 heteroatoms independently selected from nitrogen, oxygen, and sulfur, an 8- to 10-membered bicyclic heteroaryl ring having 1-3 heteroatoms independently selected from nitrogen, oxygen, and sulfur, and an 8- to 10-membered bicyclic aryl ring, wherein each phenyl, heteroaryl and aryl ring is substituted with 0-4 R^{x};
each R^{x} is independently selected from halogen, —CN, —NO₂, —OR″, —SR″, —N(R″)₂, —SO₂R″, —SO₂N(R″)₂, —CO₂R″, —CON(R″)₂, —N(R″)SO₂R″, —N(R″)C(O)R″, and optionally substituted C₁₋₆ aliphatic;
R⁴ is —R″;
each R″ is independently hydrogen or optionally substituted C₁₋₆ aliphatic, or:
   two instances of R″, together with the atom to which they are attached, form a 3- to 6-membered saturated or partially unsaturated heterocyclic ring.

As defined generally above for formula II, R¹ is selected from —CN, —NO₂, —C(O)R″, —S(O)₂R″, —CON(R″)₂, —S(O)₂N(R″)₂, and —CO₂R″. In some embodiments of formula II, R¹ is selected from —CN, —C(O)N(R″)₂ and —CO₂R″. In some embodiments of formula II, R¹ is —CN. In some embodiments, R¹ is —CON(R″)₂. In some such embodiments of formula II, each R″ is independently selected from hydrogen and C₁₋₆ aliphatic. In some embodiments of formula II, R¹ is —CON(R″)₂, wherein each R is independently selected from hydrogen and C₁₋₆ alkyl. In some embodiments of formula II, R¹ is —CON(R″)₂, wherein each R″ is independently selected from hydrogen and —CH₃. In some embodiments of formula II, R¹ is —CONH₂. In some embodiments of formula II, R¹ is —CO₂R″. In some such embodiments of formula II, R″ is selected from hydrogen and C₁₋₆ aliphatic. In some embodiments of formula II, R¹ is —CO₂R″, wherein R″ is selected from hydrogen and C₁₋₆ alkyl. In some embodiments of formula II, R¹ is —CO₂R″, wherein R″ is selected from hydrogen and —CH₃. In some embodiments of formula II, R¹ is —CO₂H. In some embodiments, R¹ is —NO₂. In some embodiments of formula II, R¹ is —C(O)R″. In some embodiments of formula II, R¹ is —S(O)₂R″. In some embodiments of formula II, R¹ is —S(O)₂N(R″)₂.

As defined generally above for formula II, R² is —R″. In some such embodiments of formula II, —R″ is hydrogen. Accordingly, in some embodiments of formula II, R² is —H. In some embodiments of formula II, R² is —R″, wherein —R″ is optionally substituted C₁₋₆ aliphatic. In some embodiments of formula II, R² is —R″, wherein —R″ is C₁₋₆ aliphatic. In some embodiments of formula II, R² is —C₁₋₆ alkyl. In some such embodiments of formula II, R² is —CH₃.

As defined generally above for formula II, R³ is —(CH₂)₀₋₂Cy. In some embodiments of formula II, R³ is -Cy. In some embodiments of formula II, R³ is —CH₂-Cy. In some embodiments of formula II, R³ is —(CH₂)₂-Cy.

As defined generally above for formula II, Cy is selected from phenyl, a 5- to 6-membered heteroaryl ring having 1-3 heteroatoms independently selected from nitrogen, oxygen, and sulfur, an 8- to 10-membered bicyclic heteroaryl ring having 1-3 heteroatoms independently selected from nitrogen, oxygen, and sulfur, and an 8- to 10-membered bicyclic aryl ring, wherein each phenyl, heteroaryl and aryl ring is substituted with 0-4 R^{x}.

In some embodiments of formula II, Cy is phenyl. In some embodiments of formula II, Cy is phenyl substituted with 1 R^{x}. In some embodiments of formula II, Cy is phenyl substituted with 2 R^{x}. In some embodiments of formula II, Cy is selected from

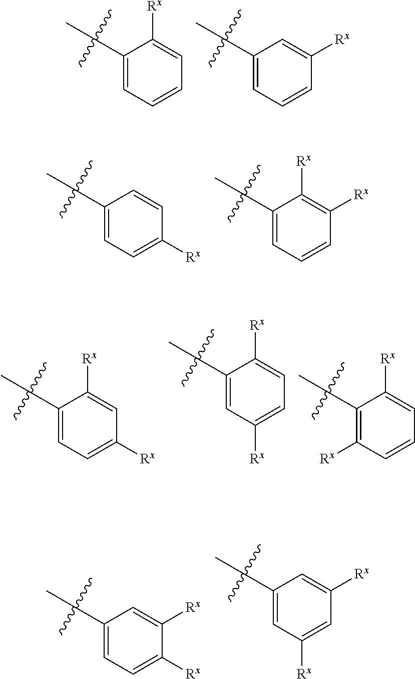

In some embodiments of formula II, Cy is a 5- to 6-membered heteroaryl ring having 1-3 heteroatoms independently selected from nitrogen, oxygen, and sulfur. In some embodiments of formula II, Cy is a 5-membered heteroaryl ring having 1-3 heteroatoms independently selected from nitrogen, oxygen, and sulfur. In some embodiments of formula II, Cy is a 6-membered heteroaryl ring having 1-3 nitrogen atoms. In some embodiments of formula II, Cy is a 6-membered heteroaryl ring having 1-2 nitrogen atoms. In some such embodiments of formula II, Cy is substituted with 1 R^{x}. In some embodiments of formula II, Cy is pyridinyl. In some such embodiments of formula II, Cy is pyrimidin-2-yl, pyrimidin-3-yl, or pyrimidin-4-yl. In some embodiments of formula II, Cy is pyridazinyl. In some embodiments of formula II, Cy is pyrazinyl. In some embodiments of formula II, Cy is pyrimidinyl. In some embodiments of formula II, Cy is selected from:

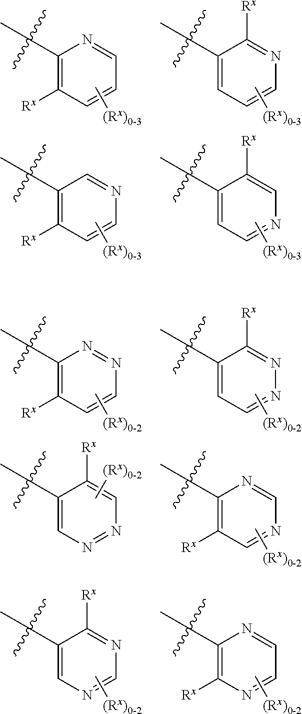

In some embodiments of formula II, Cy is an 8- to 10-membered bicyclic heteroaryl ring having 1-3 heteroatoms independently selected from nitrogen, oxygen, and sulfur. In some embodiments of formula II, Cy is an 8- to 10-membered bicyclic heteroaryl ring having 1-3 nitrogen atoms. In some embodiments of formula II, Cy is an 10-membered bicyclic heteroaryl ring having 1-3 nitrogen atoms. In some embodiments of formula II, Cy is an 10-membered bicyclic heteroaryl ring having 1 nitrogen atom. In some such embodiments of formula II, Cy is substituted with 1 R^{x}. In some embodiments of formula II, Cy is quinolin-2-yl, quinolin-3-yl, quinolin-4-yl, quinolin-5-yl, quinolin-6-yl, quinolin-7-yl, or quinolin-8-yl.

In some embodiments of formula II, Cy is an 8- to 10-membered bicyclic aryl ring. In some embodiments of formula II, Cy is a 10-membered bicyclic aryl ring. In some such embodiments of formula II, Cy is substituted with 1 R^{x}. In some embodiments, Cy is naphth-1-yl. In some embodiments of formula II, Cy is naphth-2-yl.

In some embodiments of formula II, R² and R³, together with the nitrogen atom to which they are attached, form a 4- to 7-membered saturated or partially unsaturated ring fused to Cy or a 4- to 7-membered saturated or partially unsaturated ring substituted with -Cy. In some embodiments of formula II, R² and R³, together with the nitrogen atom to which they are attached, form a ring selected from:

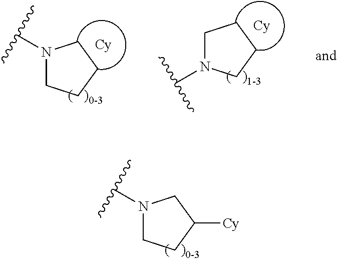

wherein Cy is substituted with 0-4 R^{x}.

As defined generally above for formula II, each R^{x} is independently selected from halogen, —CN, —NO₂, —OR″, —SR″, —N(R″)₂, —SO₂R″, —SO₂N(R″)₂, —CO₂R″, —CON(R″)₂, —N(R)SO₂R″, and —N(R″)C(O)R″, or optionally substituted C₁₋₆ aliphatic.

In some embodiments of formula II, R^{x} is halogen. In some such embodiments of formula II, R^{x} is fluoro. In some embodiments of formula II, R^{x} is chloro.

In some embodiments of formula II, R^{x} is optionally substituted C₁₋₆ aliphatic. In some embodiments of formula II, R^{x} is optionally substituted —C₁₋₆ alkyl. In some embodiments of formula II, R^{x} is —C₁₋₆ alkyl optionally substituted with halogen. In some embodiments of formula II, R^{x} is optionally substituted —CH₃. In some such embodiments of formula II, R^{x} is —CF₃.

In some embodiments of formula II, R^{x} is C₁₋₆ aliphatic. In some embodiments of formula II, R^{x} is —C₁₋₆ alkyl. In some embodiments of formula II, R^{x} is —CH₃. In some embodiments of formula II, R^{x} is —CH(CH₃)₂.

In some embodiments of formula II, R^{x} is —OR″. In some such embodiments of formula II, R″ is C₁₋₆ aliphatic. In some embodiments of formula II, R^{x} is —OR″, wherein R″ is C₁₋₆ alkyl. In some embodiments of formula II, R^{x} is —OCH₃.

In some embodiments of formula II, R^{x} is —OR. In some such embodiments of formula II, R″ is optionally substituted C₁₋₆ aliphatic. In some embodiments of formula II, R^{x} is —OR″, wherein R″ is optionally substituted C₁₋₆ alkyl. In some embodiments of formula II, R^{x} is —OR″, wherein R″ is optionally substituted —CH₃. In some embodiments of formula II, R^{x} is —OR″, wherein R″ is —CF₃. Accordingly, in some embodiments of formula II, R^{x} is —OCF₃.

In some embodiments of formula II, R^{x} is —SO₂R″. In some such embodiments of formula II, R″ is optionally substituted C₁₋₆ aliphatic. In some embodiments of formula II, R^{x} is —SO₂R″, wherein R″ is C₁₋₆ alkyl. In some embodiments of formula II, R^{x} is —SO₂R″, wherein R″ is —CH₃. Accordingly, in some embodiments of formula II, R^{x} is —SO₂CH₃.

In some embodiments of formula II, R^{x} is —SR″. In some such embodiments of formula II, R″ is optionally substituted C₁₋₆ aliphatic. In some embodiments of formula II, R^{x} is —SR″, wherein R″ is C₁₋₆ alkyl. In some embodiments of formula II, R^{x} is —SR″, wherein R″ is —CH₃. Accordingly, in some embodiments of formula II, R^{x} is —SCH₃.

As defined generally above for formula II, R⁴ is —R. In some embodiments of formula II, R⁴ is —R. In some such embodiments of formula II, —R″ is hydrogen. Accordingly, in some embodiments of formula II, R⁴ is hydrogen. In some embodiments of formula II, R⁴ is —R″, wherein R″ is optionally substituted C₁₋₆ aliphatic. In some embodiments of formula II, R⁴ is —R″, wherein R″ is C₁₋₆ aliphatic. In some embodiments of formula II, R⁴ is —R″, wherein R″ is C₁₋₆ alkyl. In some embodiments, R⁴ is —R″, wherein R″ is CH₃. Accordingly, in some embodiments of formula II, R⁴ is —CH₃.

As defined generally above for formula II, each R″ is independently hydrogen or optionally substituted C₁₋₆ aliphatic; or two instances of R″, together with the atom to which they are attached, form a 3- to 6-membered saturated or partially unsaturated heterocyclic ring.

In some embodiments of formula II, R″ is hydrogen. In some embodiments of formula II, R″ is optionally substituted C₁₋₆ aliphatic. In some such embodiments of formula II, R″ is —C₁₋₆ alkyl. In some embodiments, R″ is —CH₃.

It will be appreciated that compounds of formula II having the structure

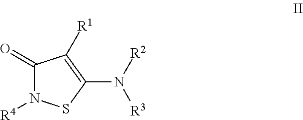

can exist in two tautomeric forms when R⁴ is H:

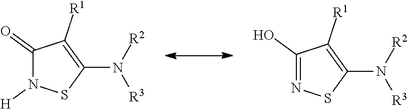

Accordingly, it will be appreciated that compounds of formula II wherein R⁴ is H can be drawn in either tautomeric form.

In some embodiments of formula II, R¹ is —CN. Accordingly, in some embodiments of formula II, the SARM1 inhibitor is a compound of formula II-a:

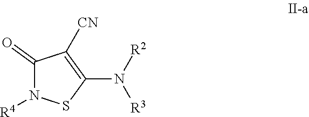

or a pharmaceutically acceptable salt thereof, wherein each of R², R³ and R⁴ is as defined above and described herein.

In some embodiments of formula II, Ri is —CON(R″)2. Accordingly, in some embodiments of formula II, the SARM1 inhibitor is a compound of formula II-b:

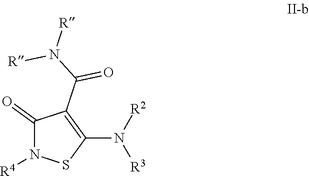

or a pharmaceutically acceptable salt thereof, wherein each of R², R³, R⁴ and R″ is as defined above and described herein.

In some embodiments of formula II-a or II-b, R² is H. Accordingly, in some embodiments, the SARM1 inhibitor is a compound of formula II-a-i or II-a-ii:

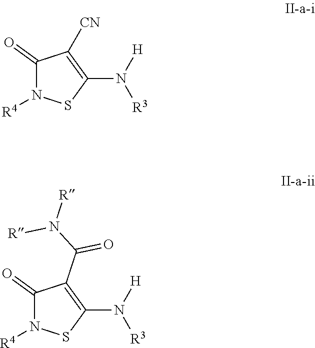

or a pharmaceutically acceptable salt thereof, wherein each of R³, R⁴ and R″ is as defined above and described herein.

In some embodiments of formula II-a or II-b, R³ is -Cy, wherein -Cy is phenyl. Accordingly, in some embodiments, the SARM1 inhibitor is a compound of formula II-b-i or II-b-ii:

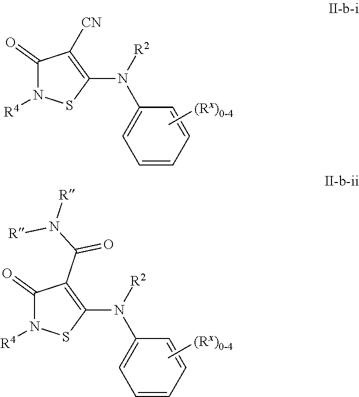

or a pharmaceutically acceptable salt thereof, wherein each of R², R⁴, R″ and R^{x} is as defined above and described herein.

In some embodiments, the compound of formula II is selected from:

| Example | Structure |
|---|---|
| II-1 | 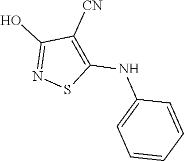 [SMILES: N#Cc1c(O)nsc1Nc1ccccc1] |
| II-2 | 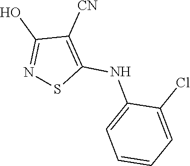 [SMILES: N#Cc1c(O)nsc1Nc1ccccc1Cl] |
| II-3 | 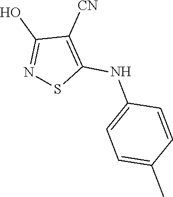 [SMILES: Cc1ccc(Nc2snc(O)c2C#N)cc1] |
| II-4 | 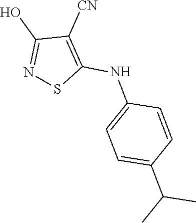 [SMILES: CC(C)c1ccc(Nc2snc(O)c2C#N)cc1] |
| II-5 | 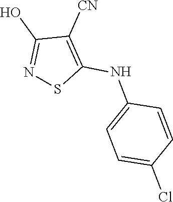 [SMILES: N#Cc1c(O)nsc1Nc1ccc(Cl)cc1] |
| II-6 | 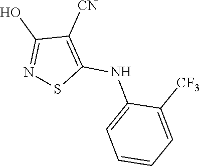 [SMILES: N#Cc1c(O)nsc1Nc1ccccc1C(F)(F)F] |
| II-7 | 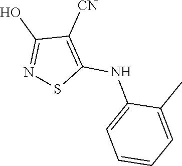 [SMILES: Cc1ccccc1Nc1snc(O)c1C#N] |
| II-8 | 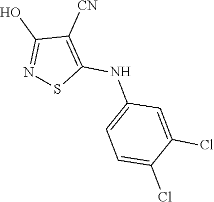 [SMILES: N#Cc1c(O)nsc1Nc1ccc(Cl)c(Cl)c1] |
| II-9 | 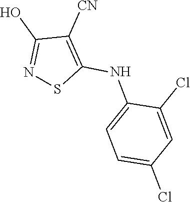 [SMILES: N#Cc1c(O)nsc1Nc1ccc(Cl)cc1Cl] |
| II-10 | 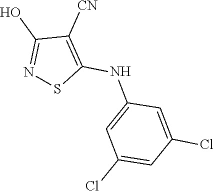 [SMILES: N#Cc1c(O)nsc1Nc1cc(Cl)cc(Cl)c1] |
| II-11 | 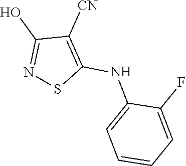 [SMILES: N#Cc1c(O)nsc1Nc1ccccc1F] |
| II-12 | 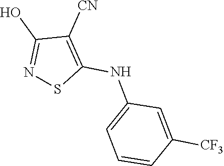 [SMILES: N#Cc1c(O)nsc1Nc1cccc(C(F)(F)F)c1] |
| II-13 | 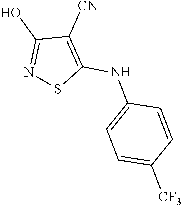 [SMILES: N#Cc1c(O)nsc1Nc1ccc(C(F)(F)F)cc1] |
| II-14 | 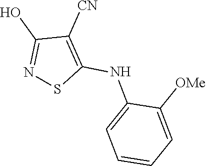 [SMILES: COc1ccccc1Nc1snc(O)c1C#N] |
| II-15 | 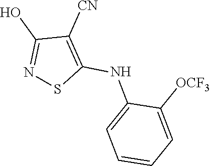 [SMILES: N#Cc1c(O)nsc1Nc1ccccc1OC(F)(F)F] |
| II-16 | 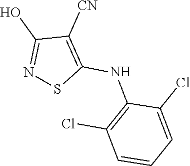 [SMILES: N#Cc1c(O)nsc1Nc1c(Cl)cccc1Cl] |
| II-17 | 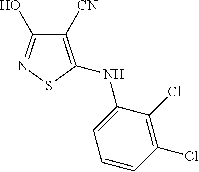 [SMILES: N#Cc1c(O)nsc1Nc1cccc(Cl)c1Cl] |
| II-18 | 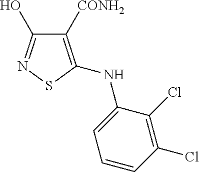 [SMILES: NC(=O)c1c(O)nsc1Nc1cccc(Cl)c1Cl] |
| II-19 | 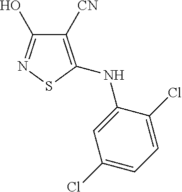 [SMILES: N#Cc1c(O)nsc1Nc1cc(Cl)ccc1Cl] |
| II-20 | 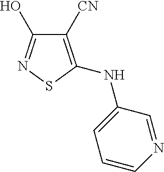 [SMILES: N#Cc1c(O)nsc1Nc1cccnc1] |
| II-21 | 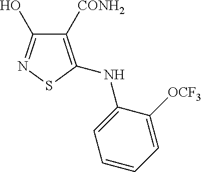 [SMILES: NC(=O)c1c(O)nsc1Nc1ccccc1OC(F)(F)F] |
| II-22 | 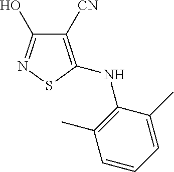 [SMILES: Cc1cccc(C)c1Nc1snc(O)c1C#N] |
| II-23 | 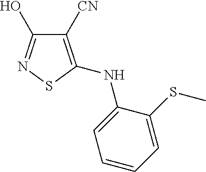 [SMILES: CSc1ccccc1Nc1snc(O)c1C#N] |
| II-24 | 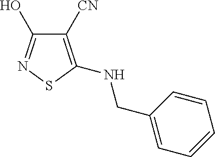 [SMILES: N#Cc1c(O)nsc1NCc1ccccc1] |
| II-25 | 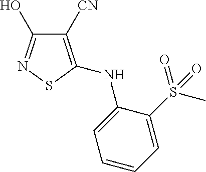 [SMILES: CS(=O)(=O)c1ccccc1Nc1snc(O)c1C#N] |
| II-26 | 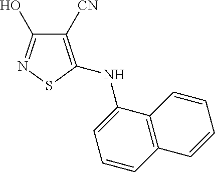 [SMILES: N#Cc1c(O)nsc1Nc1cccc2ccccc12] |
| II-27 | 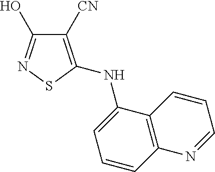 [SMILES: N#Cc1c(O)nsc1Nc1cccc2ncccc12] |
| II-28 | 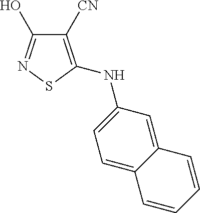 [SMILES: N#Cc1c(O)nsc1Nc1ccc2ccccc2c1] |
| II-29 | 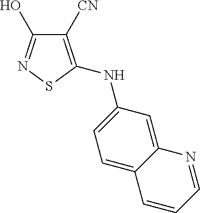 [SMILES: N#Cc1c(O)nsc1Nc1ccc2cccnc2c1] |
| II-30 | 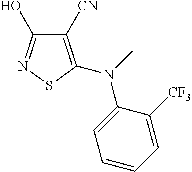 [SMILES: CN(c1ccccc1C(F)(F)F)c1snc(O)c1C#N] |
| II-31 | 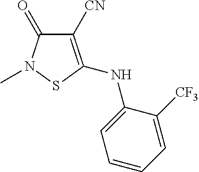 [SMILES: Cn1sc(Nc2ccccc2C(F)(F)F)c(C#N)c1=O] |
| II-32 | 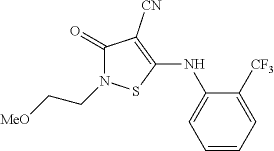 [SMILES: COCCn1sc(Nc2ccccc2C(F)(F)F)c(C#N)c1=O] |

In some embodiments, one or more compounds of formula II covalently inhibit SARM1. In some embodiments, one or more compounds of formula II covalently modify a cysteine residue of SARM1. In some embodiments, one or more compounds of formula II covalently modify Cys635 of SARM1. In some embodiments, one or more compounds of formula II covalently modify Cys629 of SARM1. In some embodiments, one or more compounds of formula II covalently modify Cys649 of SARM1.

In some embodiments, the SARM1 inhibitor is a compound of formula III:

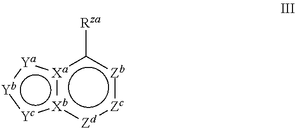

or a pharmaceutically acceptable salt thereof, wherein:
   one of X^{a} and X^{b} is selected from C and N and the other is C;
   Y^{a} is selected from N, N—R^{†} and C—R^{ya};
   Y^{b} is selected from N and C—R^{yb}
   Y^{c} is selected from N, N—R^{†}, O, S, and S(O)₂;
   Z^{b} is selected from N and C—R^{zb};
   Z^{c} is selected from N and C—R^{zc};
   Z^{d} is selected from N and C—R^{zd}.
   each R^{†} is independently selected from hydrogen and C₁₋₆ aliphatic optionally substituted with —OR′″, —C(O)N(R′″)₂, or —C(O)OR′″;
   each of R^{ya}, R^{yb}, R^{za}, R^{zb}, R^{zc}, and R^{zd} is independently selected from hydrogen, halogen, —CN, —OR′″, —C(O)OR′″, and C₁₋₆ aliphatic optionally substituted with halogen, —CN, —OR′″, —N(R′″)₂, —C(O)OR′″, or —C(O)N(R′″)₂; and
   each R′″ is independently selected from hydrogen and C₁₋₆ aliphatic;
      or two instances of R′″, together with the atom to which they are attached, form a 3- to 6-membered saturated or partially unsaturated heterocyclic ring.

As defined generally above for formula III, one of X^{a} and X^{b} is selected from C and N and the other is C. In some embodiments of formula III, X^{a} is N and X^{b} is C. In some embodiments of formula III, X^{a} is C and X^{b} is N.

It will be appreciated that compounds of formula III wherein one of X^{a} and X^{b} is N have the structures:

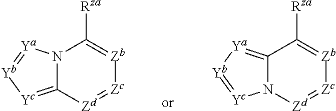

It is therefore understood that, due to the valence of Y^{a} and Y^{c} in such compounds of formula III, (i) Y^{a} is selected from N and C—R^{y} and (ii) Y^{c} is N.

As defined generally above for formula III, each R^{†} is independently selected from hydrogen and C₁₋₆ aliphatic optionally substituted with —OR′″, —C(O)N(R′″)₂, or —C(O)OR″. In some embodiments of formula III, R^{†} is hydrogen. In some embodiments of formula III, R^{†} is C₁₋₆ aliphatic optionally substituted with —OR′″, —C(O)N(R′″)₂, or —C(O)OR′″. In some embodiments of formula III, R^{†} is C₁₋₆ aliphatic. In some such embodiments of formula III, R^{†} is C₁₋₆ alkyl. In some embodiments of formula III, R^{†} is —CH₃. In some embodiments of formula III, R^{†} is —CH₂CH₃. In some embodiments of formula III, R^{†} is —CH(CH₃)₂.

In some embodiments of formula III, R^{†} is C₁₋₆ aliphatic optionally substituted with —OR′″. In some embodiments of formula III, R^{†} is C₁₋₆ alkylene optionally substituted with —OR′″. In some embodiments of formula III, R^{†} is C₁₋₄ alkylene optionally substituted with —OR′″. In some embodiments of formula III, R^{†} is C₁₋₃ alkylene optionally substituted with —OR′″. In some embodiments of formula III, R^{†} is C₁₋₂ alkylene optionally substituted with —OR′″. In some embodiments, R^{†} is —(CH₂)₁₋₃OR′″. In some embodiments of formula III, R^{†} is —(CH₂)₂₋₃OR′″. In some embodiments of formula III, R^{†} is —(CH₂)₂OR′″. In some embodiments of formula III, R^{†} is —(CH₂)₃OR′″.

In some embodiments of formula III, R^{†} is C₁₋₆ aliphatic optionally substituted with —C(O)OR′″. In some embodiments of formula III, R^{†} is C₁₋₆ alkylene optionally substituted with —C(O)OR′″. In some embodiments of formula III, R^{†} is C₁₋₄ alkylene optionally substituted with —C(O)OR′″. In some embodiments of formula III, R^{†} is C₁₋₃ alkylene optionally substituted with —C(O)OR′″. In some embodiments of formula III, R^{†} is C₁₋₂ alkylene optionally substituted with —C(O)OR′″. In some embodiments of formula III, R^{†} is —(CH₂)₁₋₃C(O)OR′″. In some embodiments of formula III, R^{†} is —(CH₂)₂₋₃C(O)OR′″. In some embodiments of formula III, R^{†} is —CH₂C(O)OR′″. In some embodiments of formula III, R^{†} is —(CH₂)₂C(O)OR′″.

In some embodiments of formula III, R^{†} is C₁₋₆ aliphatic optionally substituted with —C(O)N(R)₂. In some embodiments of formula III, R^{†} is C₁₋₆ alkylene optionally substituted with —C(O)N(R′″)₂. In some embodiments of formula III, R^{†} is C₁₋₄ alkylene optionally substituted with —C(O)N(R′″)₂. In some embodiments of formula III, R^{†} is C₁₋₃ alkylene optionally substituted with —C(O)N(R′″)₂. In some embodiments of formula III, R^{†} is C₁₋₂ alkylene optionally substituted with —C(O)N(R′″)₂. In some embodiments of formula III, R^{†} is —(CH₂)₁₋₃C(O)N(R′″)₂. In some embodiments of formula III, R^{†} is —(CH₂)₂₋₃C(O)N(R′″)₂. In some embodiments of formula III, R^{†} is —CH₂C(O)N(R′″)₂. In some embodiments of formula III, R^{†} is —(CH₂)₂C(O)N(R′″)₂.

As defined generally above for formula III, each of R^{ya}, R^{yb}, R^{za}, R^{zb}, R^{zc}, and R^{zd} is independently selected from hydrogen, halogen, —CN, —OR′″, —C(O)OR′″, and C₁₋₆ aliphatic optionally substituted with halogen, —CN, —OR′″, —N(R′″)₂, —C(O)OR′″, or —C(O)N(R′″)₂. In some embodiments of formula III, R^{ya} is hydrogen. In some embodiments of formula III, R^{ya} is halogen, —CN, —OR′″, —C(O)OR′″, or C₁₋₆ aliphatic optionally substituted with halogen, —CN, —OR′″, —N(R′″)₂, —C(O)OR′″, or —C(O)N(R′″)₂. In some embodiments of formula III, R^{ya} is hydrogen, halogen or —OR′″. In some embodiments of formula III, R^{ya} is halogen. In some such embodiments of formula III, R^{ya} is chloro. In some embodiments of formula III, R^{ya} is bromo. In some embodiments of formula III, R^{ya} is iodo. In some embodiments of formula III, R^{ya} is —OR′″. In some embodiments of formula III, R^{ya} is —CN or —C(O)OR″.

In some embodiments of formula III, R^{yb} is hydrogen. In some embodiments of formula III, R^{yb} is halogen, —CN, —OR′″, —C(O)OR′″, or C₁₋₆ aliphatic optionally substituted with halogen, —CN, —OR′″, —N(R′″)₂, —C(O)OR′″, or —C(O)N(R′″)₂. In some embodiments of formula III, R^{yb} is hydrogen, —CN, —C(O)OR′″ or C₁₋₆ aliphatic. In some embodiments of formula III, R^{yb} is C₁₋₆ aliphatic. In some such embodiments of formula III, R^{yb} is C₁₋₆ alkyl. In some embodiments of formula III, R^{yb} is —CH₃. In some embodiments of formula III, R^{yb} is —CN. In some embodiments of formula III, R^{yb} is —C(O)OR′″. In some embodiments of formula III, R^{yb} is —OR″.

In some embodiments of formula III, R^{za} is hydrogen. In some embodiments of formula III, R^{za} is halogen, —CN, —OR′″, —C(O)OR′″, or C₁₋₆ aliphatic optionally substituted with halogen, —CN, —OR′″, —N(R′″)₂, —C(O)OR′″, or —C(O)N(R′″)₂. In some embodiments of formula III, R^{za} is hydrogen or halogen. In some embodiments of formula III, R^{za} is halogen. In some such embodiments of formula III, R^{za} is bromo. In some embodiments of formula III, R^{za} is —OR. In some embodiments of formula III, R^{za} is —CN or —C(O)OR′″.

In some embodiments of formula III, R^{zb} is hydrogen. In some embodiments of formula III, R^{zb} is halogen, —CN, —OR′″, —C(O)OR′″, or C₁₋₆ aliphatic optionally substituted with halogen, —CN, —OR′″, —N(R′″)₂, —C(O)OR′″, or —C(O)N(R′″)₂. In some embodiments of formula III, R^{zb} is C₁₋₆ aliphatic optionally substituted with halogen, —CN, —OR′″, —N(R′″)₂, —C(O)OR′″, or —C(O)N(R′″)₂. In some embodiments of formula III, R^{zb} is hydrogen or C₁₋₆ aliphatic. In some embodiments of formula III, R^{zb} is C₁₋₆ aliphatic. In some such embodiments of formula III, R^{zb} is C₁₋₆ alkyl. In some embodiments of formula III, R^{zb} is —CH₃. In some embodiments of formula III, R^{zb} is —OR. In some embodiments of formula III, R^{zb} is —CN or —C(O)OR′″.

In some embodiments of formula III, R^{zc} is hydrogen. In some embodiments of formula III, R^{zc} is halogen, —CN, —OR′″, —C(O)OR′″, or C₁₋₆ aliphatic optionally substituted with halogen, —CN, —OR′″, —N(R′″)₂, —C(O)OR′″, or —C(O)N(R′″)₂. In some embodiments of formula III, R^{zc} is —OR′″. In some embodiments of formula III, R^{zc} is —CN or —C(O)OR′″. In some embodiments of formula III, R^{zc} is C₁₋₆ aliphatic optionally substituted with halogen, —CN, —OR′″, —N(R′″)₂, —C(O)OR′″, or —C(O)N(R′″)₂.

In some embodiments of formula III, R^{zd} is hydrogen. In some embodiments of formula III, R^{zd} is halogen, —CN, —OR′″, —C(O)OR′″, or C₁₋₆ aliphatic optionally substituted with halogen, —CN, —OR′″, —N(R′″)₂, —C(O)OR′″, or —C(O)N(R′″)₂. In some embodiments of formula III, R^{zd} is halogen. In some such embodiments of formula III, R^{zd} is chloro. In some embodiments of formula III, R^{zd} is —OR. In some embodiments of formula III, R^{zd} is C₁₋₆ aliphatic optionally substituted with halogen, —CN, —OR′″, —N(R′″)₂, —C(O)OR′″, or —C(O)N(R′″)₂. In some embodiments of formula III, R^{zd} is —C(O)OR′″. In some embodiments of formula III, R^{zd} is —CN.

As defined generally above for formula III, Y^{a} is selected from N, N—R^{†} and C—R^{ya}. In some embodiments of formula III, Y^{a} is N. In some embodiments of formula III, Y^{a} is N—R^{†}. In some embodiments of formula III, Y^{a} is C—R^{ya}.

As defined generally above for formula III, Y^{b} is selected from N and C—R^{yb}. In some embodiments of formula III, Y^{b} is N. In some embodiments of formula III, Y^{b} is C—R^{Y}.

As defined generally above for formula III, Y^{c} is selected from N, N-Rt, O, S, and S(O)₂. In some embodiments of formula III, Y^{c} is selected from N—R^{†}, O, S, and S(O)₂. In some embodiments of formula III, Y^{c} is selected from N—R^{†}, O, and S. In some embodiments of formula III, Y^{c} is N. In some embodiments of formula III, Y^{c} is N—R^{†}. In some embodiments of formula III, Y^{c} is O. In some embodiments of formula III, Y^{c} is S. In some embodiments of formula III, Y^{c} is S(O)₂.

As defined generally above for formula III, Z^{b} is selected from N and C—R^{zb}. In some embodiments of formula III, Z^{b} is N. In some embodiments of formula III, Z^{b} is C—R^{zb}.

As defined generally above for formula III, Z^{c} is selected from N and C—R^{zc}. In some embodiments of formula III, Z^{c} is N. In some embodiments of formula III, Z^{c} is C—R^{zc}.

As defined generally above for formula III, Z^{d} is selected from N and C—R^{zd}. In some embodiments of formula III, Z^{d} is N. In some embodiments of formula III, Z^{d} is C—R^{zd}.

As defined generally above for formula III, each R′″ is independently selected from hydrogen and C₁₋₆ aliphatic, or two instances of R′″, together with the atom to which they are attached, form a 3- to 6-membered saturated or partially unsaturated heterocyclic ring. In some embodiments of formula III, R′″ is hydrogen. In some embodiments of formula III, R′″ is C₁₋₆ aliphatic. In some such embodiments of formula III, R′″ is C₁₋₆ alkyl. In some embodiments of formula III, R′″ is —CH₃. In some embodiments of formula III, R′″ is selected from hydrogen and —CH₃.

In some embodiments of formula III, Z^{c} is N. Accordingly, in some embodiments, the SARM1 inhibitor is a compound of formula III-a:

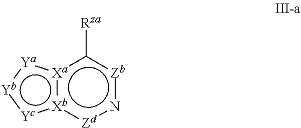

or a pharmaceutically acceptable salt thereof.

In some embodiments of formula III, X^{a} is N and X^{b} is C. Accordingly, in some embodiments, the SARM1 inhibitor is a compound of formula III-b:

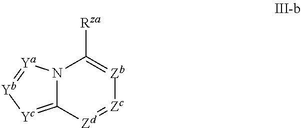

or a pharmaceutically acceptable salt thereof.

In some embodiments of Formula III, X^{a} is C and X^{b} is N. Accordingly, in some embodiments, the SARM1 inhibitor is a compound of formula III-c:

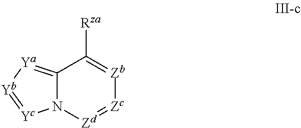

or a pharmaceutically acceptable salt thereof.

In some embodiments of formula III, the SARM1 inhibitor is a compound of any one of formula III-a-i, III-a-ii, III-a-iii, III-a-iv, III-a-v, III-b-i, III-b-ii, III-c-i, or III-c-ii:

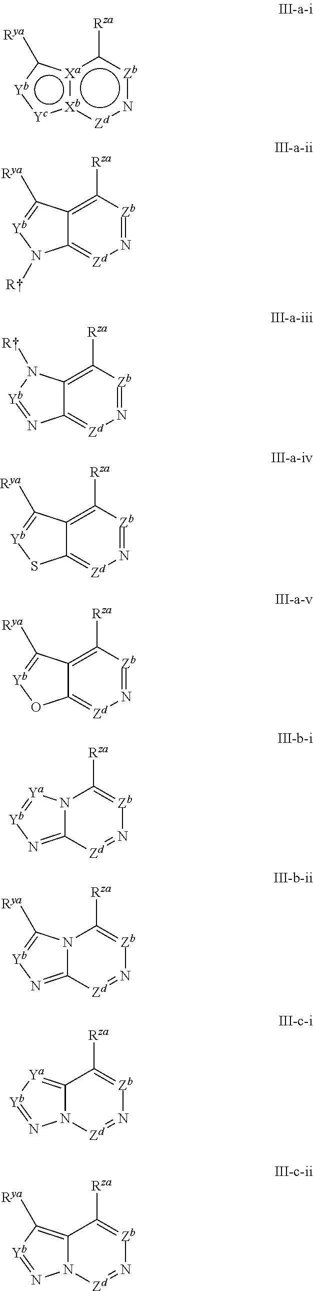

or a pharmaceutically acceptable salt thereof, wherein each of X^{a}, X^{b}, Y^{a}, Y^{b}, Y^{c}, Z^{b}, Z^{d}, R^{ya}, R^{za}, and R^{†} is as defined above and described herein.

In some embodiments, a compound of formula III is selected from:

| Example | Structure |
|---|---|
| III-1 | 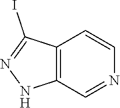 |
| III-2 | 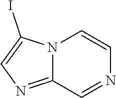 |
| III-3 | 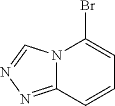 |
| III-4 | 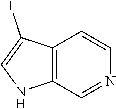 |
| III-5 | 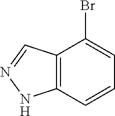 |
| III-6 | 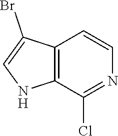 [SMILES: Clc1nccc2c(Br)c[nH]c12] |
| III-7 | 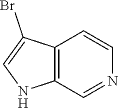 |
| III-8 | 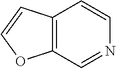 |
| III-9 | 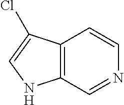 |
| III-10 | 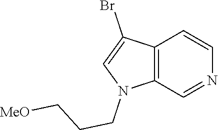 [SMILES: COCCCn1cc(Br)c2ccncc21] |
| III-11 | 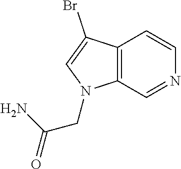 [SMILES: NC(=O)Cn1cc(Br)c2ccncc21] |
| III-12 | 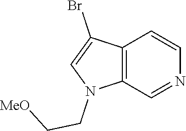 [SMILES: COCCn1cc(Br)c2ccncc21] |
| III-13 | 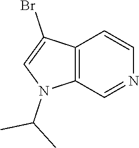 |
| III-14 | 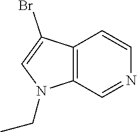 |
| III-15 | 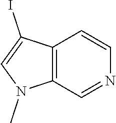 |
| III-16 | 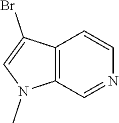 |
| III-17 | 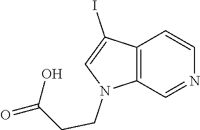 [SMILES: O=C(O)CCn1cc(I)c2ccncc21] |
| III-18 | 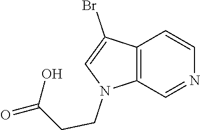 [SMILES: O=C(O)CCn1cc(Br)c2ccncc21] |
| III-19 | 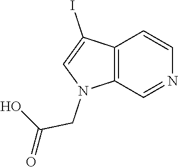 [SMILES: O=C(O)Cn1cc(I)c2ccncc21] |
| III-20 | 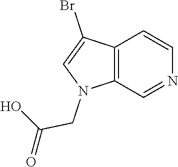 [SMILES: O=C(O)Cn1cc(Br)c2ccncc21] |
| III-21 | 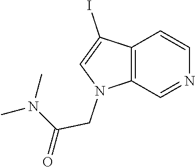 [SMILES: CN(C)C(=O)Cn1cc(I)c2ccncc21] |
| III-22 | 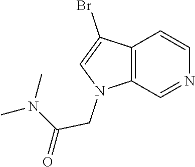 [SMILES: CN(C)C(=O)Cn1cc(Br)c2ccncc21] |
| III-23 | 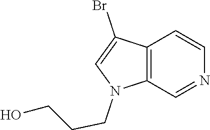 [SMILES: OCCCn1cc(Br)c2ccncc21] |
| III-24 | 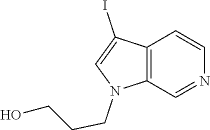 [SMILES: OCCCn1cc(I)c2ccncc21] |
| III-25 | 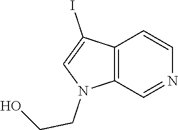 [SMILES: OCCn1cc(I)c2ccncc21] |
| III-26 | 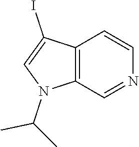 |
| III-27 | 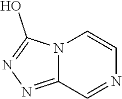 |
| III-28 | 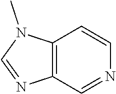 |
| III-29 | 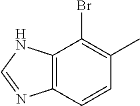 |
| III-30 | 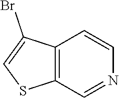 |
| III-31 | 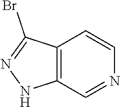 |
| III-32 | 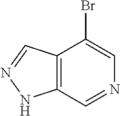 |
| III-33 | 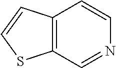 |
| III-34 | 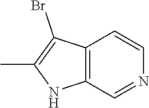 |
| III-35 | 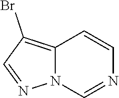 |
| III-36 | 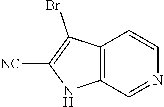 [SMILES: N#Cc1[nH]c2cnccc2c1Br] |
| III-37 | 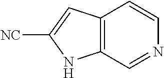 [SMILES: N#Cc1cc2ccncc2[nH]1] |
| III-38 | 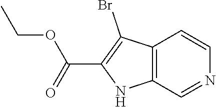 |
| III-39 | 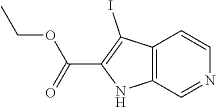 |
| III-40 | 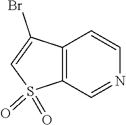 [SMILES: O=S1(=O)C=C(Br)c2ccncc21] |
| III-41 | 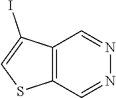 |
| III-42 | 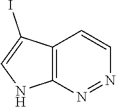 |
| III-43 | 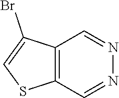 |
| III-44 | 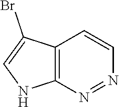 |

or a pharmaceutically acceptable salt thereof.

### Compositions

In some embodiments, the present disclosure provides compositions that comprise and/or deliver a SARM1 inhibitor (e.g., in a form as described herein), a prodrug or active metabolite thereof. In certain embodiments, a composition comprising a SARM1 inhibitor is formulated for use in administering to a subject in combination with NAD+ or a NAD+ precursor (e.g., NR, NA, NaR, NAM, NMN, NaMN, TRP, vitamin B₃, or NAAD). In some embodiments, the present disclosure provides compositions comprising a SARM1 inhibitor for use in combination with NAD+ or a NAD+ precursor (e.g., NR, NA, NaR, NAM, NMN, NaMN, TRP, vitamin B₃, or NAAD). In some embodiments, such compositions are pharmaceutical compositions that include at least one pharmaceutically acceptable carrier, diluent or excipient. In some embodiments, the present disclosure provides compositions that comprise and/or deliver a compound of Formula I, II, or III with NAD+ or a NAD+ precursor (e.g., NR, NA, NaR, NAM, NMN, NaMN, TRP, vitamin B₃, or NAAD). In some embodiments, such compositions are pharmaceutically acceptable compositions that include at least one pharmaceutically acceptable carrier.

In some embodiments, provided methods comprise administering a composition comprising a SARM1 inhibitor and one or more pharmaceutically acceptable excipients.

The amount of SARM1 inhibitor in provided compositions is such that is effective to measurably inhibit axonal degeneration and/or measurably affect a change in a biomarker of neurodegeneration in a biological sample or in a subject. In certain embodiments, a composition comprising a SARM1 inhibitor is formulated for administration to a subject in need of such composition. The compounds and compositions, according to the methods of the present disclosure, may be administered using any amount and any route of administration effective for treating or lessening the severity of any disease or disorder described herein. SARM1 inhibitors are preferably formulated in unit dosage form for ease of administration and uniformity of dosage. The expression “unit dosage form” as used herein refers to a physically discrete unit of agent appropriate for the subject to be treated. It will be understood, however, that the total daily usage of the SARM1 inhibitors will be decided by the attending physician within the scope of sound medical judgment. The specific effective dose level for any particular subject or organism will vary from subject to subject, depending on a variety of factors, including the disorder being treated and the severity of the disorder; the activity of the specific compound employed; the specific composition employed and its route of administration; the species, age, body weight, sex and diet of the subject; the general condition of the subject; the time of administration; the rate of excretion of the specific compound employed; the duration of the treatment; drugs used in combination or coincidental with the specific compound employed, and the like.

### Exemplification

The present teachings including descriptions provided in the Examples that are not intended to limit the scope of any claim. Unless specifically presented in the past tense, inclusion in the Examples is not intended to imply that the experiments were actually performed. The following non-limiting examples are provided to further illustrate the present teachings. Those of skill in the art, in light of the present disclosure, will appreciate that many changes can be made in the specific embodiments that are disclosed and still obtain a like or similar result without departing from the spirit and scope of the present teachings.

### Example 1

Activated SARM1 is a highly effective NADase that depletes local axonal NAD+ reserves within minutes to a few hours after activation, leading to a local bioenergetic crisis within this important neuronal compartment, followed by rapid axonal degeneration. The axon degeneration assay, as described herein, demonstrates the effect of treating injured axons with a SARM1 inhibitor in combination with NR.

### Materials and Methods

Methods and compositions described herein utilize laboratory techniques well known to persons skilled in the art, and can be found in laboratory manuals such as Sambrook, J., et al., Molecular Cloning: A Laboratory Manual, 3rd ed. Cold Spring Harbor Laboratory Press, Cold Spring Harbor, N.Y., 2001; Methods In Molecular Biology, ed. Richard, Humana Press, NJ, 1995; Spector, D. L. et al., Cells: A Laboratory Manual, Cold Spring Harbor Laboratory Press, Cold Spring Harbor, N.Y., 1998; and Harlow, E., Using Antibodies: A Laboratory Manual, Cold Spring Harbor Laboratory Press, Cold Spring Harbor, N.Y., 1999. Methods of administration of pharmaceuticals and dosage regimes, can be determined according to standard principles of pharmacology, using methods provided by standard reference texts such as Remington: the Science and Practice of Pharmacy (Alfonso R. Gennaro ed. 19th ed. 1995); Hardman, J. G., et al., Goodman & Gilman's The Pharmacological Basis of Therapeutics, Ninth Edition, McGraw-Hill, 1996; and Rowe, R. C., et al., Handbook of Pharmaceutical Excipients, Fourth Edition, Pharmaceutical Press, 2003.

### Mouse DRG Drop Culture

Mouse dorsal root ganglion neurons (DRGs) were dissected out of E12.5 CD1 mice (50 ganglia per embryo) and incubated with 0.5% Trypsin solution containing 0.02% EDTA (Gibco) at 37° C. for 15 min. The cells were then triturated by gentle pipetting and washed 3 times with DRG growth medium (Neurobasal medium (Gibco) containing 2% B27 (Invitrogen), 100 ng/ml 2.5S NGF (Harland Bioproducts), 1 mM 5-fluoro-2′deoxyuridine (Sigma), penicillin, and streptomycin). Cells were suspended in the DRG growth medium. DRG drop cultures were created by spotting 5000 cells/well into the center of each well of a 96-well tissue culture plate coated with poly-D-Lysine (0.1 mg/ml; Sigma) and laminin (3 mg/ml; Invitrogen). Cells were allowed to adhere to the plates in a humidified tissue culture incubator (5% CO₂) for 15 min and then DRG growth medium was gently added (100 ml well).

### Axon Degeneration Assay

To study the axonal protective effects of combining NR supplementation with a SARM1 inhibitor, 6 day-old mouse DRG drop cultures were preincubated with 100 μM NR for 24 hours before axotomy. 2 hours prior to axotomy, DRG cultures were treated with SARM1 inhibitors, in the continued presence of 100 μM NR. Potent SARM1 inhibitors were selected from two classes: isoquinoline and isothiazole SARM1 inhibitors. Isoquinoline SARM1 inhibitors tested included I-26 and I-86, while isothiazole SARM1 inhibitors tested included II-6 and II-32. The SARM1 inhibitors were tested using concentrations ranging from 0.1 to 33 μM.

A manual axotomy was performed at time 0 by transecting the axons of the DRG neurons with a blade. After axotomy, DRG cultures remained exposed to the to SARM1 inhibitor alone, 100 μM NR alone, or the combination of SARM1 inhibitor and NR. At either 16 or 24 hours, DRG cultures were fixed in a buffered solution containing 1% PFA and sucrose and stored at 4° C. prior to imaging. Bright-field images of DRG axons and cell bodies were collected using the 20× water immersion lens of a Phenix automated confocal microscope (PerkinElmer) and quantitation of axonal damage was performed using in-house developed scripts (Acapella, PerkinElmer). The effect of NR alone in protecting distal axons from fragmentation was determined at a concentration of 100 μM. The effect of combining 100 μM NR with varying concentrations of a SARM1 inhibitor was compared to the individual protective effects of either 100 μM of NR alone or an equivalent concentration of a SARM1 inhibitor alone.

### Results

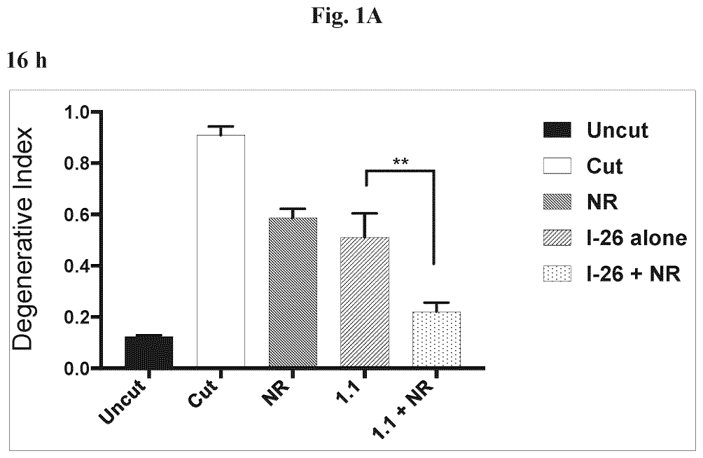

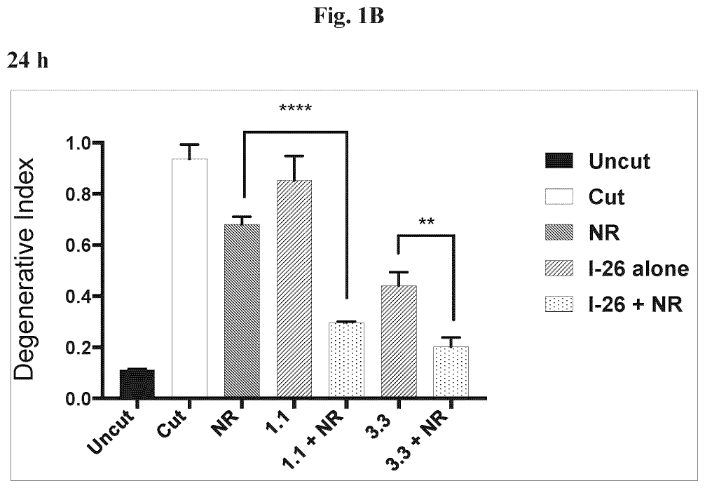

A potent SARM1 inhibitor, I-26, was used to assess the axonal protection conferred when applied in combination with NR on the axon degeneration assay described herein. As shown in FIGS. 1A and 1B, the combination of compound I-26+NR extends neuroprotection post-axotomy as compared to single agent therapy. FIGS. 1A and 1B show the degeneration index of DRG axons at 16 and 24 hours post-axotomy, respectively. For each concentration of compound I-26 tested, the extent of axonal protection of a combination of compound I-26+NR was always compared to the amount of protection produced by the agent in that combination that, individually, had the greater protective effect. In FIG. 1A, at 16 h, I-26 or NR alone provides a modest amount of axonal protection, which is similar for both agents. The combination of I-26+NR provided a statistically significant and substantially greater protection than either I-26 or NR alone. In FIG. 1B, at 24 h, NR alone provided a modest level of protection, whereas 1.1 μM of compound I-26 alone afforded no statistically significant benefit. Surprisingly, the combination of 1.1 μM compound I-26+NR provided robust and statistically significant protection. Furthermore, the magnitude of the combined effect of compound I-26 and NR is greater than the sum of the individual effects of either agent alone, indicating that the effect of combining this agent is not simply additive but in fact synergistic and could not have been predicted from the individual effect of each agent in isolation. At the higher 3.3 μM dose of compound I-26, axons show more protection than with NR alone. Furthermore, the combination of 3.3 μM compound I-26+NR showed a statistically significant benefit than with compound I-26 alone.

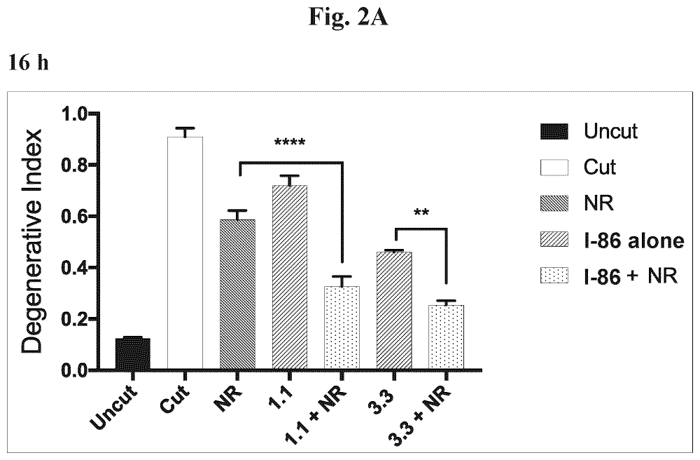

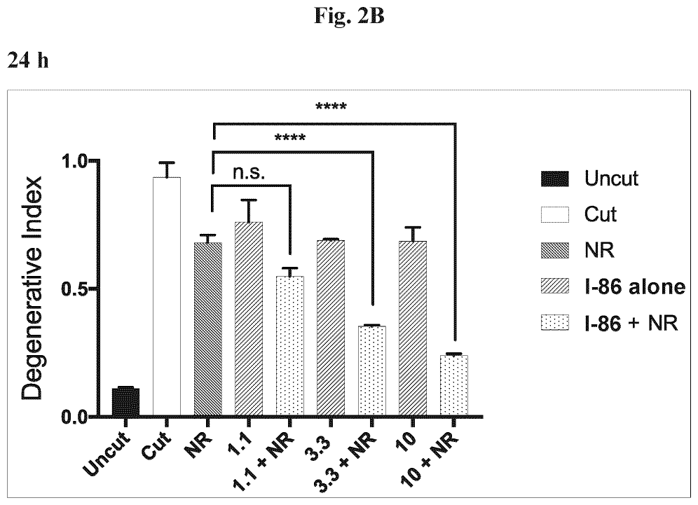

A potent SARM1 inhibitor, I-86, was used to assess the axonal protection conferred when applied in combination with NR on the axon degeneration assay described herein. FIGS. 2A and 2B show the degeneration index of DRG axons at 16 and 24 hours post-axotomy, respectively. For each concentration of compound I-86 tested, the extent of axonal protection of a combination of compound I-86+NR was always compared to the amount of protection produced by the agent in that combination that, individually, had the greater protective effect. In FIG. 2A, at 16 h, NR alone provided greater protection than 1.1 μM compound I-86 alone, whereas 3.3 μM compound I-86 alone provided greater protection than NR alone. The protection afforded by the combination of 1.1 μM compound I-86+NR was stronger than, and statistically different from, the protection observed with NR alone. The protection afforded by the combination of 3.3 μM compound I-86+NR was stronger than, and statistically different from, the protection observed with 3.3 μM compound I-86 alone. In FIG. 2B, at 24 h, 1.1 μM compound I-86 alone provided less protection than NR alone, whereas 3.3 μM compound I-86 alone and 10 μM compound I-86 offered similar protection to NR alone. The protection afforded by the combinations of compound I-86+NR (3.3 μM compound I-86+NR and 10 μM compound I-86+NR) was stronger than, and statistically significant from the protection observed with either compound I-86 or NR alone.

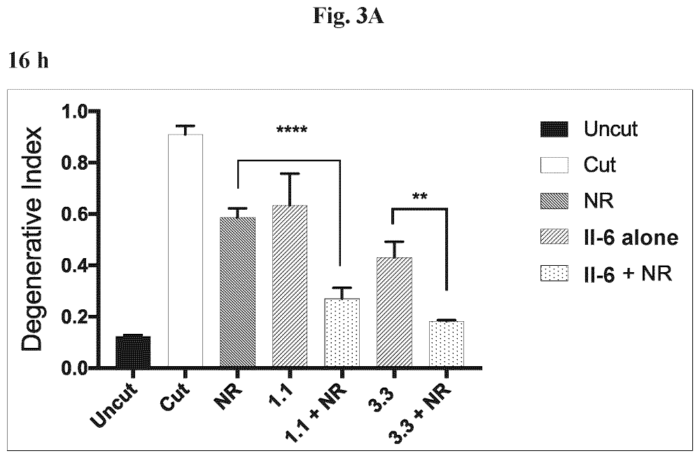

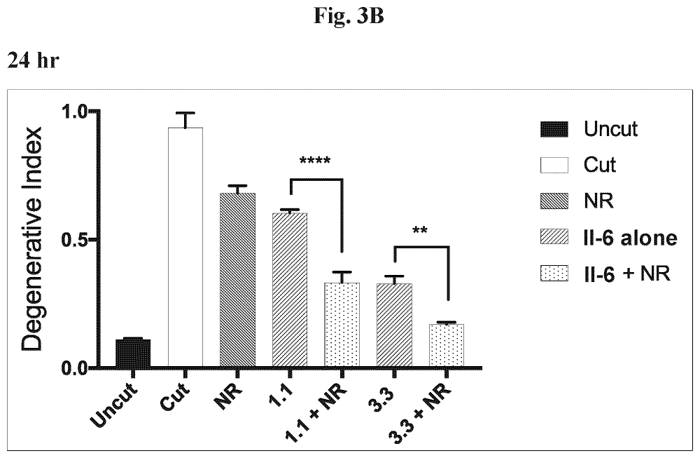

The efficacy SARM1 inhibitors when applied in combination with NR on the axon degeneration assay described herein was tested on two additional isothiazole compounds. The SARM1 inhibitor II-6 was tested on the axon degeneration assay in combination with 100 μM NR. The effect of a potent SARM1 inhibitor FIGS. 3A and 3B show the degeneration index of DRG axons at 16 and 24 hours post-axotomy, respectively. For each concentration of compound II-6 tested, the extent of axonal protection of a combination of compound II-6+NR was always compared to the amount of protection produced by the agent in that combination that, individually, had the greater protective effect. In FIG. 3A, at 16 h the protection afforded by the combination of 1.1 μM compound II-6+NR was stronger than, and statistically different from, the protection observed with either compound II-6 or NR alone. At 3.3 μM, compound II-6 alone showed stronger protection that NR alone, however, the protection afforded by the combination of 3.3 μM compound II-6+NR was stronger than, and statistically different from, the protection observed with 3.3 μM compound II-6 alone. In FIG. 3B, at 24 h, the protection afforded by the combination of 1.1 μM compound II-6+NR was stronger than, and statistically different from, the protection observed with either compound II-6 or NR alone. At 3.3 μM, compound II-6 alone showed stronger protection than NR alone, however, the protection afforded by the combination of 3.3 μM compound II-6+NR was stronger than, and statistically different from, the protection observed with 3.3 μM compound II-6 alone. Similar to the Isoquinoline SARM1 inhibitors, a given concentration of II-6 provided better axonal protection when combined with 100 μM NR, than either treatment alone.

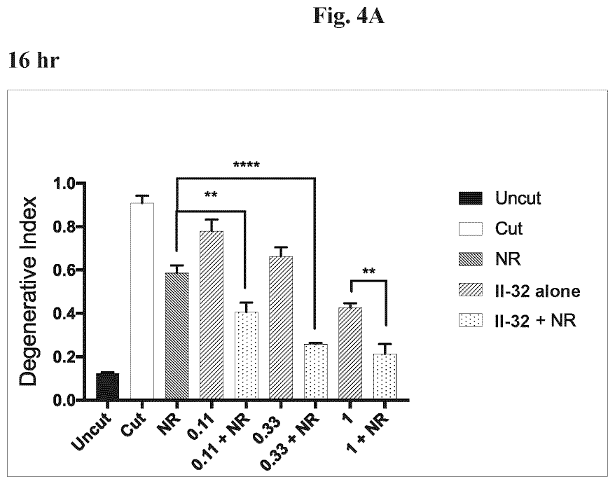

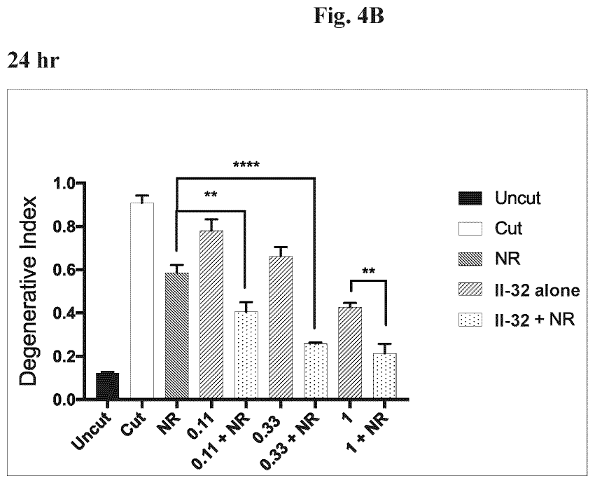

The effect of combining a SARM1 inhibitor with NR was further tested with the SARM1 inhibitor II-32 in combination with 100 μM NR on the axon degeneration assay described herein. The combination of compound II-32+NR extends neuroprotection post-axotomy as compared to single agent therapy. FIGS. 4A and 4B show the degeneration index of DRG axons at 16 and 24 hours post-axotomy, respectively. For each concentration of compound II-32 tested, the extent of axonal protection of a combination of compound II-32+NR was always compared to the amount of protection produced by the agent in that combination that, individually, had the greater protective effect. In FIG. 4A, at 16 h, NR alone provided greater protection than 0.11 μM and 0.33 compound II-32 alone, whereas 1 μM compound II-32 alone provided greater protection than NR alone. The protection afforded by each combination of 0.11 μM compound II-32+NR and 0.33 μM compound II-32+NR was stronger than, and statistically different from, the protection observed with NR alone. Similarly, the protection afforded by the combination of 1 μM compound II-32+NR was stronger than, and statistically different from, the protection observed with 1 μM compound II-32 alone. In FIG. 4B, at 24 h, NR alone provided greater protection than 0.11 μM and 0.33 μM compound II-32 alone, whereas 1 μM compound II-32 alone provided greater protection than NR alone. The protection afforded by the combinations of 0.11 μM compound II-32+NR and 0.33 μM compound II-32+NR was stronger than, and statistically different from, the protection observed with NR alone. Similarly, the protection afforded by the combination of 1 μM compound II-32+NR was statistically better than the protection observed with 1 μM compound II-32 alone.

Taken together, these results demonstrate the neuroprotective efficacy of SARM1 inhibitors when provided in combination with NR on the axon degeneration assay described herein.

1. (canceled)

2. (canceled)

3. (canceled)

4. A method for treating and/or preventing axonal degeneration comprising administering to a patient in need thereof a SARM1 inhibitor in combination with NAD+ or a NAD+ precursor.

5. A method comprising administering to a patient at risk for developing a neurodegenerative disease or disorder a SARM1 inhibitor in combination with NAD+ or a NAD+ precursor.

6. The method according to claim 5, wherein the NAD+ precursor is NR, NA, NaR, NAM, NMN, NaMN, TRP, vitamin B₃, or NAAD.

7. The method according to claim 6, wherein the NAD+ precursor is NR.

8. The method according to claim 5, wherein the SARM1 inhibitor is selected from a small molecule, a nucleic acid, a polypeptide, a peptide fragment, an antibody or a ribozyme.

9. The method according to claim 8, wherein the nucleic acid is selected from a siRNA, an antisense oligonucleotide, a micro-RNA, or an aptamer.

10. The method according to claim 5, wherein the neurodegenerative disease or disorder is selected from an acute or chronic peripheral nervous system disease or disorder, an acute or chronic central nervous system disease or disorder, or a disease associated with neurodegeneration.

11. The method according to claim 5, wherein the neurodegenerative disease is a chronic disease or disorder of the peripheral nervous system selected from a systemic disorder, a pain disorder, or a metabolic disease or disorder,
wherein the systemic disorder is selected from diabetes, uremia, infectious diseases such as AIDS or leprosy, nutritional deficiencies, vascular or collagen disorders such as atherosclerosis, enteric neuropathies and axonopathies, Guillain-Barre syndrome, severe acute motor axonal neuropathy (AMAN), and autoimmune diseases such as systemic lupus erythematosus, scleroderma, sarcoidosis, rheumatoid arthritis, and polyarteritis nodosa
wherein the pain disorder is selected from chronic pain, fibromyalgia, spinal pain, carpal tunnel syndrome, pain from cancer, arthritis, sciatica, headaches, pain from surgery, muscle spasms, back pain, visceral pain, pain from injury, dental pain, neuralgia, such as neurogenic or neuropathic pain, nerve inflammation or damage, shingles, herniated disc, torn ligament, and diabetes.
wherein the metabolic disease or disorder is selected from diabetes mellitus, hypoglycemia, uremia, hypothyroidism, hepatic failure, polycythemia, amyloidosis, acromegaly, porphyria, disorders of lipid/glycolipid metabolism, nutritional/vitamin deficiencies, and mitochondrial disorders.

12. The method according to claim 5, wherein the neurodegenerative disease is an acute disease or disorder of the peripheral nervous system selected from mechanical injuries, thermal injury, and chemical injury or chemotherapy induced neuropathy (CIPN),
wherein mechanical injuries are selected from compression or entrapment injuries such as carpal tunnel syndrome, direct trauma, penetrating injuries, contusions, fractures or dislocated bones; pressure involving superficial nerves or from a tumor; or a traumatic neuronal injury resulting from increased intraocular pressure.
wherein agents that induce chemical injury or chemotherapy induced neuropathy (CIPN) are selected from cytotoxic anticancer agents, thalidomide, epothilones (e.g., ixabepilone), taxanes (e.g., paclitaxel and docetaxel), vinca alkaloids (e.g., vinblastine, vinorelbine, vincristine, and vindesine), proteasome inhibitors (e.g., bortezomib), platinum-based drugs (e.g., cisplatin, oxaliplatin, and carboplatin) and auristatins (e.g. conjugated monomethyl auristatin E).

13. The method according to claim 5, wherein the neurodegenerative disease is a chronic disease or disorder of the central nervous system, including a central nervous system disorder, an optic nerve disorder, a traumatic brain injury, or metabolic disease or disorder.
wherein a chronic central nervous system disorder is selected from Alzheimer's disease, Parkinson's disease, amyotrophic lateral sclerosis (ALS, Lou Gehrig's disease), multiple sclerosis, Huntington's disease, senile dementia, Pick's disease, Gaucher's disease, Hurler Syndrome, progressive multifocal leukoencephalopathy, Alexander's disease, congenital hypomyelination, encephalomyelitis, acute disseminated encephalomyelitis, central pontine myelolysis, osmotic hyponatremia, Tay-Sachs disease, motor neuron disease, ataxia, spinal muscular atrophy (SMA), Niemann-Pick disease, acute hemorrhagic leukoencephalitis, trigeminal neuralgia, Bell's palsy, cerebral ischemia, multiple system atrophy, Pelizaeus Merzbacher disease, periventricular leukomalacia, hereditary ataxias, noise induced hearing loss, Creutzfeldt-Jakob disease, transmissible spongiform encephalopathy, congenital hearing loss, age-related hearing loss, Lewy Body Dementia, frontotemporal dementia, amyloidosis, diabetic neuropathy, globoid cell leukodystrophy (Krabbe's disease), Bassen-Kornzweig syndrome, transverse myelitis, Charcot-Marie-Tooth disease, motor neuron disease, spinocerebellar ataxias, pre-eclampsia, hereditary spastic paraplegias, spastic paraparesis, familial spastic paraplegia, French settlement disease, Strumpell-Lorrain disease, non-alcoholic steatohepatitis (NASH), hereditary sensory and autonomic neuropathy (HSAN), adrenomyeloneuropathy, progressive supra nuclear palsy (PSP), Friedrich's ataxia, or caused by a somatic mutation or idiopathic condition.
wherein the optic nerve disorder is selected from an acute optic neuropathy (AON), a genetic or idiopathic retinal condition, Leber's congenital amaurosis, Leber's hereditary optic neuropathy, primary open angle glaucoma, acute angle closure glaucoma, autosomal dominant optic atrophy, retinal ganglion degeneration, retinitis pigmentosa and outer retinal neuropathies, optic nerve neuritis and/or degeneration including that associated with multiple sclerosis, Kjer's disease, ischemic optic neuropathies, deficiencies in vitamins B12 or folic acid, isolated vitamin E deficiency syndrome, non-arteritic anterior ischemic optic neuropathy, and exposure to ethambutol or cyanide.
wherein the traumatic brain injury is selected from chronic injury to the central nervous system, spinal cord injury, traumatic axonal injury and chronic traumatic encephalopathy (CTE).
wherein the metabolic disease or disorder is selected from diabetes mellitus, hypoglycemia, Bassen-Kornzweig syndrome, uremia, hypothyroidism, hepatic failure, polycythemia, amyloidosis, acromegaly, porphyria, disorders of lipid/glycolipid metabolism, nutritional/vitamin deficiencies, and mitochondrial disorders.

14. The method according to claim 5, wherein the neurodegenerative disease is an acute disease or disorder of the central nervous system selected from ischemia or stroke, traumatic brain injury, chemical injury, thermal injury, and viral encephalitides.
wherein ischemia or stroke includes acute ischemia, cerebral ischemia, hypoxic demyelination, ischemic demyelination, ischemic optic neuropathies, non-arteritic anterior ischemic optic neuropathy
wherein the traumatic brain injuries are selected from injuries to the spinal cord and/or traumatic brain injury, mechanical injuries or traumatic injuries to the head and spine, blunt force trauma, closed-head injury, open head injury, exposure to a concussive and/or explosive force, a penetrating injury in or to the brain cavity or innervated region of the body, a force which causes axons to deform, stretch, crush or sheer, or increased intraocular pressure.
wherein viral encephalitidies include enteroviruses, arboviruses, herpes simplex virus, West Nile virus encephalitis, La Crosse virus encephalitis, Bunyavirus encephalitis, pediatric viral encephalitis, and AIDS dementia complex (also known as HIV dementia, HIV encephalopathy, and HIV-associated dementia).

15. The method according to claim 5, wherein the neurodegenerative disease or disorder results from blood clotting, inflammation, flushing, obesity, aging, stress, cancer, diabetes, pain.

16. The method according to claim 5, wherein the patient is a human.

17. The method of claim 16, wherein the patient is a subject or population at risk of developing a condition involving axonal degeneration.

18. The method of claim 17, wherein the risk of developing a condition involving axonal degeneration is selected from age, one or more genetic risk factors for neurodegeneration, family history, engaging in one or more high-risk activities, or one or more biomarkers for neurodegeneration.

19. The method of claim 18, wherein the one or more genetic risk factors for neurodegeneration is selected from one or more copies of a known genetic risk factor, a hexanucleotide repeat expansion in chromosome 9 open reading frame 72 or one or more copies of the ApoE4 allele.

20. The method of claim 18, wherein the one or more high risk activities is selected from American football, basketball, boxing, diving, field hockey, football, ice hockey, lacrosse, martial arts, rodeo, rugby, ski jumping, water polo, wrestling, baseball, cycling, cheerleading, fencing, track and field, gymnastics, handball, horseback riding, skating, skiing, skateboarding, softball, squash, ultimate frisbee, volleyball, and/or windsurfing.

21. The method of claim 18, wherein the one or more biomarkers of neurodegeneration is selected from the concentration of neurofilament light chain protein (NF-L) and/or neurofilament heavy chain protein (NF-H) contained in the cerebral spinal fluid, blood, and/or plasma of a subject; constitutive NAD+ and/or cADPR levels in neurons and/or axons; levels of albumin, amyloid-β (Aβ)38, Aβ40, Aβ42, glial fibrillary acid protein (GFAP), heart-type fatty acid binding protein (hFABP), monocyte chemoattractin protein (MCP)-1, neurogranin, neuron specific enolayse (NSE), soluble amyloid precursor protein (sAPP)α, sAPPP, soluble triggering receptor expressed on myeloid cells (sTREM) 2, phospho-tau, and/or total-tau; cytokines and/or chemokines, including Ccl2, Ccl7, Ccl12, Csf1, and/or 116.

22. A kit comprising a first container, a second container and a package insert, wherein the first container comprises at least one dose of a medicament comprising a SARM1 inhibitor, the second container comprises at least one dose of a medicament comprising NAD+ or a NAD+ precursor, and the package insert comprises instructions for treating neurodegeneration using the medicaments.

23. The kit according to claim 22, wherein the instructions state that the medicaments are intended for use in treating a patient at risk of axonal degeneration.

24. The kit according to claim 22, wherein the NAD+ precursor is NR, NA, NaR, NAM, NMN, NaMN, TRP, vitamin B₃, or NAAD.

25. The kit according to claim 24, wherein the NAD+ precursor is NR.

26. The method according to claim 4, wherein the NAD+ precursor is NR, NA, NaR, NAM, NMN, NaMN, TRP, vitamin B₃, or NAAD.

27. The method according to claim 26, wherein the NAD+ precursor is NR.

28. The method according to claim 27, wherein the SARM1 inhibitor is a nucleic acid selected from a siRNA, an antisense oligonucleotide, a micro-RNA, or an aptamer.
